SEQUENCE LISTING (177 sequences)

SEQ ID NO: 1 - protein, 53 aa, artificial
  Antimicrobial peptides Ac3 and Ac5 expressed in phage clone Ac2r5c5
FVSFHTTNHVIGFNRLDFLKCISGTICFKCPYFHFPKTLSTKLCFTTKRLLRY

SEQ ID NO: 2 - protein, 23 aa, artificial
  Antimicrobial peptide Ac3
FVSFHTTNHVIGFNRLDFLKCIS

SEQ ID NO: 3 - protein, 23 aa, artificial
  Antimicrobial peptide Ac5
PYFHFPKTLSTKLCFTTKRLLRY

SEQ ID NO: 4 - protein, 55 aa, artificial
  Antimicrobial peptides Ac8 and Ac9 expressed in phage clone Ac2r5c9
QPPSSLKEPGRMNTHKHARLTFLRRLEMVQQLPGRMNTHKHARLTFLRRLEMVQQ

SEQ ID NO: 5 - protein, 18 aa, artificial
  Consensus sequence for peptides Ac8 and Ac9
THKHARLTFLRRLEMVQQ

SEQ ID NO: 6 - protein, 19 aa, artificial
  Antimicrobial peptide Ac8
NTHKHARLTFLRRLEMVQQ

SEQ ID NO: 7 - protein, 23 aa, artificial
  Antimicrobial peptide Ac9
THKHARLTFLRRLEMVQQLPGRM

SEQ ID NO: 8 - protein, 48 aa, artificial
  Antimicrobial peptides Ac13 and Ac15 expressed in phage clone Ac2r5c34
CSFCEKTGKNGCVRYNHSFFAVCRQLLSKIRALRRRRFGQCDKRISYK

SEQ ID NO: 9 - protein, 10 aa, artificial
  Consensus sequence for peptides Ac13, Ac14, Ac15 and Ac16
LLSKIRALRR

SEQ ID NO: 10 - protein, 23 aa, artificial
  Antimicrobial peptide Ac13
VRYNHSFFAVCRQLLSKIRALRR

SEQ ID NO: 11 - protein, 23 aa, artificial
  Antimicrobial peptide Ac14 (equivalent to Ac13-C11S)
VRYNHSFFAVSRQLLSKIRALRR

SEQ ID NO: 12 - protein, 23 aa, artificial
  Antimicrobial peptide Ac15
LLSKIRALRRRRFGQCDKRISYK

SEQ ID NO: 13 - protein, 23 aa, artificial
  Antimicrobial peptide Ac16 (equivalent to Ac15-C16S)
LLSKIRALRRRRFGQSDKRISYK

SEQ ID NO: 14 - protein, 55 aa, artificial
  Antimicrobial peptides Ac17 and Ac18 expressed in phage clone Ac2r5c38
VRECRRVGGKQSGIYKYIIRLIENWGECGCRYCRIVRPLVRIAADRRVVERSEQQ

SEQ ID NO: 15 - protein, 9 aa, artificial
  Consensus sequence for peptides Ac17, Ac18, Ac19
YKYIIRLIE

SEQ ID NO: 16 - protein, 23 aa, artificial
  Antimicrobial peptide Ac17
VRECRRVGGKQSGIYKYIIRLIE

SEQ ID NO: 17 - protein, 23 aa, artificial
  Antimicrobial peptide Ac18
YKYIIRLIENWGECGCRYCRIVR

SEQ ID NO: 18 - protein, 23 aa, artificial
  Antimicrobial peptide Ac19 (equivalent to peptide Ac18-C14S/C16S/C19S)
YKYIIRLIENWGESGSRYSRIVR

SEQ ID NO: 19 - protein, 62 aa, artificial
  Antimicrobial peptide Ac35 expressed in phage clone Ac3r5c8
PDNAGNHQQYKKYQGKVPTHRVGRLSGSICLRLAGWRGTPLAGFPRCRPASCASCRPSALPA

SEQ ID NO: 20 - protein, 23 aa, artificial
  Antimicrobial peptide Ac35
KKYQGKVPTHRVGRLSGSICLRL

SEQ ID NO: 21 - protein, 27 aa, artificial
  Antimicrobial peptides Ac38 and Ac40 expressed in phage clone Ac3r5c10
VRGARLRCALLCSDWIVSCRLLGVNCQ

SEQ ID NO: 22 - protein, 19 aa, artificial
  Consensus sequence for peptides Ac38 and Ac40
RLRCALLCSDWIVSCRLLG

SEQ ID NO: 23 - protein, 23 aa, artificial
  Antimicrobial peptide Ac38
VRGARLRCALLCSDWIVSCRLLG

SEQ ID NO: 24 - protein, 23 aa, artificial
  Antimicrobial peptide fragment Ac39 (equivalent to Ac38-C8S/C12S/C19S)
VRGARLRSALLSSDWIVSSRLLG

SEQ ID NO: 25 - protein, 23 aa, artificial
  Antimicrobial peptide Ac40
GARLRCALLCSDWIVSCRLLGVN

SEQ ID NO: 26 - protein, 23 aa, artificial
  Antimicrobial peptide Ac41 (modified form of SEQ ID NO 21 with cysteines modified to serines)
RLRSALLSSDWIVSSRLLGVNSQ

SEQ ID NO: 27 - protein, 29 aa, artificial
  Antimicrobial peptide Ac44 expressed in phage clone designated Ac4r5c1
HPCTTPNTPPTTSPTPPPPPPSAPASGQR

SEQ ID NO: 28 - protein, 23 aa, artificial
  Antimicrobial peptide Ac44
HPCTTPNTPPTTSPTPPPPPPSA

SEQ ID NO: 29 - protein, 29 aa, artificial
  Antimicrobial peptides Ac46 and Ac47 expressed in phage clone Ac4r5c10
HSCTTPNTPQQPHPHHRHRRHRHRHPGTA

SEQ ID NO: 30 - protein, 17 aa, artificial
  Consensus sequence for Ac46 and Ac47
NTPQQPHPHHRHRRHRH

SEQ ID NO: 31 - protein, 23 aa, artificial
  Antimicrobial peptide Ac46
HSCTTPNTPQQPHPHHRHRRHRH

SEQ ID NO: 32 - protein, 23 aa, artificial
  Antimicrobial peptide Ac47
NTPQQPHPHHRHRRHRHRHPGTA

SEQ ID NO: 33 - protein, 47 aa, artificial
  Antimicrobial peptide Ac53 expressed in phage clone Ac4r5c59
LAMPVRGQTPLPRKLHEGTRRRHHRHTPGKRHRALTRTQRLTGQMQS

SEQ ID NO: 34 - protein, 23 aa, artificial
  Antimicrobial peptide Ac53
QTPLPRKLHEGTRRRHHRHTPGK

SEQ ID NO: 35 - protein, 54 aa, artificial
  Antimicrobial peptide Ac56 expressed in phage clone Ac4r5c68
TAGIGTPTTWHHHYTQTHPHPHNRRSRMSRARILSAPTVDIQQTAPRDNKLSNC

SEQ ID NO: 36 - protein, 23 aa, artificial
  Antimicrobial peptide Ac56
WHHHYTQTHPHPHNRRSRMSRAR

SEQ ID NO: 37 - protein, 39 aa, artificial
  Antimicrobial peptide Ac59 expressed in phage clone Ac4r5c71
LGKRSEAGIKLASPLAWRVAMPTRFLHTCRRFFVPKKVH

SEQ ID NO: 38 - protein, 23 aa, artificial
  Antimicrobial peptide Ac59
WRVAMPTRFLHTCRRFFVPKKVH

SEQ ID NO: 39 - protein, 40 aa, artificial
  Antimicrobial peptides Ac66, Ac67 and Ac68 expressed in phage clone Ac5r4c2
WGRTPSPRSPPGRSSGRGRSASCALRWAGPLPKGRGKWWC

SEQ ID NO: 40 - protein, 6 aa, artificial
  Consensus sequence for Ac66, Ac67, Ac68
GRSASC

SEQ ID NO: 41 - protein, 17 aa, artificial
  Consensus sequence for Ac66 and Ac67
PRSPPGRSSGRGRSASC

SEQ ID NO: 42 - protein, 12 aa, artificial
  Consenses sequence for Ac67 and Ac68
GRSASCALRWAG

SEQ ID NO: 43 - protein, 23 aa, artificial
  Antimicrobial peptide Ac66
WGRTPSPRSPPGRSSGRGRSASC

SEQ ID NO: 44 - protein, 23 aa, artificial
  Antimicrobial peptide Ac67
PRSPPGRSSGRGRSASCALRWAG

SEQ ID NO: 45 - protein, 23 aa, artificial
  Antimicrobial peptide Ac68
GRSASCALRWAGPLPKGRGKWWC

SEQ ID NO: 46 - protein, 19 aa, artificial
  Antimicrobial peptide Ac88 expressed in phage clone Ac5r4c23
FFFLLKMNYNATHLAGREF

SEQ ID NO: 47 - protein, 19 aa, artificial
  Antimicrobial peptide Ac88
FFFLLKMNYNATHLAGREF

SEQ ID NO: 48 - protein, 14 aa, artificial
  Antimicrobial peptide Ac96 expressed in phage clone Ac5r4c39
GDPNCFSRVKILAC

SEQ ID NO: 49 - protein, 14 aa, artificial
  Antimicrobial peptide Ac96 expressed in phage clone Ac5r4c39
GDPNCFSRVKILAC

SEQ ID NO: 50 - protein, 14 aa, artificial
  Antimicrobial peptide fragment Ac96
GDPNCFSRVKILAC

SEQ ID NO: 51 - protein, 53 aa, artificial
  Antimicrobial peptides Ac97, Ac98, Ac99 and Ac100 expressed in phage clone Ac5r4c54
HWMIPLRKGAQYEELRKLSKGDHLVKLKTSPQARKKWPGLGNEVTARLLTVSA

SEQ ID NO: 52 - protein, 15 aa, artificial
  Consensus sequence for Ac97 and Ac98
GAQYEELRKLSKGDH

SEQ ID NO: 53 - protein, 11 aa, artificial
  Consensus sequence for Ac97 and Ac99
GDHLVKLKTSP

SEQ ID NO: 54 - protein, 13 aa, artificial
  Consensus sequence for Ac99 and Ac100
PQARKKWPGLGNE

SEQ ID NO: 55 - protein, 23 aa, artificial
  Antimicrobial peptide Ac97
HWMIPLRKGAQYEELRKLSKGDH

SEQ ID NO: 56 - protein, 23 aa, artificial
  Antimicrobial peptide  Ac98
GAQYEELRKLSKGDHLVKLKTSP

SEQ ID NO: 57 - protein, 23 aa, artificial
  Antimicrobial peptide Ac99
GDHLVKLKTSPQARKKWPGLGNE

SEQ ID NO: 58 - protein, 23 aa, artificial
  Antimicrobial peptide  Ac100
PQARKKWPGLGNEVTARLLTVSA

SEQ ID NO: 59 - protein, 22 aa, artificial
  Antimicrobial peptide Ac101 expressed in phage clone Ac5r4c57
ANLRSKKQKGKKWNHWPRSIKA

SEQ ID NO: 60 - protein, 22 aa, artificial
  Antimicrobial peptide  Ac101
ANLRSKKQKGKKWNHWPRSIKA

SEQ ID NO: 61 - protein, 32 aa, artificial
  Antimicrobial peptides Ac102 and Ac103 expressed in phage clone Ac5r4c60
FVCVKDTISGCRRITGFCVGWRLLLLLTGSGV

SEQ ID NO: 62 - protein, 14 aa, artificial
  Consensus sequence for Ac102 and Ac103
GCRRITGFCVGWRL

SEQ ID NO: 63 - protein, 23 aa, artificial
  Antimicrobial peptide Ac102
FVCVKDTISGCRRITGFCVGWRL

SEQ ID NO: 64 - protein, 23 aa, artificial
  Antimicrobial peptide Ac103
GCRRITGFCVGWRLLLLLTGSGV

SEQ ID NO: 65 - protein, 33 aa, artificial
  Antimicrobial peptide Ac116 expressed in phage clone Ac5r4c71
FKLIPTRRSSLVKSLPAFRRKAPQSVSGSTNRA

SEQ ID NO: 66 - protein, 23 aa, artificial
  Antimicrobial peptide Ac116
FKLIPTRRSSLVKSLPAFRRKAP

SEQ ID NO: 67 - protein, 63 aa, artificial
  Antimicrobial peptide Ac138 expressed in phage clone Ac5r5c39
GLSGHEPRRLSAQLAADLFRHRLGAGSAGGARLEMGGAVRPDHHAGCGLCLARANTTCIPSRA

SEQ ID NO: 68 - protein, 23 aa, artificial
  Antimicrobial peptide  Ac138
GLSGHEPRRLSAQLAADLFRHRL

SEQ ID NO: 69 - protein, 26 aa, artificial
  Antimicrobial peptide Ac173 expressed in phage clone Ac5r5c79
TLQRRQVRDLGLRVHRHFSQFINRSG

SEQ ID NO: 70 - protein, 23 aa, artificial
  Antimicrobial peptide  Ac173
RRQVRDLGLRVHRHFSQFINRSG

SEQ ID NO: 71 - protein, 22 aa, artificial
  Antimicrobial peptide Ac193
PHTSRYFPSSRYSFFRSKASMA

SEQ ID NO: 72 - protein, 23 aa, artificial
  Antimicrobial peptide Ac195
IPVWFMRQAGRYLNVYKDYRKNT

SEQ ID NO: 73 - protein, 23 aa, artificial
  Antimicrobial peptide Ac228
CCNAIIYKWILHFFWRKRAWYCF

SEQ ID NO: 74 - protein, 23 aa, artificial
  Antimicrobial peptide Ac228-C1S/C2S/C22S
SSNAIIYKWILHFFWRKRAWYSF

SEQ ID NO: 75 - protein, 23 aa, artificial
  Antimicrobial peptide Ac259
RSNLPIHNILRYLFYGKVKNKRL

SEQ ID NO: 76 - protein, 23 aa, artificial
  Antimicrobial peptide Ac319
LPPDIMYSEASSHSLSVLPIPFF

SEQ ID NO: 77 - protein, 47 aa, artificial
  Antimicrobial peptides Ac319, Ac322 and Ac323 in phage Ac6b5/S
LPPDIMYSEASSHSLSVLPIPFFNRTGFFCLPKNLKRSKFCAFLDPT

SEQ ID NO: 78 - protein, 5 aa, artificial
  Consensus sequence for Ac319 and Ac322
PIPFF

SEQ ID NO: 79 - protein, 17 aa, artificial
  Consensus sequence for Ac322 and Ac323
RTGFFCLPKNLKRSKFC

SEQ ID NO: 80 - protein, 23 aa, artificial
  Antimicrobial peptide Ac322
PIPFFNRTGFFCLPKNLKRSKFC

SEQ ID NO: 81 - protein, 23 aa, artificial
  Antimicrobial peptide Ac322-C12S/C23S
PIPFFNRTGFFSLPKNLKRSKFS

SEQ ID NO: 82 - protein, 23 aa, artificial
  Antimicrobial peptide Ac323
RTGFFCLPKNLKRSKFCAFLDPT

SEQ ID NO: 83 - protein, 23 aa, artificial
  Antimicrobial peptide Ac323-C6S/C17S
RTGFFSLPKNLKRSKFSAFLDPT

SEQ ID NO: 84 - protein, 37 aa, artificial
  Antimicrobial peptides Ac327, Ac328 and Ac329 in phage Ac6b10/S
PHSSRAASSRCAVRPVWPSGFLRRLSDRCCIHSRQAQ

SEQ ID NO: 85 - protein, 9 aa, artificial
  Consensus sequence for Ac327, Ac328 and Ac329
PVWPSGFLR

SEQ ID NO: 86 - protein, 16 aa, artificial
  Consensus sequence for Ac327 and Ac328
SSRCAVRPVWPSGFLR

SEQ ID NO: 87 - protein, 16 aa, artificial
  Consensus sequence for Ac328 and Ac329
PVWPSGFLRRLSDRCC

SEQ ID NO: 88 - protein, 23 aa, artificial
  Antimicrobial peptide Ac327
PHSSRAASSRCAVRPVWPSGFLR

SEQ ID NO: 89 - protein, 23 aa, artificial
  Antimicrobial peptide Ac328
SSRCAVRPVWPSGFLRRLSDRCC

SEQ ID NO: 90 - protein, 23 aa, artificial
  Antimicrobial peptide Ac328-C4S/C22S/C23S
SSRSAVRPVWPSGFLRRLSDRSS

SEQ ID NO: 91 - protein, 23 aa, artificial
  Antimicrobial peptide Ac329
PVWPSGFLRRLSDRCCIHSRQAQ

SEQ ID NO: 92 - protein, 23 aa, artificial
  Antimicrobial peptide Ac329-C15S/C16S
PVWPSGFLRRLSDRSSIHSRQAQ

SEQ ID NO: 93 - protein, 23 aa, artificial
  Antimicrobial peptide Ac330
GGPHCGYFATTKKLMRKVPGRLV

SEQ ID NO: 94 - protein, 33 aa, artificial
  Antimicrobial peptides Ac338, Ac339 and Ac340 in phage Ac6b14/S
GGVVFGVLTFSGRLREDLRRICRQISAYKSDSS

SEQ ID NO: 95 - protein, 13 aa, artificial
  Consensus sequence for Ac338, Ac339 and Ac340
SGRLREDLRRICR

SEQ ID NO: 96 - protein, 18 aa, artificial
  Consensus sequence for Ac338 and Ac339
GVLTFSGRLREDLRRICR

SEQ ID NO: 97 - protein, 18 aa, artificial
  Consensus sequence for Ac339 and Ac340
SGRLREDLRRICRQISAY

SEQ ID NO: 98 - protein, 23 aa, artificial
  Antimicrobial peptide Ac338
GGVVFGVLTFSGRLREDLRRICR

SEQ ID NO: 99 - protein, 23 aa, artificial
  Antimicrobial peptide Ac339
GVLTFSGRLREDLRRICRQISAY

SEQ ID NO: 100 - protein, 23 aa, artificial
  Antimicrobial peptide Ac340
SGRLREDLRRICRQISAYKSDSS

SEQ ID NO: 101 - protein, 46 aa, artificial
  Antimicrobial peptides Ac364 and Ac370 in phage Ac6b33/S
TRKRCAARPTTCRCWSIGRAWKAMGRLSGVAWRRLSLRHCECGRRP

SEQ ID NO: 102 - protein, 46 aa, artificial
  Antimicrobial peptides Ac364 and Ac370 in phage Ac6b33/S
MGRLSGVAWRRLSLRHCECGRRPTRKRCAARPTTCRCWSIGRAWKA

SEQ ID NO: 103 - protein, 23 aa, artificial
  Antimicrobial peptide Ac364
TRKRCAARPTTCRCWSIGRAWKA

SEQ ID NO: 104 - protein, 23 aa, artificial
  Antimicrobial peptide Ac364-C5S/C12S/C14S
TRKRSAARPTTSRSWSIGRAWKA

SEQ ID NO: 105 - protein, 23 aa, artificial
  Antimicrobial peptide Ac370
MGRLSGVAWRRLSLRHCECGRRP

SEQ ID NO: 106 - protein, 23 aa, artificial
  Antimicrobial peptide Ac370-C17S/C19S
MGRLSGVAWRRLSLRHSESGRRP

SEQ ID NO: 107 - protein, 34 aa, artificial
  Antimicrobial peptides Ac378, Ac379 and Ac380 in phage Ac6b11/S
SDLGKSGAGWRWRACNTLSKCRGSCGLRVWAGRG

SEQ ID NO: 108 - protein, 12 aa, artificial
  Consensus sequence for Ac378, Ac379 and Ac380
WRACNTLSKCRG

SEQ ID NO: 109 - protein, 17 aa, artificial
  Consensus sequence for Ac378 and Ac379
GAGWRWRACNTLSKCRG

SEQ ID NO: 110 - protein, 18 aa, artificial
  Consensus sequence for Ac379 and Ac380
WRACNTLSKCRGSCGLRV

SEQ ID NO: 111 - protein, 23 aa, artificial
  Antimicrobial peptide Ac378
SDLGKSGAGWRWRACNTLSKCRG

SEQ ID NO: 112 - protein, 23 aa, artificial
  Antimicrobial peptide Ac378-C15S/C21S
SDLGKSGAGWRWRASNTLSKSRG

SEQ ID NO: 113 - protein, 23 aa, artificial
  Antimicrobial peptide Ac379
GAGWRWRACNTLSKCRGSCGLRV

SEQ ID NO: 114 - protein, 23 aa, artificial
  Antimicrobial peptide Ac379-C9S/C15S/C19S
GAGWRWRASNTLSKSRGSSGLRV

SEQ ID NO: 115 - protein, 23 aa, artificial
  Antimicrobial peptide Ac380
WRACNTLSKCRGSCGLRVWAGRG

SEQ ID NO: 116 - protein, 23 aa, artificial
  Antimicrobial peptide Ac380-C4S/C10S/C14S
WRASNTLSKSRGSSGLRVWAGRG

SEQ ID NO: 117 - protein, 30 aa, artificial
  Antimicrobial peptides Ac389 and Ac390 in phage Ac6b29/L
GYRRSRRSRWICSMARRSRRRRPGRSTRSW

SEQ ID NO: 118 - protein, 16 aa, artificial
  Consensus sequence for Ac389 and Ac390
SRWICSMARRSRRRRP

SEQ ID NO: 119 - protein, 23 aa, artificial
  Antimicrobial peptide Ac389
GYRRSRRSRWICSMARRSRRRRP

SEQ ID NO: 120 - protein, 23 aa, artificial
  Antimicrobial peptide Ac390
SRWICSMARRSRRRRPGRSTRSW

SEQ ID NO: 121 - protein, 19 aa, artificial
  Antimicrobial peptide Ac431
CHQPTMRCCLSHIRRRFRL

SEQ ID NO: 122 - protein, 19 aa, artificial
  Antimicrobial peptide Ac431-C1S/C8S/C9S
SHQPTMRSSLSHIRRRFRL

SEQ ID NO: 123 - protein, 23 aa, artificial
  Antimicrobial peptide Ac434
VLPVAMLRATLRATVPISRSRLR

SEQ ID NO: 124 - protein, 30 aa, artificial
  Antimicrobial peptides Ac436 and Ac437 in phage Ac6c28/L
WGVNRMPPVVRQILRSEWRNWRKSLEMRGR

SEQ ID NO: 125 - protein, 16 aa, artificial
  Consensus sequence for Ac436 and Ac437
PVVRQILRSEWRNWRK

SEQ ID NO: 126 - protein, 23 aa, artificial
  Antimicrobial peptide Ac436
WGVNRMPPVVRQILRSEWRNWRK

SEQ ID NO: 127 - protein, 23 aa, artificial
  Antimicrobial peptide Ac437
PVVRQILRSEWRNWRKSLEMRGR

SEQ ID NO: 128 - protein, 23 aa, artificial
  Antimicrobial peptide Ac451
GWFPAAGCGWACCRLRMAGPRIG

SEQ ID NO: 129 - protein, 23 aa, artificial
  Antimicrobial peptide Ac451-C8S/C12S/C13S
GWFPAAGSGWASSRLRMAGPRIG

SEQ ID NO: 130 - protein, 23 aa, artificial
  Antimicrobial peptide Ac469
RPRPVDHRAGSLRWAIAFQVSSL

SEQ ID NO: 131 - protein, 13 aa, artificial
  Antimicrobial peptide Ac472
HNTCFLTWFTTCL

SEQ ID NO: 132 - protein, 13 aa, artificial
  Antimicrobial peptide Ac472-C4S/C12S
HNTSFLTWFTTSL

SEQ ID NO: 133 - protein, 33 aa, artificial
  Antimicrobial peptides Ac474, Ac475 and Ac476 in phage Ac6d31/S
RSQARRDFWLDSRRFWLYFRQLFIHNPKTPITP

SEQ ID NO: 134 - protein, 13 aa, artificial
  Consensus sequence for Ac474, Ac475 and Ac476
DSRRFWLYFRQLF

SEQ ID NO: 135 - protein, 18 aa, artificial
  Consensus sequence for Ac474 and Ac475
RDFWLDSRRFWLYFRQLF

SEQ ID NO: 136 - protein, 18 aa, artificial
  Consensus sequence for Ac475 and Ac476
DSRRFWLYFRQLFIHNPK

SEQ ID NO: 137 - protein, 23 aa, artificial
  Antimicrobial peptide Ac474
RSQARRDFWLDSRRFWLYFRQLF

SEQ ID NO: 138 - protein, 23 aa, artificial
  Antimicrobial peptide Ac475
RDFWLDSRRFWLYFRQLFIHNPK

SEQ ID NO: 139 - protein, 23 aa, artificial
  Antimicrobial peptide Ac476
DSRRFWLYFRQLFIHNPKTPITP

SEQ ID NO: 140 - protein, 13 aa, artificial
  Antimicrobial peptide Ac496
WCSRRRFLARCWR

SEQ ID NO: 141 - protein, 23 aa, artificial
  Antimicrobial peptide Ac315 (retroinverso analog of Ac5)
YRLLRKTTFCLKTSLTKPFHFYP

SEQ ID NO: 142 - protein, 19 aa, artificial
  Retroinverso analog of peptide Ac8
QQVMELRRLFTLRAHKHTA

SEQ ID NO: 143 - protein, 23 aa, artificial
  Antimicrobial peptide Ac205 (retroinverso analog of Ac13)
RRLARIKSLLQRCVAFFSHNYRV

SEQ ID NO: 144 - protein, 23 aa, artificial
  Retroinverso analog of peptide Ac14
RRLARIKSLLQRSVAFFSHNYRV

SEQ ID NO: 145 - protein, 23 aa, artificial
  Retroinverso analog of peptide Ac17
EILRIIYKYIGSQKGGVRRCERV

SEQ ID NO: 146 - protein, 23 aa, artificial
  Retroinverso analog of peptide Ac35
LRLCISGSLRGVRHTPVKGQYKK

SEQ ID NO: 147 - protein, 23 aa, artificial
  Retroinverso analog of peptide Ac44
ASPPPPPPTPSTTPPTNPTTCPH

SEQ ID NO: 148 - protein, 23 aa, artificial
  Retroinverso analog of peptide Ac53
KGPTHRHHRRRTGEHLKRPLPTQ

SEQ ID NO: 149 - protein, 23 aa, artificial
  Retroinverso analog of peptide Ac59
HVKKPVFFRRCTHLFRTPMAVRW

SEQ ID NO: 150 - protein, 23 aa, artificial
  Retroinverso analog of peptide Ac67
GAWRLACSASRGRGSSRGPPSRP

SEQ ID NO: 151 - protein, 22 aa, artificial
  Retroinverso analog of peptide Ac193
AMSAKSRFFSYRSSPFYRSTHP

SEQ ID NO: 152 - protein, 23 aa, artificial
  Retroinverso analog of peptide Ac195
TNKRYDKYVNLYRGAQRMFWVPI

SEQ ID NO: 153 - protein, 23 aa, artificial
  Retroinverso analog of peptide Ac322
CFKSRKLNKPLCFFGTRNFFPIP

SEQ ID NO: 154 - protein, 23 aa, artificial
  Retroinverso analog of peptide Ac322-C1S/C12S
SFKSRKLNKPLSFFGTRNFFPIP

SEQ ID NO: 155 - protein, 23 aa, artificial
  Retroinverso analog of peptide Ac328
CCRDSLRRLFGSPWVPRVACRSS

SEQ ID NO: 156 - protein, 23 aa, artificial
  Retroinverso analog of peptide Ac328- C4S/C22S/C23S
SSRDSLRRLFGSPWVPRVASRSS

SEQ ID NO: 157 - protein, 23 aa, artificial
  Retroinverso analog of peptide Ac330
VLRGPVKRMLKKTTAFYGCHPGG

SEQ ID NO: 158 - protein, 23 aa, artificial
  Retroinverso analog of peptide Ac339
YASIQRCIRRLDERLRGSFTLVG

SEQ ID NO: 159 - protein, 23 aa, artificial
  Retroinverso analog of peptide Ac370
PRRGCECHRLSLRRWAVGSLRGM

SEQ ID NO: 160 - protein, 23 aa, artificial
  Retroinverso analog of peptide Ac370-C17S/C19S
PRRGSESHRLSLRRWAVGSLRGM

SEQ ID NO: 161 - protein, 23 aa, artificial
  Retroinverso analog of peptide Ac378
GRCKSLTNCARWRWGAGSKGLDS

SEQ ID NO: 162 - protein, 23 aa, artificial
  Retroinverso analog of peptide Ac378-C15S/C21S
GRSKSLTNSARWRWGAGSKGLDS

SEQ ID NO: 163 - protein, 23 aa, artificial
  Retroinverso analog of peptide Ac389
PRRRRSRRAMSCIWRSRRSRRYG

SEQ ID NO: 164 - protein, 19 aa, artificial
  Retroinverso analog of peptide Ac431
LRFRRRIHSLCCRMTPQHC

SEQ ID NO: 165 - protein, 19 aa, artificial
  Retroinverso analog of peptide Ac431- C1S/C8S/C9S
LRFRRRIHSLSSRMTPQHS

SEQ ID NO: 166 - protein, 23 aa, artificial
  Retroinverso analog of peptide Ac436
KRWNRWESRLIQRVVPPMRNVGW

SEQ ID NO: 167 - protein, 23 aa, artificial
  Retroinverso analog of peptide Ac476
PTIPTKPNHIFLQRFYLWFRRSD

SEQ ID NO: 168 - protein, 7 aa, Enterococcus faecalis
LVTLVFV

SEQ ID NO: 169 - protein, 16 aa, Streptococcus mutans
KNLRIIRKGIHIIKKY

SEQ ID NO: 170 - protein, 10 aa, artificial
  HIV-1 tat basic region
GRKKRRQRRR

SEQ ID NO: 171 - protein, 16 aa, artificial
  Antennapedia protein transduction domain
RQIKIWFQNRRMKWKK

SEQ ID NO: 172 - protein, 16 aa, artificial
  SRCRP2
QGRVEVLYRGSWGTVC

SEQ ID NO: 173 - protein, 15 aa, artificial
  hi3/17
KQRTSIRATEGCLPS

SEQ ID NO: 174 - protein, 17 aa, artificial
  Tachyplesin1
KWCFRVCYRGICYRRCR

SEQ ID NO: 175 - protein, 23 aa, artificial
  Magainin-2
GIGKFLHSAKKFGKAFVGEIMNS

SEQ ID NO: 176 - protein, 23 aa, artificial
  Brevinin1EB
VIPFVASVAAEMQHVYCAASRKC

SEQ ID NO: 177 - protein, 13 aa, artificial
  Aurenin 1.1
GLFDIIKKIAESI

(19) United States
(12) Patent Application Publication
(10) Pub. No.: US 2011/0262508 A1
(43) Pub. Date: Oct. 27, 2011

(54) ANTIMICROBIAL COMPOSITIONS, FORMULATIONS AND USES THEREOF

(76) Inventors: Paul Michael Watt, Nedlands (AU); Wayne Thomas, Nedlands (AU); Tatjana Katharina Heinrich, Brentwood (AU)

(21) Appl. No.: 12/741,776

(22) PCT Filed: Nov. 7, 2008
(86) PCT No.: PCT/AU2008/001662

§ 371 Date: Jul. 15, 2011

Related U.S. Application Data
(60) Provisional application No. 60/986,179, filed on Nov. 7, 2007.

(51) Int. Cl. A61K 38/10; A01N 37/18; C07K 1/00; A61P 31/04; A61K 38/16; A01N 25/00; A01P 1/00; C07K 14/00; C07K 7/08

(52) U.S. Cl. 424/405; 530/350; 530/333; 530/327; 514/2.8; 530/326

(57) ABSTRACT

The present invention provides antimicrobial peptides and analogs thereof that are cytostatic or cytotoxic toward at least one gram-negative bacterium of the genus Acinetobacter including laboratory or clinical isolates of A. baumannii with low or non-detectable cytotoxicity against mammalian cells. Optionally, the antimicrobial peptides and analogs thereof are also cytotoxic or cytostatic against other unrelated bacteria such as Staphylococcus aureus.

### RELATED APPLICATION DATA

This application claims priority from U.S. Ser. No. 60/986,179 filed Nov. 7, 2007, the contents of which are incorporated herein in their entirety.

### FIELD OF THE INVENTION

The present invention relates to antimicrobial peptides and analogs and derivatives thereof, formulations comprising same and uses thereof.

1. Background of the Invention

Antibiotic resistance to pathogenic bacteria, including gram-negative and gram-positive strains, is a major problem in the pharmaceutical industry as the emergence of resistant strains outpaces the development of new antibacterial drugs. Gram-negative pathogens e.g., belonging to the families Enterobacteriaceae, Pseudomonas, Acinetobacter and Stenotrophomonas, are particularly problematic in the hospital environment. Resistant strains may differ in virulence, however they generally present natural or intrinsic resistance, and/or a capacity to acquire resistance rapidly, a factor leading to the emergence of resistant strains in hospital environments. The development of resistance poses various problems: a therapeutic problem in prescribing an active antibiotic that does not select for resistant strains; a microbiological problem in detecting specific resistance and/or mechanisms of resistance; and a public health problem in minimizing spread of multi-resistant bacteria.

2. Description of the Related Art

Acinetobacter species, especially those of the A. calcoaceticus-A. baumannii complex, include A. calcoaceticus, A. baumannii, Genomic species 3, Genomic species 13TU, A. haemolyticus, A. junii, Genomic species 6, A. johnsonii, A. lwoffii, Genomic species 10, Genomic species 11, A. radioresistens, Genomic species 14TU, Genomic species 15TU, Genomic species 14BJ, Genomic species 15BJ, Genomic species 16, Genomic species 17, A. venetianus, A. ursingii and A. schindleri. Of these, A. baumannii is an example of a gram-negative bacterium that is particularly problematic by virtue of being multidrug-resistant, nosocomial and community-acquired pathogen. A. baumannii and A. lwoffii are known to cause nosocomial infections in ventilated patients. A. baumannii causes hospital-acquired pneumonia, bloodstream infection, surgical site infection, and urinary tract infection. A. baumannii has become a problem in intensive care and burns units in hospitals, in addition to being the cause of numerous infections in areas affected by natural disasters or wars (Wilson et al., Am. J. Infect. control, 32: 342-344, 2004; Abbot Nature 436: 758, 2005; and Zapor and Moran Curr. Opin. Infect. Dis, 18: 395-399, 2005). One reason for the recent emergence of A. baumannii as a pathogen is that this bacterium is naturally resistant to many currently used antimicrobial compounds.

Pseudomonas species, e.g., Pseudomonas aeruginosa, are also gram-negative bacteria that are problematic by virtue of being multi-drug resistant and invasive. For example, P. aeruginosa causes a wide range of severe infections which may cause morbidity in immune-compromized subjects e.g., caused by HIV infection, chemotherapy, or immunosuppressive therapy. Furthermore, P. aeruginosa causes serious infections of the lower respiratory tract, the urinary tract, and wounds in younger and older hospitalized ill patients, including those suffering from cystic fibrosis. The incidence of P. aeruginosa infection among intensive care unit patients is increasing.

The Enterobacteriaceae are a large group of gram-negative rods of the gastrointestinal tract, including the genera Escherichia, Shigella, Salmonella, Enterobacter, Klebsiella, Serratia, and Proteus. Gram-negative bacteria of the Enterobacteriaceae family are important causes of urinary tract infections (UTIs), bloodstream infections, hospital-and healthcare-associated pneumonias, and various intra-abdominal infections. Within this family, Escherichia coli is a frequent cause of UTIs, Klebsiella spp and Enterobacter spp are important causes of pneumonia, and all of the Enterobacteriaceae have been implicated in bloodstream infections and in peritonitis, cholangitis, and other intra-abdominal infections. Additionally, organisms such as Salmonella spp produce gastroenteritis and subsequently, in some patients, invasive infection. Emerging resistance in Enterobacteriaceae is a significant problem that requires immediate attention. For example, resistance related to production of extended-spectrum β-lactamases (ESBLs), including resistance to cephalosporins and ciprofloxacin in Escherichia coli and Klebsiella pneumoniae, is a particular problem in the handling of Enterobacteriaceae infections.

Stenotrophomonas, e.g., S. maltophilia, are gram-negative aerobic bacilli widely distributed in natural and human environments, that are now known to be responsible for nosocomial infections such as bacteremia, pneumonia, urinary tract infections, skin and soft tissue infections, ocular infections and meningitis. For example, S. matophilia is generally considered a risk factor for malignancy, chronic respiratory disease such as cystic fibrosis, and infective endocarditis. S. maltophilia endocarditis is often hospital-acquired and related to an indwelling catheter infection, and has a high mortality rate that is most likely related to the intrinsic resistance of nosocomial bloodstream infections to commonly prescribed antibiotics.

Antimicrobial Compounds

The value of antimicrobial compounds to the pharmaceutical and animal health industry during the past 50 years is undisputed. Since the discovery of penicillin and other classes of antibiotics the rate of mortality caused by microorganism infection has fallen dramatically (Rachakonda et al., Curr. Med. Chem., 11: 775-793, 2004). This demand for antimicrobial compounds has lead to pharmaceutical companies focussing on the development of new antimicrobial compounds, resulting in the development and marketing of over 200 antimicrobial compounds in the past 50 years (Overbye and Barrett Drug Discovery Today, 10: 45-52, 2005). Generally, these antimicrobial compounds fall into two broad categories, micro-biostatic compounds and micro-biocidal compounds. Microbio-static compounds reduce or prevent growth of a microorganism, e.g., by inhibiting protein synthesis, DNA synthesis or cellular metabolism. Exemplary microbiostatic compounds include the tetracyclines, sulphonamides and spectinomycin. Microbicidal compounds kill microorganisms, e.g., by inhibiting cell wall synthesis leading to cell lysis. Exemplary microbio-static compounds include penicillin and cephalosporins.

Antimicrobial compounds suitable for therapeutic and/or prophylactic applications are generally stable such that they are capable of exerting a biological effect in vivo and/or have little or no toxicity when administered to a subject and/or have a suitable spectrum of activity, e.g., broad spectrum for a first line treatment of a variety of microorganisms or narrow spectrum for the specific treatment of one or more microorganisms, e.g., microorganisms resistant to traditional antimicrobial compounds.

Despite the focus by pharmaceutical companies on new antimicrobial compounds, during the past 30 years the only structurally new compounds approved for therapeutic applications are linezeloid and daptomycin. The remaining compounds produced are analogs of pre-existing antimicrobial agents. In fact, the majority of antimicrobial agents produced in the past fifty years are based on one of twelve core scaffolds.

The pharmaceutical industry's failure to identify new antimicrobial compounds has also led to a decline in the number of compounds approved for clinical use. In this respect, between 1983 and 2001 the Food and Drug Administration in USA approved 47 new antimicrobial compounds for therapeutic use in humans. However, between 1998 and 2005 only nine new antimicrobial agents were approved for clinical use in humans (Overbye and Barrett supra)

In an effort to identify new classes of antimicrobial compounds, the pharmaceutical and biotechnology industries have turned to antimicrobial peptides. This is because these peptides provide a greater range of structural diversity than is available in traditional antibiotic compounds comprising about fourteen core scaffolds. For example, the structure of a peptide may be dramatically changed by altering even a single amino acid within the sequence of the peptide. Accordingly, the number of possible structures within a peptide is theoretically enormous.

However, research to identify an antimicrobial peptides has focused on naturally-occurring antimicrobial peptides. Whilst peptides per se have a diverse repertoire of structures, evolution has limited the structure and function of naturally-occurring antimicrobial peptides. For example, peptides in a specific organism will only have evolved to be effective against microorganisms that infect that organism, and to be non-toxic to the organism that expresses that peptide. Accordingly, a naturally-occurring antimicrobial peptide from a non-human organism may be ineffective against a microorganism that infects a human and/or may be toxic to a human, e.g., as discussed supra.

To date there has been some success in identifying antimicrobial peptides from natural sources. However, many of these peptides suffer from one or more disadvantages.

For example, the antimicrobial peptide polymyxin B requires cyclization for stabilization. Moreover, this peptide has been shown to cause nephrotoxicity, neurotoxicity and fever when used at clinically-effective concentrations (Ostronoff et al., Int. J. Infect. Dis, 10: 339-340, 2006).

Temporin L isolated from the European red frog Rana temporaria, is toxic to human cells including erythrocytes, Rinaldi et al., Biochem J. 368: 91-100, 2002. Bovine Myeloid Antimicrobial Peptides (BMAPs) have also been shown to be toxic to cultured blood cells and blood cell-derived cell lines (Risso et al., Cell Immunol., 189: 107-115, 1998). Similarly, BombininH2 has also been shown to cause hemolysis (Csordas and Michl Monatsh Chem 101: 182-189, 1970). Accordingly, these peptides are ineffective for any form of therapy in which the peptide contacts blood cells in a subject, e.g., by intravenous administration.

Other naturally-occurring antimicrobial peptides are derived from toxins, making them undesirable for therapeutic or prophylactic use. For example, the bee venom peptide, melittin forms channel-like structures in biological membranes generally and causes hemolysis, cytolysis, membrane depolarization, activation of tissue phospholipase C and involuntary muscle contractions.

Accordingly, there remains a clear need in the art to identify antimicrobial peptides having improved activity, e.g., having reduced toxicity and/or having suitable spectra for clinical applications and/or having a high level of antimicrobial activity and/or having a stable structure. Naturally, alternative approaches to identifying antimicrobial peptides will be desirable to identify peptides having such improved activity.

### SUMMARY OF THE INVENTION

### 1. Introduction

In work leading up to the present invention, the inventors sought to exploit the diverse repertoire of peptide domains encoded by naturally-occurring nucleic acids that do not encode antimicrobial peptides in their native environment as antimicrobials. Such peptide domains have evolved to stable structures making them suitable for in vivo application without requiring complex modifications to maintain their stability. Moreover, since peptide domains have not been subject to selective pressure to form structures that possess inherent antimicrobial activity e.g., as predicted from the activities of the proteins from which they are derived, such protein domains provide a rich and diverse source of antimicrobial compounds potentially having improved activity, e.g., having reduced toxicity and/or having broad or narrow spectrum of activity and/or having a high level of antimicrobial activity and/or having a stable structure.

To identify antimicrobial peptides, the inventors sought to identify and/or isolate peptides that are bacteriostatic or bactericidal against bacteria that are problematic from the point of view of being resistant to conventional antibiotic compounds. More particularly, the inventors sought to isolate bacteriostatic and/or bactericidal peptide compositions effective against gram-negative bacteria e.g., bacteria belonging to a family selected from the group consisting of Enterobacteriaceae, Pseudomonas, Acinetobacter and Stenotrophomonas.

In one example, of the present invention, the inventors screened a phage display library of peptides encoded by genomic DNA fragments isolated from evolutionary diverse microorganisms having substantially sequenced genomes to identify peptides capable of binding to a target gram-negative bacterium e.g., Acinetobacter such as A. baumannii. In one example, phage were also selected base on their low binding affinities for non-target bacteria to thereby exclude those peptides binding to shared epitopes between target and non-target bacteria. For example, the selection process of one example preadsorbed phage to non-target gram-positive bacteria e.g., Staphylococcus aureus and/or non-target gram-negative bacteria of low virulence e.g., Pasteurella pneumotropica or low-virulence A. lwoffii. As exemplified herein, this approach has resulted in the isolation of a large number of phage clones that bound to Acinetobacter spp. including A. baumannii.

Smaller peptides were identified within the cloned phage fragments e.g., that spanned regions of predicted secondary structure. The inventors reasoned that such peptides may comprise stable structures when administered such that their turnover rates are sufficiently low to enhance efficacy e.g., by virtue of being derived from peptide domains or protein folds or other stable secondary structures or assemblies of secondary structures. Peptide sets comprising overlapping sequences were then produced and tested for efficacy against target bacterium.

Analogs of these synthetic peptides were also tested, including peptides modified by substitution of multiple cysteine residues for serine residues to prevent intramolecular disulfide bridge formation and/or by the inclusion of D-amino acids and/or by inclusion of C-terminal amidated residues and/or by formation of retroinverso analogs.

The cytostatic activity and/or cytotoxicity of peptides and their analogs against a target bacterium and non-target bacterium were determined e.g., in liquid culture assay. For example, the inventors determined the ability of an isolated peptide or analog thereof to inhibit or reduce growth of A. baumannii and/or to kill A. baumannii in liquid culture assays. The inventors also determined the ability of certain peptides to reduce or prevent growth of other gram-negative bacteria, e.g., Pseudomonas aeruginosa, Escherichia coli, Salmonella typhimurium and Pasteurella pneumotropica, and gram-positive bacteria, e.g., Staphylococcus aureus. Peptides were classified according to their ability to kill or inhibit growth of one or more Acinetobacter spp., and optionally S. aureus.

The inventors also determined the time required for certain peptides to exert bactericidal activity, preferably when used at their MIC, and classified peptides according to whether or not the cytotoxic effect was “Rapid” i.e., cell death in less than 30 mins, and preferably less than 10 mins or less than 5 mins; “Intermediate” i.e., cell death in about 30 mins to about 60 mins; or “Slow” i.e., cell death in more than about 60 mins and up to about 240 mins.

The inventors also determined safety of certain peptides in art-recognized animal models of infection with a target bacterium, for example by determining hemolysis of red blood cells (RBC), and characterized the antimicrobial peptides according to their minimum haemolytic concentration i.e., a MHC₁₀ value i.e., a minimum peptide concentration that produces 10% lysis of RBC. Peptides that did not induce apparent RBC lysis i.e., less than 0.6% apparent hemolysis at 100 μM peptide concentration, or had very low haemolytic activity i.e., less than 5% apparent hemolysis at 100 μM peptide concentration, were selected.

Cytotoxicity of certain peptides was also determined in a human T-cell line. Jurkat cells were cultured in the presence and absence of peptides and their metabolic functions as measured by Celltiter-blue dye staining were determined. Data were consistent with ranking of peptides by their cytotoxicities against Jurkat cells.

The inventor also show that peptides of the present invention can have efficacy in vivo in an animal model of A. baumannii lung infection, by demonstrating that peptides reduce cell counts in lung exudates following treatment e.g., by intravenous injection.

### 2. Specific Embodiments

The scope of the invention will be apparent from the claims as filed with the application, following the examples, which are hereby incorporated by reference into the description. The scope of the invention will also be apparent from the following description of specific examples.

In one example, the present invention provides an antimicrobial peptide or analog or derivative thereof having cytotoxicity against at least one gram-negative bacterium of the genus Acenitobacter or that is or capable of reducing or preventing growth of at least one bacterium of the genus Acenitobacter e.g., any one or more of SEQ ID NOs: 1-168, and more particularly peptides designated Ac3, Ac5, Ac8, Ac13, Ac14, Ac15, Ac16, Ac17, Ac19, Ac35, Ac38, Ac39, Ac40, Ac41, Ac44, Ac46, Ac47, Ac53, Ac59, Ac66, Ac67, Ac68, Ac96, Ac99, Ac101, Ac102, Ac116, Ac173, Ac175, Ac193, Ac195, Ac228, Ac259, Ac322, Ac328, Ac330, Ac339, Ac370, Ac378, Ac389, Ac431, Ac436, Ac476 and Ac472, retro inverted forms of Ac5 and Ac13, and any alpha-amidated analogs thereof, analogs having multiple cysteine residues substituted for serines and retroinverso analogs. For example, the antimicrobial peptide or analog or derivative thereof is at least cytotoxic against a laboratory isolate or clinical isolate of A. baumannii or at least reduces or prevents growth of a laboratory isolate or clinical isolate of A. baumannii e.g., any one or more of the peptides presented in Table 6 hereof having detectable activity against A. baumannii and any one of the peptides shown in Tables 7-8 hereof having a MIC value against A. baumannii of about 50 μM or less, preferably about 25 μM or less e.g., Ac3, Ac5, Ac8, Ac14, Ac15, Ac16, Ac17, Ac19, Ac35, Ac38, Ac39, Ac40 and Ac59, retro inverted forms of Ac5 and Ac13, and any alpha-amidated analogs thereof, analogs having multiple cysteine residues substituted for serines and retroinverso analogs. In another example, the antimicrobial peptide or analog or derivative thereof is cytotoxic or cytostatic toward A. lwoffii. In yet another example, the antimicrobial peptide or analog or derivative thereof is at least cytotoxic against one or more of ATCC Accession No. 19606, ATCC Accession No. 17903 and ATCC Accession No. 19004, or at least reduces or prevents growth one or more of ATCC Accession No. 19606, ATCC Accession No. 17903 and ATCC Accession No. 19004 e.g., any one or more of the peptides presented in Tables 6-8 hereof having detectable activity against one or more of ATCC Accession No. 19606, ATCC Accession No. 17903 and ATCC Accession No. 19004. In this context, the term “detectable activity” includes more than about 95% inhibition of growth at a peptide concentration of about 50 μM or less.

In another example, the antimicrobial peptide or analog or derivative thereof has a minimum inhibitory concentration (MIC) toward A. baumannii of 25 μM or less, or alternatively, a MIC toward A. baumannii of 25 μM or less, including less than 2.5 μM or less than 5 μM or less than 10 μM or less than 15 μM or less than 20 μM peptide concentration. Exemplary peptides include peptides designated Ac5, Ac8, Ac13, Ac14, Ac16, Ac17, Ac35, Ac38, Ac39, Ac40, Ac44, Ac53, Ac59, Ac193, Ac195, Ac228, Ac259, Ac319, Ac322, Ac323, Ac327, Ac328, Ac330, Ac339, Ac370, Ac378, Ac389, Ac431, Ac436, Ac469, Ac472, Ac474, Ac475 and Ac476, retro inverted forms of Ac5 and Ac13, and any alpha-amidated analogs thereof, analogs having multiple cysteine residues substituted for serines and retroinverso analogs.

Alternatively, or preferably, the present invention provides an antimicrobial peptide or analog or derivative thereof having reduced toxicity to mammalian cells. As used herein, the term “reduced toxicity” shall be taken to mean that a peptide or analog or derivative thereof does not induce one or more adverse response(s) in a subject or in a cell, tissue or organ of a subject to which it is administered, e.g., a peptide or analog or derivative dies not cause dysfunction of an organ or a system of organs or cause cell death. For example, an antimicrobial peptide of the invention or analog or derivative thereof is not nephrotoxic and/or is not neurotoxic and/or does not cause rhabdomyolysis and/or does not cause seizures and/or does not cause cardiac arrhythmia and/or does not have a myelosuppressive effect and/or does not cause diarrhea and/or does not cause anaphylaxis and/or does not cause significant levels of hemolysis, which may result in hemolytic anemia. Methods for determining toxicity of a peptide or analog or derivative thereof will be apparent to the skilled artisan and include, for example, contacting a sample of red blood cells with the peptide analog or derivative and determining the level of hemolysis caused by the peptide analog or derivative.

An antimicrobial peptide or analog or derivative thereof of the present invention may have low or non-detectable cytotoxicity against mammalian cells e.g., red blood cells or T-lymphocytes such as Jurkat cells. Exemplary antimicrobial peptides, analogs and derivatives within the scope of the invention induce cytotoxicity in less than 10% of a culture of red blood cells or T-lymphocytes at concentrations equal to or greater than the minimum inhibitory concentration used in the prophylactic or therapeutic treatment of infection by one or more bacterial agents e.g., 1 μM or greater concentration, alternatively 10 μM or greater concentration, alternatively 25 μM or greater concentration, alternatively 50 μM or greater concentration, a alternatively 100 μM or greater concentration. In accordance with this example, the antimicrobial peptide or analog or derivative thereof may, for example, induce cytotoxicity in less than 10% of a culture of red blood cells or T-lymphocytes at a concentration of 100 μM or greater, and preferably induce cytotoxicity in less than 5% of a culture of red blood cells or T-lymphocytes at a concentration of 100 μM or greater concentration, and more preferably induces cytotoxicity in less than 1% of a culture of red blood cells or T-lymphocytes at a concentration of 100 μM or greater concentration. Even more preferably, the antimicrobial peptide or analog or derivative thereof does not induce detectable cytotoxicity in a culture of red blood cells or T-lymphocytes at a concentration of 100 μM or greater concentration. Exemplary peptides within these contexts include e.g., Ac5, Ac8, Ac14, Ac17, Ac38, Ac44, Ac53, Ac59, Ac67, Ac193, Ac195, Ac319, retro inverted forms thereof, alpha-amidated analogs thereof, and analogs having multiple cysteine residues substituted for serines and retroinverso analogs.

Alternatively, or in addition, an antimicrobial peptide or analog or derivative thereof of the present invention has weak cytotoxicity or is weakly cytostatic against Escherichia coli, or has weak cytotoxicity or is weakly cytostatic against one or more of Escherichia coli strain BL21, S. typhimurium strain AroA, P. aeruginosa or P. pneumotropica. By weakly cytotoxic or weakly cytostatic is meant that the peptide, analog or derivative has a MIC toward said bacterium of more than about 25 μM and preferably more than about 50 μM, and even more preferably more than about 100 μM peptide concentration.

Alternatively, or in addition, an antimicrobial peptide or analog or derivative thereof of the present invention is cytotoxic against a plurality of Acenitobacter spp. or cytostatic against a plurality of Acenitobacter spp. i.e., it reduces or prevents growth of a plurality of Acenitobacter spp, e.g., Ac3, Ac5, Ac8, Ac14, Ac15, Ac16, Ac17, Ac19, Ac35, Ac38, Ac39, Ac40, Ac59, retro inverted forms thereof, alpha-amidated analogs thereof, and analogs having multiple cysteine residues substituted for serines and retroinverso analogs. In accordance with this example, a MIC of the peptide, analog or derivative toward each of the plurality need not be the same, however will generally be in the order of 50 μM or less and more typically 25 μM or less, including less than 2.5 μM or less than 5 μM or less than 10 μM or less than 15 μM or less than 20 μM peptide concentration. In one example, the plurality at least comprises a laboratory isolate or clinical isolate of A. baumannii. In another example, the plurality of Acenitobacter sp. comprises a plurality of laboratory and/or clinical isolates of A. baumannii. In another example, the antimicrobial peptide or analog or derivative thereof has cytotoxic activity or bacteriostatic activity against a plurality of Acenitobacter spp. and against S. aureus. In another example, the plurality of Acenitobacter spp. comprise one or more laboratory and/or clinical isolates of A. baumannii and one or both of ATCC Accession No. 17903 Acinetobacter spp. ATCC 19004. In another example, the plurality of Acenitobacter spp. comprise ATCC Accession No. 19004.

Alternatively, or in addition, an antimicrobial peptide or analog or derivative thereof of the present invention has a broad spectrum of antimicrobial activity. As used herein, the term “broad spectrum” shall be taken to mean that an antimicrobial peptide is capable of reducing or preventing growth of or killing a plurality of unrelated microorganisms. For example, the antimicrobial peptide, analog or derivative of the present invention is optionally cytotoxic or cytostatic against S. aureus. The term “microorganism” as used herein includes any microscopic organism and, preferably, a pathogenic microscopic organism e.g., a bacterium, an archaebacterium, a virus, a yeast, a fungus or a protist, e.g., cryptosporidium. Exemplary broad spectrum peptides include peptides designated Ac3, Ac5, Ac8, Ac19, Ac38, Ac39, Ac40, Ac41, Ac44, Ac46, Ac47, Ac53, Ac66, Ac67, Ac96, Ac99, Ac101, Ac102, retro inverted forms thereof, alpha-amidated analogs thereof, and analogs having multiple cysteine residues substituted for serines and retroinverso analogs.

In one example, such a broad spectrum antimicrobial peptide or analog or derivative thereof is capable of reducing or preventing growth of S. aureus or killing S. aureus e.g., Ac3, Ac5, Ac8, Ac19, Ac38, Ac39, Ac40, Ac41, Ac44, Ac46, Ac47, Ac53, Ac66, Ac67, Ac96, Ac99, Ac101, Ac102, retro inverted forms thereof, alpha-amidated analogs thereof, and analogs having multiple cysteine residues substituted for serines and retroinverso analogs. Such an antimicrobial peptide or analog or derivative thereof is useful for the treatment of, for example, methicillin-resistant S. aureus.

Alternatively, or preferably, the present invention provides an antimicrobial peptide or analog or derivative thereof that preferentially or selectively reduces or prevents growth of one species of microorganism or a specific group of microorganisms or kills one species microorganism or a specific group of microorganisms. By “preferentially” is meant that a peptide or analog or derivative reduces or prevents growth of one species of microorganism or one group of microorganisms to a greater extent or higher level than it reduces or prevents growth of another species of microorganism or another group of microorganisms. However, the term “preferentially” is not to be understood that the peptide or analog or derivative thereof only reduces or prevents growth of one species of microorganism or one species of microorganism. By “selectively” is meant that a peptide or analog or derivative reduces or prevents growth of one species of microorganisms or one group of microorganisms and does not detectably reduces or prevents growth of any other species of microorganisms or any other group of microorganisms. In one example, the present invention provides an antimicrobial peptide or analog or derivative thereof that preferentially or selectively reduces or prevents growth of a bacterium of the genus Acenitobacter, e.g., A. baumannii or selectively or preferentially kills a bacterium of the genus Acenitobacter, e.g., A. baumannii. In another example, the present invention provides an antimicrobial peptide or analog or derivative thereof that preferentially or selectively reduces or prevents growth of a bacterium of the genus Acenitobacter, e.g., A. baumannii and a bacterium of the genus Pasteurella, e.g., P. pneumotropica or selectively or preferentially kills a bacterium of the genus Acenitobacter, e.g., A. baumannii and kills a bacterium of the genus Pasteurella e.g., P. pneumotropica.

Alternatively, or preferably, the present invention provides an antimicrobial peptide or analog or derivative thereof having improved activity compared to a known antimicrobial peptide. As used herein, the term “improved activity” shall be taken to mean that a peptide or analog or derivative reduces or prevents growth of a microorganism or kills a microorganism at a lower concentration than a known antimicrobial peptide, e.g., has a lower minimum inhibitory concentration (MIC). For example, the antimicrobial peptide or analog or derivative reduces or prevents growth of a bacterium of the genus Acenitobacter and/or a bacterium of the genus Staphylococcus or kills a bacterium of the genus Acenitobacter and/or a bacterium of the genus Staphylococcus at a lower concentration than a known antimicrobial peptide. In one example, the present invention provides an antimicrobial peptide or analog or derivative thereof having improved activity compared to a scavenger receptor cysteine rich peptide (SRCRP2) comprising a sequence set forth in SEQ ID NO: 172 and/or a hi3/17 peptide comprising a sequence set forth in SEQ ID NO: 173 and/or a Brevinin 1 EB peptide comprising a sequence set forth in SEQ ID NO: 176 and/or an Aurein 1.1 peptide comprising a sequence set forth in SEQ ID NO: 177. Exemplary peptides having such improved activity comprise a sequence set forth in any one of SEQ ID NOs; 1-167.

Alternatively, the present invention provides an antimicrobial peptide or analog or derivative thereof, said peptide or analog or derivative being selected individually or collectively from the group consisting of:
(i) a peptide comprising a sequence set forth in any one of SEQ ID NOs: 1-167;
(ii) an antimicrobial peptide that is a variant of (i) having at least about 70% or 80% or 90% or 95% sequence identity thereto and comprising a sequence that differs from a sequence set forth in (i) or (ii) by one or more conservative amino acid substitutions, said variant being capable of reducing or preventing growth of one or more microorganisms and/or killing one or more microorganisms;
(iii) a peptide comprising a fragment of the sequence of (i) or (ii) said fragment being capable of reducing or preventing growth of one or more microorganisms and/or killing one or more microorganisms;
(iv) an analog of any one of (i) to (iii) selected from the group consisting of (a) the sequence of any one of (i) to (iii) comprising one or more naturally-occurring amino acid substitutions; (b) the sequence of any one of (i) to (iii) comprising one or more non-naturally-occurring amino acid analogs; (c) an isostere of any one of (i) to (iii); (d) a retro-peptide analog of any one of (i) to (iii); and (e) a retro-inverted peptide analog of any one of (i) to (iii).

In another example, the present invention provides an antimicrobial peptide or analog or derivative thereof, said peptide or analog or derivative being selected individually or collectively from the group consisting of:
(i) a peptide comprising a sequence set forth in any one of SEQ ID NOs: 1-140;
(ii) an antimicrobial peptide that is a variant of (i) having at least about 70% or 80% or 90% or 95% sequence identity thereto and comprising a sequence that differs from a sequence set forth in (i) or (ii) by one or more conservative amino acid substitutions, said variant being capable of reducing or preventing growth of one or more microorganisms and/or killing one or more microorganisms;
(iii) a peptide comprising a fragment of the sequence of (i) or (ii) said fragment being capable of reducing or preventing growth of one or more microorganisms and/or killing one or more microorganisms;
(iv) an analog of any one of (i) to (iii) selected from the group consisting of (a) the sequence of any one of (i) to (iii) comprising one or more naturally-occurring amino acid substitutions; (b) the sequence of any one of (i) to (iii) comprising one or more non-naturally-occurring amino acid analogs; (c) an isostere of any one of (i) to (iii); (d) a retro-peptide analog of any one of (i) to (iii); and (e) a retro-inverted peptide analog of any one of (i) to (iii).

By “individually” is meant that the invention encompasses the recited antimicrobial peptides or groups of antimicrobial peptides separately, and that, notwithstanding that individual peptides or groups of peptides may not be separately listed herein the accompanying claims may define such peptides or groups of peptides separately and divisibly from each other.

By “collectively” is meant that the invention encompasses any number or combination of the recited antimicrobial peptides or groups of antimicrobial peptides, and that, notwithstanding that such numbers or combinations of peptides or groups of peptides may not be specifically listed herein the accompanying claims may define such combinations or sub-combinations separately and divisibly from any other combination of peptides or groups of peptides.

In one example, the antimicrobial peptide or analog or derivative thereof of the present invention is capable of preventing or reducing the growth of or killing a bacterium. In one example, the antimicrobial peptide or analog or derivative thereof is capable of preventing or inhibiting the growth of or killing a Gram negative bacterium, e.g., a Gram negative bacterium belonging to a genus selected_{—} from the group consisting of Acinetobacter, Escherichia, Pseudomonas and Salmonella. Alternatively, or in addition, the antimicrobial peptide or analog or derivative thereof of the present invention is capable of preventing or inhibiting the growth of or killing a Gram positive bacterium e.g., a bacterium belonging to the genus Staphylococcus. Additional gram-negative and/or gram-positive bacteria are not to be excluded.

Preferred analogs and derivatives other than those specifically exemplified herein will comprise an amino acid sequence at least about 70% identical to an amino acid sequence set forth in any one or more of SEQ ID NOs: 1-167 or a derivative or analog thereof. Analogs and derivatives other than those specifically disclosed herein are readily produced without undue experimentation based on the teaching provided herein and/or based on the known structure/function relationships of various classes of analogs and derivatives e.g., retro inverted peptides such as set forth in SEQ ID NOs: 141-167.

As will be apparent to the skilled artisan from the description herein, it is preferable that a peptide of the invention comprises a stable structure, e.g., a peptide domain or subdomain, and is encoded by a nucleic acid that encodes a domain of a protein that does not have antimicrobial activity in its native context, or is encoded by a nucleic acid that does not encode a peptide or protein in its native context.

As used herein, the term “antimicrobial” shall be taken to mean that the peptide is capable of killing a microorganism and/or reducing or preventing growth of a microorganism. Methods for determining the antimicrobial activity of a peptide will be apparent to the skilled artisan and/or described herein. For example, the peptide is applied to or contacted to a solution in which a microorganism has been previously grown and, after a suitable period of time, the level of growth inhibition and/or cell death of the microorganism is determined.

As used herein, the term “analog” shall be taken to mean a peptide that is modified to comprise one or more naturally-occurring and/or non-naturally-occurring amino acids, provided that the peptide analog is capable of reducing or preventing growth of a microorganism or killing a microorganism. For example, the term “analog” encompasses an inhibitory peptide comprising one or more conservative amino acid changes. The term “analog” also encompasses a peptide comprising, for example, one or more D-amino acids. Such an analog has the characteristic of, for example, protease resistance.

As used herein the term “derivative” shall be taken to mean a peptide that is derived from an inhibitory peptide as described herein e.g., a fragment or processed form of the peptide. The term “derivative” also encompasses fusion proteins comprising a peptide of the invention. For example, the fusion protein comprises a label, such as, for example, an epitope, e.g., a FLAG epitope or a V5 epitope or an HA epitope. For example, the epitope is a FLAG epitope. Such a tag is useful for, for example, purifying the fusion protein.

A preferred derivative of an antimicrobial peptide of the invention has enhanced stability. For example, a cleavage site of a protease active in a subject to which a peptide is to be administered is mutated and/or deleted to produce a stable derivative of an antimicrobial peptide of the invention.

The term “derivative” also encompasses a derivatized peptide, such as, for example, a peptide modified to contain one or more-chemical moieties other than an amino acid. The chemical moiety may be linked covalently to the peptide e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound.

The present invention also provides a fusion protein comprising one or more antimicrobial peptides or analogs or derivatives thereof according to any example hereof e.g., a plurality of antimicrobial peptides or analogs or derivatives as described according to any example hereof. Such a fusion protein can comprise a plurality of the same antimicrobial peptide or analog or derivative, or a plurality of different peptides and/or analogs and/or derivatives, or both a plurality of the same and different peptides and/or analogs and/or derivatives. In the case of an antimicrobial peptide that acts by forming a multimer, e.g., to form a channel in a membrane of a microorganism, such a fusion protein can assist with formation of the channel. For example, a fusion protein may comprise one or more antimicrobial peptides or analogs or derivatives as described as described according to any example hereof and one or more antimicrobial peptides, analogs or derivatives comprising a sequence other than that set forth in any one of SEQ ID NOs: 1-167. In another example, a fusion protein may comprise one or more antimicrobial peptide, analogs or derivatives according to any example hereof conjugated to a pheromone, e.g., a pheromone produced by a microorganism, such as for targeting the antimicrobial moiety selectively or specifically to a bacterial cell. The sequence of a suitable pheromone will be apparent to the skilled artisan and/or readily derived from publicly available sequence databases and include, for example, a cCF10 pheromone from Enterococcus faecalis (SEQ ID NO: 168).

The present invention also provides a formulation comprising an effective amount of one or more antimicrobial peptides, analogs derivatives and/or fusion proteins as described according to any example hereof and a suitable carrier or excipient. For example, the formulation may be a pharmaceutical formulation suitable for administration by any one or more of a variety of routes of administration, e.g., topical administration or oral administration or ocular administration or intravenous administration or intraperitoneal administration amongst others. In another example, the formulation is for disinfecting or sterilizing a solution or a surface or for preserving a composition subject to spoilage caused by a microorganism (e.g., for preventing microbial growth on or in a food product). Such compositions may take any of a number of forms, such as, for example, a solution (e.g., a spray solution or a pharmaceutical solution, e.g., a nasal spray solution or syrup), an aerosol, a cream, a lotion, a gel or a powder. Suitable compositions will be apparent to the skilled artisan based on the description herein.

As used herein, the term “effective amount” or similar term shall be taken to mean a sufficient quantity of an antimicrobial peptide or analog or derivative of the present invention to reduce or prevent growth of a microorganism or to kill microorganisms in the context in which a formulation of the present invention is to be used. The precise amount of the stated integer will vary depending on the specific activity of the peptide, analog or derivative or formulation comprising same and/or the purpose for which the formulation is to be used. Accordingly, this term is not to be construed to limit the invention to a specific quantity, e.g., weight or concentration, unless specifically stated otherwise. Methods for assessing efficacy of any amount of a peptide or analog or derivative of the present invention in preventing or reducing growth of a microorganism or killing a microorganism will be apparent to the skilled artisan from the disclosure herein.

As used herein, the term “suitable carrier or excipient” shall be taken to mean a compound or mixture thereof that is suitable for use in a formulation for a purpose as described herein according to any embodiment. For example, a suitable carrier or excipient for use in a pharmaceutical formulation for injection into a subject will generally not cause an adverse response in a subject. A suitable carrier for excipient for use in a formulation for preserving a foodstuff will not comprise a substance toxic to a subject that will consume the foodstuff.

A carrier or excipient useful in the composition of the present invention will generally not inhibit to any significant degree a relevant biological activity of the active compound e.g., the carrier or excipient will not significantly inhibit the antimicrobial activity of an antimicrobial peptide or analog or derivative of the present invention. For example, a carrier or excipient may merely provide a buffering activity to maintain the active compound at a suitable pH to thereby exert its biological activity, e.g., phosphate buffered saline. Alternatively, or in addition, the carrier or excipient may comprise a compound that enhances the activity or half-life of the active peptide e.g., a protease inhibitor. In yet another example, the carrier or excipient may include an additional antimicrobial compound and/or an anti-inflammatory compound.

The present invention also provides a solid surface coated with or having adsorbed thereto an antimicrobial peptide, analog or derivative as described according to any example hereof. For example, the solid surface may comprise a bead or implant coated with an antimicrobial peptide or analog or derivative of the present invention, e.g., for insertion into a subject to treat a disease or disorder. Alternatively, a medical device, e.g., a prosthetic device or a catheter coated with or having adsorbed thereto an antimicrobial peptide or analog or derivative of the present invention is provided for prophylaxis or therapy. Alternatively, or in addition, a packaging for a foodstuff coated with or having adsorbed thereto an antimicrobial peptide or analog or derivative of the present invention is provided for preserving a foodstuff contained within the packaging.

In other examples, the present invention provides for use of the antimicrobial peptides, analogs and derivatives described herein in therapy and prophylaxis, and for preventing spoilage of food products. The skilled artisan will appreciate that a peptide or analog or derivative or formulation that reduces or prevents growth of a microorganism or kills a microorganism is also useful in medicine, e.g., to treat or prevent an infection. Accordingly, the present invention provides for the use of an antimicrobial peptide or analog or derivative or formulation of the present invention in medicine. The present invention also provides an antimicrobial peptide or analog or derivative or formulation of the present invention for use in medicine.

In one example, the invention provides a use of one or more antimicrobial peptides, analogs or derivatives according to any example hereof and/or one or more fusion proteins comprising said one or more antimicrobial peptides, analogs or derivatives for killing at least one gram-negative bacterium of the genus Acenitobacter or for reducing or preventing growth of at least one bacterium of the genus Acenitobacter. In another example, the invention provides a use of one or more antimicrobial peptides, analogs or derivatives according to any example hereof and/or one or more fusion proteins comprising said one or more antimicrobial peptides, analogs or derivatives in the manufacture of a composition for killing at least one gram-negative bacterium of the genus Acenitobacter or for reducing or preventing growth of at least one bacterium of the genus Acenitobacter. In another example, the invention provides a use of one or more antimicrobial peptides, analogs or derivatives according to any example hereof and/or one or more fusion proteins comprising said one or more antimicrobial peptides, analogs or derivatives in medicine. In another example, the invention provides a method for killing at least one gram-negative bacterium of the genus Acenitobacter or for reducing or preventing growth of at least one bacterium of the genus Acenitobacter, said method comprising contacting the gram-negative bacterium or a surface or composition of matter suspected of being contaminated with the gram-negative bacterium with one or more antimicrobial peptides, analogs or derivatives according to any example hereof and/or one or more fusion proteins comprising said one or more antimicrobial peptides, analogs or derivatives for a time and under conditions sufficient to kill the gram-negative bacterium or to reduce or prevent growth thereof. In another example, the invention provides a method of therapeutic or prophylactic treatment of a subject comprising administering one or more antimicrobial peptides, analogs or derivatives according to any, example hereof and/or one or more fusion proteins comprising said one or more antimicrobial peptides, analogs or derivatives to a subject in need thereof.

In another example, the invention provides a method of therapeutic or prophylactic treatment of a subject for bacterial infection, said method comprising:
(i) determining a subject suffering from a bacterial infection or at risk of developing a bacterial infection;
(ii) obtaining one or more antimicrobial peptides, analogs or derivatives according to any example hereof and/or one or more fusion proteins comprising said one or more antimicrobial peptides, analogs or derivatives and/or a formulation comprising said peptides, analogs, derivatives or fusion proteins; and
(iii) administering said peptide or analog or derivative or fusion protein or formulation to said subject for a time and under conditions sufficient to reduce or prevent bacterial infection.

In yet another example, the present invention provides a method for the prophylactic or therapeutic treatment of a bacterial infection, said method comprising:
(i) identifying a subject suffering from an infection or suspected of suffering from a bacterial infection or at risk of developing a bacterial infection; and
(ii) recommending administration of one or more antimicrobial peptides, analogs or derivatives according to any example hereof and/or one or more fusion proteins comprising said one or more antimicrobial peptides, analogs or derivatives and/or a formulation comprising said peptides, analogs, derivatives or fusion proteins sufficient to reduce or prevent bacterial infection.

In yet another example, the present invention provides a method for the prophylactic or therapeutic treatment of a bacterial infection, said method comprising administering or recommending administration of one or more antimicrobial peptides, analogs or derivatives according to any example hereof and/or one or more fusion proteins comprising said one or more antimicrobial peptides, analogs or derivatives and/or a formulation comprising said peptides, analogs, derivatives or fusion proteins sufficient to reduce or prevent bacterial infection in the subject.

For example, the medicament is for the treatment or prophylaxis of a subject suffering from an infection. As used herein, the term “subject” shall be taken to mean any animal, including a human, non-human animal, plant or insect that may be infected by a microorganism. Preferably, the subject is any animal, including a human, plant or insect that may be infected by a microorganism against which an antimicrobial peptide or analog or derivative of the invention is active. As used herein, the term “infection” shall be taken to mean the invasion, development and/or multiplication of a microorganism within or on another organism. An infection may be localized to a specific region of an organism or systemic. Infections for which a peptide, analog and/or derivative of the invention are useful for treating include any infection caused by any microorganism, e.g., bacteria and will be apparent to the skilled artisan from the disclosure herein. Preferably, an antimicrobial peptide or analog or derivative or formulation of the present invention is useful for treating an infection by a bacterium, e.g., a Gram negative bacterium or a Gram positive bacterium, or a bacterium from the genus Acinetobacter, or from the genus Staphylococcus or from the genus Salmonella or from the genus Pseudomonas or from the genus Pasteurella.

The present invention is not limited to the treatment of an infection in an animal subject. Rather, a peptide, analog and/or derivative of the present invention is also useful for, for example, treatment of a plant to thereby reduce or prevent a microbial infection therein or thereon. Accordingly, the antimicrobial peptide of the invention or analog or derivative thereof is a phytoprotective agent. In a preferred embodiment, the subject is an animal, and more preferably a mammal, e.g., a human.

The present invention also provides a method of therapeutic or prophylactic treatment of a subject comprising administering an antimicrobial peptide or analog or derivative or formulation of the present invention to a subject in need thereof. In this respect, a subject in need of treatment with a peptide, analog or derivative of the invention is, for example, a subject suffering from an infection or suspected of suffering from an infection or at risk of developing an infection

Preferably, the peptide or analog or derivative or formulation is administered under conditions sufficient for the peptide or analog or derivative to reduce or prevent growth of a microorganism and/or to kill a microorganism.

The antimicrobial peptide, analog and/or derivative of the invention can be administered to a subject by any of a variety of means, such as, for example, topical administration, nasal administration, oral administration, vaginal administration, rectal administration, intravenous administration, intraperitoneal administration, or subcutaneous administration. For example, as infectious microorganisms generally enter a mammal by way of a membrane, e.g., a mucus membrane, a peptide or analog or derivative or formulation of the invention is preferably administered in a manner suitable to contact a membrane. For example, the peptide or analog or derivative or formulation is administered by topical administration, nasal administration, oral administration, vaginal administration, rectal administration.

In the case of a systemic infection or a localised infection of a tissue or part thereof that is within a subject, a peptide or analog or derivative or formulation is administered by, for example, intravenous administration, intraperitoneal administration, or subcutaneous administration. Preferably, the peptide or analog or derivative is resistant to protease degradation to thereby increase its half-life in the subject and, as a consequence, it therapeutic/prophylactic benefit.

An antimicrobial peptide of the invention or an analog or derivative thereof can also be administered to a subject by expressing the peptide or analog or derivative in the subject. For example, the peptide or analog or derivative is expressed in a transgenic subject or is expressed by a cell administered to a subject, e.g., ex vivo therapeutic or prophylactic treatment. Methods for expressing a peptide of the invention in a cell or subject will be apparent to the skilled artisan and/or described herein.

Alternatively, the method of treatment comprises administering or recommending administration of an antimicrobial peptide or analog or derivative or formulation of the present invention to a subject previously identified as suffering from an infection or previously identified as being at risk of developing an infection.

The therapeutic method described herein is not to be limited to a single application of a peptide or composition of the invention. The present invention also contemplates repeated administration of a peptide or analog or derivative or formulation as described herein according to any embodiment e.g., to extend the period over which beneficial effects are derived.

The present invention also provides a method for prolonging the storage life of a perishable product, said method comprising:
(i) contacting a perishable product with one or more antimicrobial peptides, analogs or derivatives according any example hereof and/or one or more fusion proteins comprising said one or more antimicrobial peptides, analogs or derivatives and/or a formulation comprising said peptides, analogs, derivatives or fusion proteins for a time and under conditions sufficient to reduce or prevent growth of a microorganism and/or to kill a microorganism; and
(ii) storing the perishable product for a time that is longer than the time the product would have been stored in the absence of contact with the peptide, analog, derivative, fusion protein or formulation.

In a related example, the invention provides a use of one or more antimicrobial peptides, analogs or derivatives according to any example hereof and/or one or more fusion proteins comprising said one or more antimicrobial peptides, analogs or derivatives and/or a formulation comprising said peptides, analogs, derivatives or fusion proteins for prolonging the storage life of a perishable product.

In this respect, the perishable product is capable of being stored for a longer period of time than the same product that has not been contacted with an antimicrobial peptide or analog or derivative or formulation of the invention. The skilled artisan will be aware that such a method is useful for prolonging the storage life of, for example, a food product, e.g., meat, fruit, vegetable, dairy; a pharmaceutical composition; and/or a washing solution, e.g., saline for contact lenses. Such methods are also suitable for, for example, disinfecting a surface and/or preserving a food product and/or reducing or preventing water contamination.

Alternatively, or in addition, the method comprises applying to a surface or composition of matter suspected of comprising a microorganism a peptide or analog or derivative or formulation of the present invention for a time and under conditions sufficient to reduce or prevent growth of a microorganism and/or kill a microorganism, thereby reducing or preventing growth of a microorganism or killing a microorganism. For example, the peptide, analog or derivative of the invention is sprayed onto the surface or composition of matter. Such spray application is useful for, for example, preparing a surface for preparation of a foodstuff or sterilizing a composition of matter to be inserted into a subject. This is because spraying the peptide or analog or derivative or formulation reduces the handling of said surface or composition of matter, thereby further reducing the risk of microorganism contamination.

The present invention also provides methods for providing or producing an isolated antimicrobial peptide or analog or derivative of the invention. For example, these methods may rely upon structural information concerning the antimicrobial peptide, analog and/or derivative (e.g., the sequence of the peptide or analog or derivative or the sequence of a nucleic acid encoding the antimicrobial peptide) and/or synthesis or expression of the peptide, analog or derivative. Methods for synthesizing and/or expressing an antimicrobial peptide, analog or derivative of the invention will be apparent to the skilled artisan based on the description herein.

In one example, the invention provides a method of peptide synthesis comprising obtaining a sequence of an antimicrobial peptide or analog or derivative according to any example hereof or a fusion protein fusion protein comprising said antimicrobial peptide or analog or derivative, and synthesizing the peptide, analog, derivative or fusion protein. The method may additionally comprise providing the peptide, analog, derivative or fusion protein.

In another example, the present invention provides a method of peptide production comprising obtaining a sequence of an antimicrobial peptide or analog or derivative according to any example hereof or a fusion protein fusion protein comprising said antimicrobial peptide or analog or derivative, and performing a process of mutation or affinity maturation of the peptide, analog or derivative or otherwise altering the sequence of the peptide, analog or derivative to thereby produce a peptide having enhanced antimicrobial activity to toward one or more bacteria. The method may additionally comprise isolating the peptide having enhanced antimicrobial activity to toward one or more bacteria and/or determining a primary and/or a secondary structure for the peptide having enhanced antimicrobial activity to toward one or more bacteria and/or providing the peptide having enhanced antimicrobial activity to toward one or more bacteria or providing a primary and/or a secondary structure for the peptide having enhanced antimicrobial activity to toward one or more bacteria. It is to be understood that the present invention also extends to the direct product of this method e.g., peptide or analog or derivative isolated by the method.

The present invention also provides a method for isolating an antimicrobial peptide, said method comprising:

(i) isolating a peptide capable of binding to a microorganism, e.g., a bacterium of the genus Acenitobacter, e.g., Acenitobacter baumannii;

(ii) optionally, determining the ability of the peptide at (i) to bind to a microorganism that is different to the first microorganism; and

(iii) isolating a peptide capable of binding to at least the first miroorganism.

In one example, the method additionally comprises:

(iv) determining the ability of the peptide to reduce or prevent growth of the first microorganism or to kill the first microorganism;

(v) optionally, determining the ability of the peptide to reduce or prevent growth of a second microorganism that is different to the first microorganism; and

(vi) isolating a peptide capable of reducing or preventing growth of at least the first microorganism.

To isolate peptides capable of binding to A. baumannii, a modification of a subtractive phage display process (e.g., Bishop-Hurley et al., Antimicrob. Agents Chemother. 49: 2972-2978, 2005) was developed and employed. Briefly, a T7 phage display library is optionally preadsorbed to one or more of A. lwoffii, Pasteurella pneumotropica and Staphylococcus aureus, and those clones that do not bind are screened in a process comprising three to five rounds of screening by contacting with Acenitobacter spp., e.g., A. baumannii and/or A. lwoffii subject to the proviso that a preadsportion and screening are not conducted using the same bacterium, and the phage clones that bind to Acenitobacter spp., in each round are retained. Optionally, the clones that bind to Acenitobacter spp., in each round are purified further by contacting the clones with one or more of Pasteurella pneumotropica and Staphylococcus aureus, and retaining those clones that do not bind to one or more of Pasteurella pneumotropica and Staphylococcus aureus. Optionally, the phage lysate obtained between each round of screening is subjected to precipitation e.g., using polyethylene glycol (PEG). Optionally, a sequence of a peptide expressed by a clone that binds reproducibly to Acenitobacter spp., is determined. Optionally, a synthetic peptide is produced that comprises a sequence of a peptide expressed by a clone that binds reproducibly to Acenitobacter spp., or a variant thereof e.g., comprising D-amino acids and/or a C-terminal amidated residue or a retro inverted analog of the peptide. Optionally, the minimum inhibitory concentration (MIC) of the synthetic peptide against Acenitobacter spp. is determined, and a peptide having a MIC value of about 25 μM or less than about 25 μM against Acenitobacter spp. is retained.

The present invention also provides a method for isolating an antimicrobial peptide, said method comprising:

(i) isolating a peptide capable of binding to a first microorganism e.g., Acenitobacter baumannii;

(ii) optionally, determining the ability of the peptide at (i) to bind to a second microorganism e.g., a microorganism other than A. baumannii;

(iii) determining the ability of the peptide to reduce or prevent growth of the first microorganism e.g., A. baumannii or to kill A. baumannii;

(iv) optionally, determining the ability of the peptide to reduce or prevent growth of the second microorganism e.g., the microorganism other than A. baumannii or to kill the second microorganism e.g., the microorganism other than A. baumannii; and

(v) isolating a peptide capable of binding to at least the first microorganism e.g., A. baumannii and capable of reducing or preventing growth of at least the first microorganism e.g., A. baumannii or killing the first microorganism e.g., A. baumannii, thereby isolating an antimicrobial peptide.

In a further example of the present invention, there is provided a method of isolating a peptide capable of binding to an Acenitobacter sp. bacterium e.g., A. baumannii or A. lwoffii, said method comprising:

(i) optionally contacting a peptide preadsorbed to one or more of A. lwoffii, Pasteurella pneumotropica and Staphylococcus aureus, and those clones that do not bind are screened in a process comprising three to five rounds of screening by contacting with Acenitobacter spp., e.g., A. baumannii and/or A. lwoffii subject to the proviso that a preadsportion and screening are not conducted using the same bacterium, and the phage clones that bind to Acenitobacter spp., in each round are retained. Optionally, the clones that bind to Acenitobacter spp., in each round are purified further by contacting the clones with one or more of Pasteurella pneumotropica and Staphylococcus aureus, and retaining those clones that do not bind to one or more of Pasteurella pneumotropica and Staphylococcus aureus. Optionally, the phage lysate obtained between each round of screening is subjected to precipitation e.g., using polyethylene glycol (PEG). Optionally, a sequence of a peptide expressed by a clone that binds reproducibly to Acenitobacter spp., is determined. Optionally, a synthetic peptide is produced that comprises a sequence of a peptide expressed by a clone that binds reproducibly to Acenitobacter spp., or a variant thereof e.g., comprising D-amino acids and/or a C-terminal amidated residue or a retro inverted analog of the peptide. Optionally, the minimum inhibitory concentration (MIC) of the synthetic peptide against Acenitobacter spp. is determined, and a peptide having a MIC value of about 25 μM or less than about 25 μM against Acenitobacter spp. is retained.

In the preceding examples, the peptide may be displayed on the surface of a cell or a phage. In accordance with this example of the invention, the method optionally additionally comprises determining the ability of an isolated form of the peptide, i.e., when not displayed on the surface of a cell or phage to bind to at least the first microorganism and/or to reduce or prevent growth of at least the first microorganism or to kill at least the first microorganism. In one example, the method comprises determining the ability of a plurality of peptides to bind to at least the first microorganism and/or to reduce or prevent growth of at least the first microorganism or to kill at least the first microorganism and separating a peptide that binds to at least the first microorganism and/or reduces or prevents growth of at least the first microorganism or kills at least the first microorganism from said plurality or library. Preferably, the term “separating” comprises the use of any chemical or biochemical purification process known in the art to fractionate the mixture of plurality of peptides coupled with assaying the fractions produced for an ability to reduce or prevent growth of a microorganism and/or kill a microorganism, and selecting fractions having an ability to reduce or prevent growth of a microorganism and/or kill a microorganism. More preferably, the term “separating” refers to a process comprises iterated use of any chemical or biochemical purification process known in the art to partially or completely purify a peptide or analog or derivative from a mixture of a plurality of peptides and assaying the fractions produced in each iteration of the process for an ability to reduce or prevent growth of a microorganism and/or kill a microorganism, and selecting at each iteration one or more fractions having an ability to reduce or prevent growth of a microorganism and/or kill a microorganism. Preferably, the process is repeated for n iterations wherein n is sufficient number of iterations to reach a desired purity of the compound e.g., 50% or 60% or 70% or 80% or 90% or 95% or 99%. More preferably, the process is repeated for zero to about ten iterations. As will be known to the skilled artisan, such iterations do not require iteration of precisely the same purification processes and more generally utilize different processes or purification conditions for each iteration. In the case of a library of peptides displayed separately wherein each peptide or analog or derivative is substantially pure prior to performance of the method, such isolation results in the separation of the peptide or analog or derivative from other compounds in the library that do not have the requisite activity. In this case, the term “separating” extends to determining the activity of one library component relative to another library component and selecting a peptide or analog or derivative having the desired activity.

In a further example, the peptide isolation method may additionally comprise determining the amino acid sequence or secondary or tertiary structure of a plurality of peptides capable of binding to at least the first microorganism and/or capable of reducing or preventing growth of at least the first microorganism or killing at least the first microorganism, and identifying one or more conserved sequences and/or secondary structures and/or tertiary structures in said plurality of peptides. Methods for determining the secondary or tertiary structure of a peptide or a fragment thereof will be apparent to the skilled artisan and include X-ray crystallography or nuclear magnetic resonance. Alternatively, or in addition, the secondary or tertiary structure of a peptide or a fragment thereof is predicted using an in silico method, e.g., threading.

In a further example, the peptide isolation method may additionally comprise producing one or more analog(s) or derivative(s) of the peptide isolated at (iv), determining the ability of the analog(s) or derivative(s) to bind to at least the first microorganism and/or to reduce or prevent growth of at least the first microorganism or to kill the first microorganism and isolating an analog or derivative that binds to at least the first microorganism and/or to reduces or prevents growth of at least the first microorganism or kills at least the first microorganism.

In a particular example, the present invention also provides a method for isolating an antimicrobial peptide or analog or derivative thereof, said method comprising:

(i) mutating or otherwise altering the amino acid sequence of a peptide or analog or derivative as described herein according to any embodiment to thereby produce a mutant peptide or mutant analog or mutant derivative;

(ii) determining the ability of the mutant peptide or mutant analog or mutant derivative to reduce or prevent growth of a microorganism and/or kill a microorganism; and

(iii) isolating a mutant peptide or mutant analog or mutant derivative that reduces or prevents growth of a microorganism and/or kills a microorganism.

Suitable methods for determining a mutant peptide or mutant analog or mutant derivative that reduces or prevents growth of a microorganism and/or kills a microorganism will be apparent to the skilled artisan and/or described herein. For example, the mutant peptide or mutant analog or mutant derivative is applied to a substrate upon which a microorganism has been previously grown or administered to a solution in which a microorganism has been previously grown and, after a suitable period of time, the level of growth inhibition and/or cell death of the microorganism is determined.

In one example, a mutant peptide or mutant analog or mutant derivative is isolated that has enhanced activity compared to the peptide or analog or derivative at (i). In another example, the method comprises determining the ability of a plurality of or a library of mutant peptides or mutant analogs or mutant derivatives to reduce or prevent growth of a microorganism and/or kill a microorganism, and separating a mutant peptide or mutant analog or mutant derivative that reduces or prevents growth of a microorganism and/or kills a microorganism from said plurality or library. In another example, the method additionally comprises providing or obtaining a mutant peptide or mutant analog or mutant derivative or a library of mutant peptides or a library of mutant analogs or a library of mutant derivatives.

The present invention also provides methods for isolating antibacterial compounds e.g., non-peptidyl compounds, such as small molecules. In on example. the present invention provides a method for isolating a compound that reduces or prevents growth of a microorganism and/or kills a microorganism, said method comprising:

(i) determining the structure of an antimicrobial peptide or analog or derivative according to any example hereof or a fusion protein fusion protein comprising said antimicrobial peptide or analog or derivative;

(ii) identifying, producing or obtaining one or more compounds that have a similar structure to the peptide, analog, derivative or fusion protein at (i) or that are predicted to have a similar structure to the peptide, analog, derivative or fusion protein at (i);

(iii) determining the ability of the one or more compounds at (ii) to reduce or prevent growth of a microorganism and/or kill a microorganism; and

(iv) isolating a compound that reduces or prevents growth of a microorganism and/or kills a microorganism.

It is to be understood that the present invention also extends to the direct product of this method e.g., a compound isolated by the method.

In another example, the method comprises:

(i) determining the structure of one or more peptides or analogs or derivatives as described herein according to any embodiment;

(ii) identifying, producing or obtaining one or more compounds, preferably non-peptidyl compounds, for example small molecule compounds that have a similar structure to the one or more peptides or analogs or derivatives or are predicted to have a similar structure to the one or more peptides or analogs or derivatives;

(iii) determining the ability of the compound or compounds to reduce or prevent growth of a microorganism and/or kill a microorganism; and

(iv) isolating a compound that reduces or prevents growth of a microorganism and/or kills a microorganism.

In another example, the method comprises determining the ability of a plurality of or a library of compounds to reduce or prevent growth of a microorganism and/or kill a microorganism and separating a compound that reduces or prevents growth of a microorganism and/or kills a microorganism from said plurality or library. The definition of the term “separating” and method steps recited in that definition described supra shall be taken to apply mutatis mutandis to the present embodiment of the invention.

In other examples, a method of isolating a peptide or analog or derivative or compound as described according to any example hereof additionally comprises:
(i) optionally, determining the structure of the peptide or analog or derivative or compound;
(ii) optionally, providing the name or structure of the peptide or analog or derivative or compound; and
(iii) providing the peptide or analog or derivative or compound.

In another example, the method additionally comprises providing or obtaining a compound or a library of compounds.

In another example, a method as described herein for isolating a peptide, analog, derivative or compound additionally comprises administering one or more peptides, analogs, derivative or compounds to a subject suffering from an infection caused by a microorganism; determining the effect of a peptide, analog, derivative or compound on the infection, and isolating a peptide that reduces the infection.

In another example, a method as described herein for isolating a peptide, analog, derivative or compound additionally comprises administering one or more peptides, analogs, derivative or compounds to a subject and subsequently contacting a subject with a microorganism for a time and under conditions for infection to occur in the absence of the peptide, analog, derivative or compound, and isolating a peptide that prevents infection of the subject by the microorganism.

In another example, the subject is a non-human animal, e.g., an animal model of an infection that occurs in humans.

The present invention clearly extends to the direct product of any method of identification or isolation of a peptide or analog or derivative or compound described herein according to any embodiment.

It is to be understood that an identified or isolated peptide or analog or derivative or compound in substantially pure form i.e., free from contaminants that might cause adverse side effects or contraindications or antagonize the activity of the active compound, can be formulated into a formulation to reduce or prevent growth of a microorganism and/or to kill a microorganism, e.g., to treat or prevent an infection or to sterilize or disinfect a composition of matter. Accordingly, in one example, the present invention additionally comprises formulating the isolated peptide or analog or derivative or compound with a suitable carrier or excipient.

### 3. General

This specification contains nucleotide and amino acid sequence information prepared using Patent In Version 3.3. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

### DETAILED DESCRIPTION OF THE PREFERRED EMBODIMENTS

Peptides

In a preferred embodiment, the present invention provides an antimicrobial peptide comprising at least seven or eight or ten or fifteen or twenty amino acids of any one or more of the sequences set forth in SEQ ID NOs: 1-167. Preferably, the peptide comprises at least about ten amino acids of any one or more of the sequences set forth in SEQ ID NOs: 1-167. More preferably, the peptide comprises at least fifteen amino acids of any one or more amino acid sequences set forth in SEQ ID NOs: 1-167. Still more preferably, the peptide comprises at least twenty amino acids of any one or more of the sequences set forth in SEQ ID NOs: 1-167.

Preferably, an antimicrobial peptide of the invention of analog thereof comprises an amino acid sequence at least about 70% identical to any one or more of the sequences set forth in SEQ ID NOs: 1-167. More preferably, the degree of sequence identity is at least about 75%. Even more preferably, the degree of sequence identity is at least about 80%. Still more preferably, the degree of sequence identity is at least about 85%. Even more preferably, the degree of sequence identity is at least about 90%. Still more preferably, the degree of sequence identity is at least about 95%. Still more preferably, the degree of sequence identity is at least about 99%, for example, 100%.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Madison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment.

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases and “blastp” used to align a known amino acid sequence with one or more sequences from one or more databases. Also available is a tool called “BLAST 2 Sequences” that is used for direct pairwise comparison of two nucleotide sequences.

As used herein the term “NCBI” shall be taken to mean the database of the National Center for Biotechnology Information at the National Library of Medicine at the National Institutes of Health of the Government of the United States of America, Bethesda, Md., 20894.

In this respect, non-natural amino acids shall be considered to be identical to their natural counterparts. Accordingly, a peptide comprising only non-natural amino acids (e.g., D-amino acids or N-methylated amino acids) equivalent to those set forth in SEQ ID NO: 1 shall be considered to have an amino acid sequence 100% identical to SEQ ID NO: 1.

Preferably, an antimicrobial peptide or derivative thereof or analog thereof is between about 6 to about 100 residues long (or any value there between), preferably from about 15 to 75 residues (or any value there between), preferably from about 20 to about 50 residues (or any value there between), and even more preferably from about 24 to about 40 residues (or any value there between).

Peptide Analogs

Suitable peptide analogs include, for example, a peptide comprising one or more conservative amino acid substitutions. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

It also is contemplated that other sterically similar compounds may be formulated to mimic the key portions of the peptide structure. Such compounds, which may be termed peptidomimetics, may be used in the same manner as an antimicrobial peptide of the invention. The generation of such an analog may be achieved by the techniques of modeling and chemical design known to those of skill in the art. It will be understood that all such sterically similar peptide analogs fall within the scope of the present invention.

Suitable peptide analogs include, for example, an antimicrobial peptide comprising one or more conservative amino acid substitutions. A “conservative amino acid substitution” is one in which the amino acid residue is replaced with an amino acid residue having a similar side chain.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), .beta.-branched side chains (e.g., threonine, valine, isoleucine) and aromatic side chains (e.g., tyrosine, phenylalanine, tryptophan, histidine).

Analogs of an antimicrobial of the invention include peptides in which one or more amino acids of the peptide structure are substituted with a homologous amino acid such that the properties of the original peptide are maintained, albeit not necessarily to the same level. For example, an analog can have enhanced or reduced activity to the native antimicrobial peptide from which it is derived and/or have a broader spectrum (e.g., having activity against a greater range of microbes) or be more specific. Preferably, conservative amino acid substitutions are made at one or more amino acid residues.

The importance of the hydropathic amino acid index in conferring interactive biological function on a protein is generally understood in the art (Kyte & Doolittle, J. Mol. Biol. 157, 105-132, 1982). It is known that certain amino acids may be substituted for other amino acids having a similar hydropathic index or score and still retain a similar biological activity, for example, the ability to bind to a membrane of a microorganism and/or to bind to a protein in or on a microorganism and/or kill a microorganism and/or reduce or prevent growth of a microorganism. The hydropathic index of amino acids also may be considered in determining a conservative substitution that produces a functionally equivalent molecule. Each amino acid has been assigned a hydropathic index on the basis of their hydrophobicity and charge characteristics, as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making changes based upon the hydropathic index, the substitution of amino acids whose hydropathic indices are within +/−0.2 is preferred. More preferably, the substitution will involve amino acids having hydropathic indices within +/−0.1, and more preferably within about +/−0.05.

It is also understood in the art that the substitution of like amino acids is made effectively on the basis of hydrophilicity. As detailed in U.S. Pat. No. 4,554,101, the following hydrophilicity values have been assigned to amino acid residues: arginine (+3.0); lysine (+3.0); aspartate (+3.0+/−0.1); glutamate (+3.0+/−0.1); serine (+0.3); asparagine (+0.2); glutamine (+0.2); glycine (0); threonine (−0.4); proline (−0.5+/−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, it is preferred to substitute amino acids having hydrophilicity values within about +/−0.2 of each other, more preferably within about +/−0.1, and even more preferably within about +/−0.05

The present invention also contemplates non-conservative amino acid changes. For example, of particular interest are substitutions of charged amino acids with another charged amino acid and with neutral or positively charged amino acids. The latter of these substitutions can produce an antimicrobial peptide analog having reduced positive charge, thereby improving the characteristics of the antimicrobial peptide.

It is also contemplated that other sterically similar compounds may be formulated to mimic the key portions of the peptide structure of an antimicrobial peptide of the invention. Such compounds, which may be termed peptidomimetics, may be used in the same manner as the peptides of the invention and hence are also analogs of a peptide of the invention. The generation of such an analog may be achieved by the techniques of modeling and chemical design known to those of skill in the art. It will be understood that all such sterically similar antimicrobial peptide analogs fall within the scope of the present invention.

Another method for determining the “equivalence” of modified peptides involves a functional approach. For example, a given peptide analog is tested for its antimicrobial activity e.g., using any screening method described herein and/or known in the art

An example of an analog of a peptide of the invention comprises one or more non-naturally occurring amino acids or amino acid analogs. For example, a peptide inhibitor as described herein comprises one or more naturally occurring non-genetically encoded L-amino acids, synthetic L-amino acids or D-enantiomers of an amino acid. For example, the peptide comprises only D-amino acids. In one example, the analog comprises one or more residues selected from the group consisting of: hydroxyproline, β-alanine, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylananine 3-benzothienyl alanine 4-chlorophenylalanine, 2-fluorophenylalanine, 3-fluorophenylalanine, 4-fluorophenylalanine, penicillamine, 1,2,3,4-tetrahydro-tic isoquinoline-3-carboxylic acid β-2-thienylalanine, methionine sulfoxide, homoarginine, N-acetyl lysine, 2,4-diamino butyric acid, ρ-aminophenylalanine, N-methylvaline, homocysteine, homoserine, ε-amino hexanoic acid, δ-amino valeric acid, 2,3-diaminobutyric acid and mixtures thereof.

Other amino acid residues that are useful for making the peptides and peptide analogs described herein can be found, e.g., in Fasman, 1989, CRC Practical Handbook of Biochemistry and Molecular Biology, CRC Press, Inc., and the references cited therein.

The present invention additionally encompasses an isostere of a peptide described herein. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets.

Other modifications include, for example, an N-alkyl (or aryl) substitution (ψ[CONR]), or backbone crosslinking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

In another example, a peptide analog is a retro-peptide analog (see, for example, Goodman et al., Accounts of Chemical Research, 12:1-7, 1979). A retro-peptide analog comprises a reversed amino acid sequence of a peptide inhibitor described herein. For example, a retro-peptide analog of a peptide inhibitor comprises a reversed amino acid sequence of a sequence set forth in any one of SEQ ID NOs: 1-140. Optionally, the peptide analog comprises an additional feature, such as, for example, a protein transduction domain, which may also be a retro-peptide.

In a further example, an analog of a peptide described herein is a retro-inverso peptide (as described, for example, in Sela and Zisman, FASEB J. 11:449, 1997). Evolution has ensured the almost exclusive occurrence of L-amino acids in naturally occurring proteins. As a consequence, virtually all proteases cleave peptide bonds between adjacent L-amino acids. Accordingly, artificial proteins or peptides composed of D-amino acids are preferably resistant to proteolytic breakdown. Retro-inverso peptide analogs are isomers of linear peptides in which the direction of the amino acid sequence is reversed (retro) and the chirality, D- or L-, of one or more amino acids therein is inverted (inverso) e.g., using D-amino acids rather than L-amino acids, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. An advantage of retro-inverso peptides is their enhanced activity in vivo due to improved resistance to proteolytic degradation, i.e., the peptide has enhanced stability. (e.g., Chorev et al., Trends Biotech. 13, 438-445, 1995).

Retro-inverso peptide analogs may be complete or partial. Complete retro-inverso peptides are those in which a complete sequence of a peptide described herein is reversed and the chirality of each amino acid in a sequence is inverted, other than glycine, because glycine does not have a chiral analog. Partial retro-inverso peptide analogs are those in which only some of the peptide bonds are reversed and the chirality of only those amino acid residues in the reversed portion is inverted. For example, two or more C-terminal and/or two or more N-terminal amino acids of a peptide are reversed in sequence and D-amino acids. For example, one or two or three or four or five or six or seven or eight or nine or ten or eleven or twelve or thirteen or fourteen or fifteen or sixteen or seventeen or eighteen or nineteen or twenty or twenty one or twenty two or twenty three or twenty four or twenty five or twenty six or twenty seven or twenty eight or twenty nine or thirty or thirty one or thirty two or thirty three or thirty four or thirty five or thirty six or thirty seven or thirty eight amino acid residues are D-amino acids. The present invention clearly encompasses both partial and complete retro-inverso peptide analogs. For example, the present invention provides a retroinverso analog of a peptide comprising a sequence set forth in any one or more of SEQ ID NOs: 1-167, in the sequence of any two or more amino acids is reversed and the reversed amino acids are D-amino acids. In one example, the present invention provides a complete retroinverso analog of a peptide comprising a sequence set forth in any one or more of SEQ ID NOs: 1-167 in which the entire sequence is reversed and all amino acids other than glycine are D-amino acids. In this respect, such a retroinverso peptide analog may optionally include an additional component, such as, for example, a pheromone or a protein transduction domain, which may also be retro inverted.

As will be apparent to the skilled artisan based on the foregoing description, the present invention contemplates an antimoicrobial peptide selected individually or collectively from the group consisting of:
(i) a peptide comprising an amino acid sequence set forth in any one of SEQ ID NOs: 1-167;
(ii) an antimicrobial peptide that is a variant of (i) having at least about 70% or 80% or 90% or 95% sequence identity thereto and comprising a sequence that differs from a sequence set forth in (i) or (ii) by one or more conservative amino acid substitutions, said variant being capable of reducing or preventing growth of one or more microorganisms and/or killing one or more microorganisms;
(iii) a peptide comprising a fragment of the sequence of (i) or (ii) said fragment being capable of reducing or preventing growth of one or more microorganisms and/or killing one or more microorganisms;
(iv) the peptide of any one of (i)-(iii) additionally comprising a pheromone;
(v) an analog of any one of (i) to (iv) selected from the group consisting of (a) the sequence of any one of (i) to (iv) comprising one or more non-naturally-occurring amino acids; (b) the sequence of any one of (i) to (iv) comprising one or more non-naturally-occurring amino acid analogs; (c) an isostere of any one of (i) to (iv); (d) a retro-peptide analog of any one of (i) to (iv); and (e) a retro-inverted peptide analog of any one of (i) to (iv).

The present invention also provides an antimicrobial peptide or analog thereof or derivative thereof selected individually or collectively from the group consisting of:
(i) a functional fragment of a peptide comprising an amino acid sequence set forth in any on of SEQ ID NOs: 1-167;
(ii) the peptide of (i) additionally comprising a pheromone; and
(iii) an analog of (i) or (ii) selected from the group consisting of (a) the sequence of (i) or (ii) comprising one or more non-naturally-occurring amino acids; (b) the sequence of (i) or (ii) comprising one or more non-naturally-occurring amino acid analogs; (c) an isostere of (i) or (ii); (d) a retro-peptide analog of (i) or (ii); and (e) a retro-inverted peptide analog of (i) or (ii).

As used herein the term “functional fragment” shall be taken to mean a fragment of a peptide or analog thereof that is capable of reducing or preventing growth of one or more microbes and/or killing one or more microbes. In this respect, the activity of a functional fragment need not be the same as that of the peptide or analog from which the fragment is derived. For example, the fragment may have enhanced or reduced activity compared to the peptide or analog from which it is derived.

In another embodiment, an analog of a peptide is modified to reduce the immunogenicity of said analog. Such reduced immunogenicity is useful for a peptide that is to be injected into a subject. Methods for reducing the immunogenicity of a peptide will be apparent to the skilled artisan. For example, an antigenic region of a peptide is predicted using a method known in the art and described, for example, in Kolaskar and Tongaonkar FEBS Letters, 276: 172-174, 1990. Any identified antigenic region may then be modified to reduce the immunogenicity of a peptide analog, provided that said analog is an antimicrobial peptide analog.

Alternatively, or in addition, Tangri et al., The Journal of Immunology, 174: 3187-3196, 2005, describe a process for identifying an antigenic site in a peptide and modifying said site to thereby reduce the immunogenicity of the protein without significantly reducing the activity of said protein. The approach is based on 1) the identification of immune-dominant epitopes, e.g., by determining binding to purified HLA molecules; and 2) reducing their binding affinity to HLA-DR molecules to levels below those associated with naturally occurring helper T lymphocyte epitopes. Generally, the approach is based on quantitative determination of HLA-DR binding affinity coupled with confirmation of these epitopes by in vitro immunogenicity testing.

Peptide Derivatives

In another example, the present invention provides a peptide derivative.

Peptide derivatives of the present invention encompass an antimicrobial peptide or an analog thereof as described herein in any embodiment that is modified to contain one or more-chemical moieties other than an amino acid. The chemical moiety may be linked covalently to the peptide or analog e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound (e.g., its antimicrobial activity).

An “amino terminal capping group” of a peptide compound described herein is any chemical compound or moiety that is covalently linked or conjugated to the amino terminal amino acid residue of a peptide or analog. An amino-terminal capping group may be useful to inhibit or prevent intramolecular cyclization or intermolecular polymerization, to protect the amino terminus from an undesirable reaction with other molecules, or to provide a combination of these properties. A peptide compound of this invention that possesses an amino terminal capping group can possess other beneficial activities as compared with the uncapped peptide, such as enhanced efficacy or reduced side effects. Examples of amino terminal capping groups that are useful in preparing peptide derivatives according to the invention include, but are not limited to, 1 to 6 naturally occurring L-amino acid residues, preferably, 1-6 lysine residues, 1-6 arginine residues, or a combination of lysine and arginine residues; urethanes; urea compounds; lipoic acid (“Lip”); glucose-3-O-glycolic acid moiety (“Gga”); or an acyl group that is covalently linked to the amino terminal amino acid residue of a peptide, wherein such acyl groups useful in the compositions of the invention may have a carbonyl group and a hydrocarbon chain that ranges from one carbon atom (e.g., as in an acetyl moiety) to up to 25 carbons (e.g., palmitoyl group, “Palm” (16:0) and docosahexaenoyl group, “DHA” (C22:6-3)). Furthermore, the carbon chain of the acyl group may be saturated, as in Palm, or unsaturated, as in DHA. It is understood that when an acid, such as docosahexaenoic acid, palmitic acid, or lipoic acid is designated as an amino terminal capping group, the resultant peptide compound is the condensed product of the uncapped peptide and the acid.

A “carboxy terminal capping group” of a peptide compound described herein is any chemical compound or moiety that is covalently linked or conjugated to the carboxy terminal amino acid residue of the peptide compound. The primary purpose of such a carboxy terminal capping group is to inhibit or prevent intramolecular cyclization or intermolecular polymerization, to promote transport of the peptide compound across a cell membrane, or to provide a combination of these properties. A peptide of this invention possessing a carboxy terminal capping group may also possess other beneficial activities as compared with the uncapped peptide, such as enhanced efficacy, reduced side effects, enhanced hydrophilicity, and enhanced hydrophobicity. Carboxy terminal capping groups that are particularly useful in the peptide compounds described herein include primary or secondary amines that are linked by an amide bond to the α-carboxyl group of the carboxy terminal amino acid of the peptide. Other carboxy terminal capping groups useful in the invention include aliphatic primary and secondary alcohols and aromatic phenolic derivatives, including flavenoids, with 1 to 26 carbon atoms,' which form esters when linked to the carboxylic acid group of the carboxy terminal amino acid residue of a peptide described herein.

Other chemical modifications of a peptide or analog, include, for example, glycosylation, acetylation (including N-terminal acetylation), carboxylation, carbonylation, phosphorylation, PEGylation, amidation, addition of trans olefin, substitution of α-hydrogens with methyl groups, derivatization by known protecting/blocking groups, circularization, inhibition of proteolytic cleavage (e.g., using D amino acids), linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, etc.

Fusion Proteins and Complexes

1. Protein Transduction Domains

In the case of an antimicrobial peptide that acts within a microorganism, e.g., a fungal cell or a protozoal cell, to facilitate peptide entry into a cell, the peptide can be conjugated to (e.g., expressed as a fusion with) a protein transduction domain or an analog or derivative thereof. As used herein, the term “protein transduction domain” shall be taken to mean a peptide or protein that is capable of enhancing, increasing or assisting penetration or uptake of a compound conjugated to the protein transduction domain into a cell either in vitro or in vivo. Those skilled in the art will be aware that synthetic or recombinant peptides can be delivered into cells through association with a protein transduction domain such as the TAT sequence from HIV or the Penetratin sequence derived from the Antennapaedia homeodomain protein (see, for example, Temsamani and Vidal, Drug Discovery Today 9: 1012-1019, 2004, for review).

A suitable protein transduction domain will be apparent to the skilled artisan and includes, for example, a cC10 pheromone peptide e.g., comprising a sequnce set forth in SEQ ID NO: 168, an ABC transporter or permease (e.g., comprising an amino acid sequnce set forth in SEQ ID NO: 169), a basic region of HIV-1 TAT protein (e.g., comprising an amino acid sequence set forth in SEQ ID NO: 170), or transportan (e.g., comprising an amino acid sequence set forth in SEQ ID NO: 171).

Additional suitable protein transduction domains are described, for example, in Zhao and Weisledder Medicinal Research Reviews, 24: 1-12, 2004 and Wagstaff and Jans, Current Medicinal Chemistry, 13: 1371-1387, 2006.

2. Multimeric Proteins

In another embodiment, a fusion protein of the present invention comprises a plurality of antimicrobial peptides of the invention and/or analogs thereof. In this respect, the fusion protein may comprise multiple copies of the same antimicrobial peptide or analog and/or a plurality of antimicrobial peptides and/or analogs (whether present in a single copy or a plurality of copies).

In one embodiment, such a fusion protein comprises one or more additional components, such as, for example, a tag or label and/or an additional antimicrobial peptide or analog or derivative thereof.

3. Linkers

Each of the components of a derivative of an antimicrobial peptide of the invention may optionally be separated by a linker that facilitates the independent folding of each of said components. A suitable linker will be apparent to the skilled artisan. For example, it is often unfavourable to have a linker sequence with high propensity to adopt α-helix or β-strand structures, which could limit the flexibility of the protein and consequently its functional activity. Rather, a more desirable linker is a sequence with a preference to adopt extended conformation. In practice, most currently designed linker sequences have a high content of glycine residues that force the linker to adopt loop conformation. Glycine is generally used in designed linkers because the absence of a β-carbon permits the polypeptide backbone to access dihedral angles that are energetically forbidden for other amino acids.

Preferably, the linker is hydrophilic, i.e. the residues in the linker are hydrophilic.

Linkers comprising glycine and/or serine have a high freedom degree for linking of two proteins, i.e., they enable the fused proteins to fold and produce functional proteins.

In one embodiment, the linker is a glycine rich linker. Preferably, the linker is a glycine linker optionally, additionally comprising alanine and/or serine.

Peptide Synthesis

An antimicrobial peptide of the invention or an analog or derivative thereof is preferably synthesized using a chemical method known to the skilled artisan. For example, synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Generally, chemical synthesis methods comprise the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol. 1, for classical solution synthesis. These methods are suitable for synthesis of an antimicrobial peptide of the present invention or an analog or derivative thereof.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The antimicrobial peptide, analog or derivative of the present invention can also be chemically prepared by other methods such as by the method of simultaneous multiple peptide synthesis. See, e. g., Houghten Proc. Natl. Acad. Sci. USA 82: 5131-5135, 1985 or U.S. Pat. No. 4,631,211.

As will be apparent to the skilled artisan based on the description herein, an analog or derivative of an antimicrobial of the invention may comprise D-amino acids, a combination of D- and L-amino acids, and various unnatural amino acids (e.g., α-methyl amino acids, Cα-methyl amino acids, and Nα-methyl amino acids, etc) to convey special properties. Synthetic amino acids include ornithine for lysine, fluorophenylalanine for phenylalanine, and norleucine for leucine or isoleucine. Methods for the synthesis of such peptides will be apparent tot eh skilled artisan based on the foregoing.

Recombinant Peptide Production

In one embodiment, an antimicrobial peptide or analog or derivative thereof or fusion protein comprising same is produced as a recombinant protein. To facilitate the production of a recombinant peptide or fusion protein nucleic acid encoding same is preferably isolated or synthesized. Typically, the nucleic acid encoding the constituent components of the fusion protein is/are isolated using a known method, such as, for example, amplification (e.g., using PCR or splice overlap extension) or isolated from nucleic acid from an organism using one or more restriction enzymes or isolated from a library of nucleic acids. Methods for such isolation will be apparent to the ordinary skilled artisan and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

For example, nucleic acid (e.g., genomic DNA or RNA that is then reverse transcribed to form cDNA) from a cell or organism capable of expressing an antimicrobial peptide of the invention is isolated using a method known in the art and cloned into a suitable vector. The vector is then introduced into a suitable organism, such as, for example, a bacterial cell. Using a nucleic acid probe from a known antimicrobial peptide encoding gene a cell comprising the nucleic acid of interest is isolated using methods known in the art and described, for example, in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

Alternatively, nucleic acid encoding an antimicrobial peptide of the invention is isolated using polymerase chain reaction (PCR). Methods of PCR are known in the art and described, for example, in Dieffenbach (ed) and Dveksler (ed) (In: PCR Primer: A Laboratory Manual, Cold Spring Harbour Laboratories, N.Y., 1995). Generally, for PCR two non-complementary nucleic acid primer molecules comprising at least about 20 nucleotides in length, and more preferably at least 25 nucleotides in length are hybridized to different strands of a nucleic acid template molecule, and specific nucleic acid molecule copies of the template are amplified enzymatically. Preferably, the primers hybridize to nucleic acid adjacent to a nucleic acid encoding an antimicrobial peptide of the invention, thereby facilitating amplification of the nucleic acid that encodes the subunit. Following amplification, the amplified nucleic acid is isolated using a method known in the art and, preferably cloned into a suitable vector.

Other methods for the production of a nucleic acid of the invention will be apparent to the skilled artisan and are encompassed by the present invention.

For expressing protein by recombinant means, a protein-encoding nucleotide sequence is placed in operable connection with a promoter or other regulatory sequence capable of regulating expression in a cell-free system or cellular system. For example, nucleic acid comprising a sequence that encodes an antimicrobial peptide of the present invention in operable connection with a suitable promoter is expressed in a suitable cell for a time and under conditions sufficient for expression to occur. Nucleic acid encoding an antimicrobial protein of the present invention is readily derived from the publicly available amino acid sequence.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid (e.g., a transgene), e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid (e.g., a transgene) to which it is operably linked. Preferred promoters can contain additional copies of one or more specific regulatory elements to further enhance expression and/or alter the spatial expression and/or temporal expression of said nucleic acid.

As used herein, the term “in operable connection with” “in connection with” or “operably linked to” means positioning a promoter relative to a nucleic acid (e.g., a transgene) such that expression of the nucleic acid is controlled by the promoter. For example, a promoter is generally positioned 5′ (upstream) to the nucleic acid, the expression of which it controls. To construct heterologous promoter/nucleic acid combinations (e.g., promoter/transgene combinations), it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function.

Should it be preferred that a peptide or fusion protein of the invention is expressed in vitro a suitable promoter includes, but is not limited to a T3 or a T7 bacteriophage promoter (Hanes and Plückthun Proc. Natl. Acad. Sci. USA, 94 4937-4942 1997).

Typical expression vectors for in vitro expression or cell-free expression have been described and include, but are not limited to the TNT T7 and TNT T3 systems (Promega), the pEXP1-DEST and pEXP2-DEST vectors (Invitrogen).

Typical promoters suitable for expression in bacterial cells include, but are not limited to, the lacz promoter, the Ipp promoter, temperature-sensitive λL or λR promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are well-known in the art and are described for example, in Ausubel et al (In: Current Protocols in Molecular Biology. Wiley Interscience, ISBN 047 150338, 1987), U.S. Pat. No. 5,763,239 (Diversa Corporation) and Sambrook et al (In: Molecular Cloning: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have been described, and include, for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO or pBAD/thio—TOPO series of vectors containing an arabinose-inducible promoter (Invitrogen, Carlsbad, Calif.), the latter of which is designed to also produce fusion proteins with a Trx loop for conformational constraint of the expressed protein; the pFLEX series of expression vectors (Pfizer Inc., CT, USA); the pQE series of expression vectors (QIAGEN, CA, USA), or the pL series of expression vectors (Invitrogen), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (e.g., 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991).

A wide range of additional host/vector systems suitable for expressing an antimicrobial peptide or fusion protein of the present invention are available publicly, and described, for example, in Sambrook et al (In: Molecular cloning, A laboratory manual, second edition, Cold Spring Harbor Laboratory, Cold Spring Harbor, N.Y., 1989).

Means for introducing an isolated nucleic acid or a gene construct comprising same or expression vector into a cell for expression are known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

Peptide/Analog/Derivative/Fusion Protein Isolation

Following production/expression/synthesis, an antimicrobial peptide of the invention or derivative or analog thereof or fusion protein comprising same is purified using a method known in the art. Such purification preferably provides a peptide of the invention substantially free of conspecific protein, acids, lipids, carbohydrates, and the like. Antibodies and other affinity ligands are particularly preferred for producing isolated protein. Preferably, the protein will be in a preparation wherein more than about 90% (e.g. 95%, 98% or 99%) of the protein in the preparation is an antimicrobial peptide of the invention or derivative or analog thereof or fusion protein comprising same.

Standard methods of peptide purification are employed to obtain an isolated peptide of the invention, including but not limited to various high-pressure (or performance) liquid chromatography (HPLC) and non-HPLC peptide isolation protocols, such as size exclusion chromatography, ion exchange chromatography, phase separation methods, electrophoretic separations, precipitation methods, salting in/out methods, immunochromatography, and/or other methods.

A preferred method of isolating peptide compounds useful in compositions and methods of the invention employs reversed-phase HPLC using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can also be used to separate a peptide based on its charge.

Alternatively, affinity purification is useful for isolating a fusion protein comprising a label. Methods for isolating a protein using affinity chromatography are known in the art and described, for example, in Scopes (In: Protein purification: principles and practice, Third Edition, Springer Verlag, 1994). For example, an antibody or compound that binds to the label (in the case of a polyhistidine tag this may be, for example, nickel-NTA) is preferably immobilized on a solid support. A sample comprising a fusion protein is then contacted to the immobilized antibody or compound for a time and under conditions sufficient for binding to occur. Following washing to remove any unbound or non-specifically bound protein, the fusion protein is eluted.

The degree of purity of the peptide compound may be determined by various methods, including identification of a major large peak on HPLC. A peptide compound that produces a single peak that is at least 95% of the input material on an HPLC column is preferred. Even more preferable is a polypeptide that produces a single peak that is at least 97%, at least 98%, at least 99% or even 99.5% of the input material on an HPLC column.

To ensure that a peptide obtained using any of the techniques described above is the desired peptide for use in compositions and methods of the present invention, analysis of the composition of the peptide is determined by any of a variety of analytical methods known in the art. Such composition analysis may be conducted using high resolution mass spectrometry to determine the molecular weight of the peptide. Alternatively, the amino acid content of a peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine the sequence of the peptide. Since some of the peptide compounds contain amino and/or carboxy terminal capping groups, it may be necessary to remove the capping group or the capped amino acid residue prior to a sequence analysis. Thin-layer chromatographic methods may also be used to authenticate one or more constituent groups or residues of a desired peptide.

Producing Modified Forms of Antimicrobial Peptides

Methods for producing modified forms an antimicrobial peptide of the invention will be apparent to the skilled artisan and include recombinant methods. For example, a nucleic acid encoding an antimicrobial peptide of the invention or an analog thereof is amplified using mutagenic PCR and the resulting nucleic acid expressed to produce a peptide using a method known in the art and/or described herein.

In a preferred embodiment, the nucleic acid fragments are modified by amplifying a nucleic acid fragment using mutagenic PCR. Such methods include a process selected from the group consisting of: (i) performing the PCR reaction in the presence of manganese; and (ii) performing the PCR in the presence of a concentration of dNTPs sufficient to result in mis-incorporation of nucleotides.

Methods of inducing random mutations using PCR are known in the art and are described, for example, in Dieffenbach (ed) and Dveksler (ed) (In: PCR Primer: A Laboratory Manual, Cold Spring Harbour Laboratories, NY, 1995). Furthermore, commercially available kits for use in mutagenic PCR are obtainable, such as, for example, the Diversify PCR Random Mutagenesis Kit (Clontech) or the GeneMorph Random Mutagenesis Kit (Stratagene).

In one embodiment, PCR reactions are performed in the presence of at least about 200 μM manganese or a salt thereof, more preferably at least about 300 μM manganese or a salt thereof, or even more preferably at least about 500 μM or at least about 600 μM manganese or a salt thereof. Such concentrations manganese ion or a manganese salt induce from about 2 mutations per 1000 base pairs (bp) to about 10 mutations every 1000 by of amplified nucleic acid (Leung et al Technique 1, 11-15, 1989).

In another embodiment, PCR reactions are performed in the presence of an elevated or increased or high concentration of dGTP. It is preferred that the concentration of dGTP is at least about 25 μM, or more preferably between about 50 μM and about 100 μM. Even more preferably the concentration of dGTP is between about 100 μM and about 150 μM, and still more preferably between about 150 μM and about 200 μM. Such high concentrations of dGTP result in the mis-incorporation of nucleotides into PCR products at a rate of between about 1 nucleotide and about 3 nucleotides every 1000 by of amplified nucleic acid (Shafkhani et al BioTechniques 23, 304-306, 1997).

PCR-based mutagenesis is preferred for the mutation of the nucleic acid fragments of the present invention, as increased mutation rates are achieved by performing additional rounds of PCR.

Alternatively, or in addition, a nucleic acid encoding an antimicrobial peptide of the invention or a derivative thereof is inserted or introduced into a host cell that is capable of mutating nucleic acid. Such host cells are generally deficient in one or more enzymes, such as, for example, one or more recombination or DNA repair enzymes, thereby enhancing the rate of mutation to a rate that is rate approximately 5,000 to 10,000 times higher than for non-mutant cells. Strains particularly useful for the mutation of nucleic acids carry alleles that modify or inactivate components of the mismatch repair pathway. Examples of such alleles include alleles selected from the group consisting of mutY, mutM, mutD, mutT, mutA, mutC and mutS. Bacterial cells that carry alleles that modify or inactivate components of the mismatch repair pathway are known in the art, such as, for example the XL-1Red, XL-mutS and XL-mutS-Kanr bacterial cells (Stratagene).

Alternatively the nucleic acid is cloned into a nucleic acid vector that is preferentially replicated in a bacterial cell by the repair polymerase, Pol I. By way of exemplification, a Pol I variant strain will induce a high level of mutations in the introduced nucleic acid vector, thereby enhancing sequence diversity of the nucleic acid encoding the antimicrobial peptide or derivative thereof. Such a method is described, for example, in Fabret et al (In: Nucl Acid Res, 28: 1-5 2000).

Alternatively, derivatives of an antimicrobial peptide of the present invention can be generated through DNA shuffling, e.g., as disclosed in Stemmer, Nature 370:389-91, 1994, Stemmer, Proc. Natl. Acad. Sci. USA 91:10747-51, 1994 and WO 97/20078. Briefly, nucleic acid encoding a derivative of the invention is generated by in vitro homologous recombination by random fragmentation of a parent DNA (e.g., encoding an antimicrobial peptide of the invention) followed by reassembly using PCR, resulting in randomly introduced mutations. This technique can be modified by using a family of parent DNAs, such as, for example, nucleic acid encoding another antimicrobial peptide, to introduce additional variability into the process. Reassembled nucleic acids are then expressed to produce a derivative peptide and assessed for antimicrobial activity and/or reduced immunogenicity and/or resistance to degradation using a method known in the art and/or described herein. Screening for the desired activity, followed by additional iterations of mutagenesis and assay provides for rapid “evolution” of sequences by selecting for desirable mutations while simultaneously selecting against detrimental changes.

For example, a derivative of the invention is produced by combining nucleic acids encoding two or more antimicrobial peptides of the invention, or nucleic acid encoding one or more antimicrobial peptides of the invention and nucleic acid encoding another antimicrobial peptide in a reaction vessel. The nucleic acids are then digested using a nuclease (e.g., DNase I). The resulting fragments are then reassembled by repeated cycles of denaturing and annealing in the presence of a DNA polymerase. Homologous regions of fragments then induce DNA replication of fragments, e.g., from different source templates, to thereby regenerate a nucleic acid encoding a peptide analog. Such a method is described, for example, in Stemmer, Proc. Natl. Acad. Sci. USA 91:10747-51, 1994. An analog produced using this method may then be screened for antimicrobial activity, e.g., using a method described herein.

The present invention additionally encompasses the production of a modified form of an antimicrobial peptide of the invention by performing a combination of random mutagenesis and DNA shuffling.

Alternatively, a modified form of an antimicrobial peptide of the invention is produced by performing site-directed mutagenesis. Suitable methods of site-directed mutagenesis are known in the art and/or described in Dieffenbach (ed) and Dveksler (ed) (In: PCR Primer: A Laboratory Manual, Cold Spring Harbour Laboratories, NY, 1995).

Alternatively, a modified form of an antimicrobial peptide of the present invention is produced by systematically substituting amino acids in the sequence of one or more antimicrobial peptides using synthetic means.

Determining the Antimicrobial Activity of a Peptide

Methods for determining the antimicrobial activity of a peptide will be apparent to the skilled artisan, for example, based on the description herein.

For example, antimicrobial activity of a peptide is determined using a broth dilution method. Essentially, this method involves growing a microorganism in liquid media until log phase is reached. The peptide, analog or derivative to be tested is serially diluted in media in which the microorganism is grown and a sample of the microorganism added to the peptide containing sample. The sample is then maintained for a time and under conditions sufficient for growth of the microorganism, and the amount of growth of the microorganism determined relative to a negative control by detecting the absorbance, e.g., at A₆₀₀.

Another method for determining the antimicrobial activity of a peptide of the invention or an analog or derivative thereof is a redial diffusion assay. For example, a lawn of a microorganism is grown and a specific location within that lawn is contacted with a sample of the peptide, analog or derivative, and the area surrounding the site of contacting the peptide is determined. A peptide that inhibits growth of the microorganism in and/or around the site of contact is considered an antimicrobial peptide. The range or diameter of growth inhibition is considered to be related to the antimicrobial activity of the peptide.

Another method in accordance with the invention comprises contacting a microorganism previously contacted with a peptide to be tested with an agent that has affinity for a compound located within the microorganism, but is not able to cross an intact or undamaged membrane. The presence of the agent within the microorganism indicates that the agent crossed the membrane indicating that the membrane of the microorganism was damaged by the peptide. An example of such an agent is Sytox green dye (Molecular Probes, Eugene, Oreg.). This dye has a strong affinity for nucleic acids, but can only penetrate cells that have a damaged membrane.

Yet another method for determining whether a peptide being assayed for antimicrobial activity has damaged the membrane of the microorganism involves contacting the microorganism with a test peptide and an agent capable of crossing the membrane of the microorganism. The agent is capable of being processed within the microorganism to form a product that is unable to cross an undamaged membrane. The medium surrounding the microorganism is then assayed for the presence of said product. The presence of said product in the medium in which the microorganism is grown is indicative of damage to the membrane of the microorganism caused by the peptide, and is indicative of the antimicrobial activity of the peptide. An example of a suitable agent is calcein AM. Calcein AM is converted into free calcein within the microorganism. Normally, free calcein is unable to cross the cell membrane of the microorganism and enter the surrounding culture. Thus, detection of free calcein in the medium surrounding the microorganism is indicative of damage to the cell membrane of the microorganism, and thus the antimicrobial activity of the peptide.

A further method for determining antimicrobial activity of a peptide, preferably of a plurality of peptides is described, for example, in Hilpert et al, Nature Biotechnology, 23: 1008-1012, 2005. Briefly, this method comprises robotically spotting peptides or synthesizing peptides on a cellulose sheet, and assaying the ability of each peptide when released from the cellulose sheet to decrease ATP-dependent luminescence in a luciferase-expressing reporter strain of a microorganism, e.g., P. aeruginosa.

Alternatively, or in addition, an antimicrobial peptide of the invention or analog or derivative thereof is administered to an animal model of infection and the effect of the peptide on said infection is determined. Animal models of infection are known in the art and include, for example, primate models of HIV-1 infection (Nathanson Int J STD AIDS; 9 1:3-7, 1989); rat, mouse or monkey models of candidiasis (Samaranayake and Samaranayake Clinical Microbiology Reviews, 398-429, 2001); mouse models of S. aureus infection (Kuklin et al., Antimicrobial Agents and Chemotherapy 47: 2740-2748, 2003); a mouse model of chronic P. aeruginosa infection (van Heeckeren, Lab Anim. 36: 291-312, 2002, and/or an animal model described in Bacterial Pathogenesis, Part A: Identification And Regulation Of Virulence Factors, 235 (Clark et al., Eds.), Academic Press, 1994. A mouse model of A. baumannii mediated pneumonia is described in Rodríguez-Hernández et al., Journal of Antimicrobial Chemotherapy 45: 493-501, 2000. A rabbit model of A. baumannii-mediated endocarditis is described in Perlman & Freedman, Yale Journal of Biology and Medicine 44: 206-13, 1971.

Pharmaceutical Formulations Comprising an Antimicrobial Peptide, Analog or Derivative

A peptide or analog thereof or derivative thereof of the present invention may be administered alone but will preferably be administered as a pharmaceutical composition, which will generally comprise a suitable pharmaceutically acceptable excipient, diluent or carrier selected depending on the intended route of administration. Examples of suitable pharmaceutical carriers include; water, glycerol, ethanol and other GRAS reagents.

The term “carrier or excipient” as used herein, refers to a carrier or excipient that is conventionally used in the art to facilitate the storage, administration, and/or the biological activity of an active compound. A carrier may also reduce any undesirable side effects of the active compound. A suitable carrier is, for example, stable, e.g., incapable of reacting with other ingredients in the formulation. In one example, the carrier does not produce significant local or systemic adverse effect in recipients at the dosages and concentrations employed for treatment. Such carriers and excipients are generally known in the art. Suitable carriers for this invention include those conventionally used, e.g., water, saline, aqueous dextrose, and glycols are preferred liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

The skilled artisan will be aware of a suitable carrier or excipient. For example, a carrier or excipient does not inhibit the activity of a peptide or analog or derivative of the invention in reducing or preventing growth of a microorganism and/or killing a microorganism.

The formulations can be subjected to conventional pharmaceutical expedients, such as sterilization, and can contain a conventional pharmaceutical additive, such as a preservative and/or a stabilizing agent and/or a wetting agent and/or an emulsifying agent and/or a salt for adjusting osmotic pressure and/or a buffer and/or other additive known in the art. Other acceptable components in the formulation of the invention include, but are not limited to, isotonicity-modifying agents such as water and/or saline and/or a buffer including phosphate, citrate, succinate, acetic acid, or other organic acids or their salts.

In an example, a formulation includes one or more stabilizers, reducing agents, anti-oxidants and/or anti-oxidant chelating agents. The use of buffers, stabilizers, reducing agents, anti-oxidants and chelating agents in the preparation of compositions, is known in the art and described, for example, in Wang et al. J. Parent. Drug Assn. 34:452-462, 1980; Wang et al. J. Parent. Sci. Tech. 42:S4-S26 (Supplement), 1988. Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

In another example, a formulation comprises a peptidyl moiety conjugated to a hydrolysable polyethylene glycol (PEG) essentially as described by Tsubery et al., J. Biol. Chem. 279 (37) pp. 38118-38124. Without being bound by any theory or mode of action, such formulations provide for extended or longer half-life of the peptide moiety in circulation. For example, to increase the serum half life of a peptide or analog or derivative of the present invention it may be so conjugated either to higher molecular weight compounds such as PEG which have slower renal clearance. Examples of hydrolysable PEG linkers have been described (eg. Hong Zhao et al., Bioconjugate Chem.' 17: 341-351, 206; or in Tsubery et al., Journal of Biological Chemistry 279: 38118-38124, 2004. Alternatively, they may be released more slowly from a nanoparticle such as hydrogel, PLGA. Another method to extend serum half-life involves joining of the peptide sequence to a sequence which has the capacity to bind to an abundant serum protein such as human serum albumin.

In another example, a formulation comprises a nanoparticle comprising the peptide moiety or other active ingredient bound to it or encapsulated within it. Without being bound by any theory or mode of action, delivery of a peptidyl composition from a nanoparticle may reduce renal clearance of the peptide(s).

As will be apparent to the skilled artisan, the ultimate form that a formulation may take will vary according to the route of administration selected (e.g., solution, emulsion, capsule). Pharmaceutical formulations can be adapted for administration by any appropriate route, for example by oral (including buccal or sublingual), rectal, nasal, topical (including buccal, sublingual or transferal), vaginal or parenteral (including subcutaneous, intramuscular, intravenous or intradermal) route. Such formulations can be prepared by any method known in the art of pharmacy, for example by bringing into association the active ingredient with the carrier(s), diluent(s) or excipient(s).

To prepare such pharmaceutical formulations, one or more peptides or analogs or nucleic acids or cells of the present invention is/are mixed with a pharmaceutically acceptable carrier or excipient for example, by mixing with physiologically acceptable carriers, excipients, or stabilizers in the form of, e.g., lyophilized powders, slurries, aqueous solutions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Preferably, a formulation of the invention also includes one or more stabilizers, reducing agents, anti-oxidants and/or anti-oxidant chelating agents. The use of buffers, stabilizers, reducing agents, anti-oxidants and chelating agents in the preparation of protein-based compositions, is known in the art and described, for example, in Wang et al. J. Parent. Drug Assn. 34:452-462, 1980; Wang et al. J. Parent. Sci. Tech. 42:S4-S26 (Supplement), 1988. Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

Pharmaceutical formulations may be presented in unit dose forms containing a predetermined amount of active ingredient per unit dose.

A discussion of preferred forms of formulations depending on the route of administration follows:

a) Oral Formulations

Pharmaceutical formulations adapted for oral administration may be presented as discrete units such as capsules, soft gels, or tablets; powders or granules; solutions or suspensions in aqueous or non-aqueous liquids; edible foams or whips; or oil-in-water liquid emulsions or water-in-oil liquid emulsions. Alternatively, an oral formulation is a mouthwash or gargle or other liquid for treating an infection in the mouth or throat of a subject without requiring the subject to actually swallow or ingest the formulation. Such formulations are suitable for the treatment of a variety of infections, including an infection of the mouth or throat or digestive system or an infection treated by system administration of a peptide/analog/derivative of the present invention.

Granular Tablets and Capsules

In one example, the oral formulation comprises an intragranular phase comprising an effective amount of a peptide or analog of the present invention and at least one carbohydrate alcohol and an aqueous binder. Preferably, the pharmaceutical formulation is substantially lactose-free. Preferred carbohydrate alcohols for such formulations are selected from the group consisting of mannitol, maltitol, sorbitol, lactitol, erythritol and xylitol. Preferably, the carbohydrate alcohol is present at a concentration of about 15% to about 90%. A preferred aqueous binder is selected from the group consisting of hydroxypropyl cellulose, hydroxypropyl methylcellulose, carboxymethyl cellulose sodium, polyvinyl pyrrolidones, starches, gelatins and povidones. A binder is generally present in the range of from about 1% to about 15%. The intragranular phase can also comprise one or more diluents, such as, for example, a diluent selected from the group consisting of microcrystalline cellulose, powdered cellulose, calcium phosphate-dibasic, calcium sulfate, dextrates, dextrins, alginates and dextrose excipients. Such diluents are also present in the range of about 15% to about 90%. The intragranular phase can also comprise one or more disintegrants, such as, for example, a disintegrant selected from the group consisting of a low substituted hydroxypropyl cellulose, carboxymethyl cellulose, calcium carboxymethylcellulose, sodium carboxymethyl cellulose, sodium starch glycollate, crospovidone, croscarmellose sodium, starch, crystalline cellulose, hydroxypropyl starch, and partially pregelatinized starch. A disintegrant is generally present in the range of from about 5% to about 20%. Such a formulation can also comprise one or more lubricants such as, for example, a lubricant selected from the group consisting of talc, magnesium stearate, stearic acid, hydrogenated vegetable oils, glyceryl behenate, polyethylene glycols and derivatives thereof. A lubricant is generally present in the range of from about 0.5% to about 5%.

Hard or Soft Gels

A liquid or semi-solid pharmaceutical formulation for oral administration e.g., a hard gel or soft gel capsule, may be prepared comprising:

(a) a first carrier component comprising from about 10% to about 99.99% by weight of a peptide or analog of the present invention;

(b) an optional second carrier component comprising, when present, up to about 70% by weight of said peptide or analog;

(c) an optional emulsifying/solubilizing component comprising, when present, from about 0.01% to about 30% by weight of said peptide or analog;

(d) an optional anti-crystallization/solubilizing component comprising, when present, from about 0.01% to about 30% by weight of said peptide or analog; and

(e) an active pharmacological agent comprising from about 0.01% to about 80% of said peptide or analog.

The first carrier component and optional second carrier component generally comprise, independently, one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil.

The emulsifying/solubilizing component generally comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

The anti-crystallization/solubilizing component, when present, generally comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

b) Topical Formulations and Patches and Wound Dressings

Topical formulations comprising a peptide and/or analog and/or derivative of the present invention are also useful for treating a variety of disorders, e.g., by local of systemic administration. For example, a topical formulation is useful fro treating an infection of the skin and/or for preventing such an infection e.g., in a wound.

Pharmaceutical formulations adapted for transferal administration may be presented as discrete patches intended to remain in intimate contact with the epidermis of the recipient or to cover a wound in the skin of a subject for a prolonged period of time. For example, the active ingredient may be delivered from the patch by iontophoresis as generally described in Pharmaceutical Research, 3(6), p 318 et seq. (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas; rectal ointments and foams may also be employed.

Pharmaceutical formulations adapted for vaginal administration may be presented as pessaries, tampons, creams, gels, pastes, foams or spray formulations.

The skilled artisan will be aware of suitable carriers for topical formulations. In one example, the carrier is a co-polymer. For example, Puolakkainen et al., J. Urg. Res., 58: 321-329 describes a variety of carriers suitable for topical formulations, e.g., describe a poly(ethylene oxide)-poly(propylene oxide) block copolymer designated Pluronic F-127. Pluronic F-127 has been used as a carrier for a variety of peptides and proteins in addition to nucleic acid based compounds. This carrier exhibits thermoreversability, relative inertness toward protein and nucleic acid and low toxicity.

In another example, the carrier is a paste. For example, DuoDERM is a hydroactive paste, comprising sodium carboxymethylcellulose, gelatin, pectin and polyisobutylene (Alvarez et al., J. Surg. Res., 35: 142, 1983).

In a further example, the carrier is a hydrogel. For example, biopol hydrogel is a poly(ethylene oxide) cross-linked hydrogel that interacts with aqueous solutions and swells to an equilibrium value, retaining a significant portion of the aqueous solution within its structure. Hydrogels have been shown to be suitable for delivery of a number of compounds, including proteins or peptides (Pitt et al., Int. J. Pharm., 59: 173, 1990.

Additional carriers or excipients for dermal delivery of a compound are described in, for example, Kikwai et al., AAPS Pharm Sci Tech., 6: E565-72, 2005. For example, a suitable carrier is a hydroxypropyl methylcellulose (HPMC) or a hydroxypropyl cellulose (HPC). Such carriers may be formulated as a liquid, a gel or a cream. Optionally, the carrier additionally comprises n-methyl-2-pyrrolidine (NMP) to enhance uptake of a topical composition therein. Methods for producing topical compositions comprising such carriers or excipients will be apparent to the skilled artisan and/or described in, for example, Kikwai et al., supra.

c) Inhalable Formulations

Pharmaceutical formulations adapted for administration by inhalation include fine particle dusts or mists which may be generated by means of various types of metered dose pressurized aerosols, nebulizers or insufflators. Such formulations are suitable for the treatment of for example, infections of the airways, e.g, a P. aeruginosa infection or a Acinetobacter baumannii infection.

Spray compositions may, for example, be formulated as aerosols delivered from pressurized packs, such as a metered dose inhaler, with the use of a suitable liquified propellant.

Capsules and cartridges for use in an inhaler or, insufflator, for example gelatine, may be formulated containing a powder mix for inhalation of a peptide or analog or derivative of the invention and a suitable powder base such as lactose or starch. Each capsule or cartridge may generally contain between about 1 μg and 10 mg of a peptide or analog of the invention.

Aerosol formulations are preferably arranged so that each metered dose or “puff' of aerosol contains about 1 μg to about 2000 μg, such as about 1 μg to about 500 μg of a peptide or analog or derivative of the invention. Administration may be once daily or several times daily, for example 2, 3, 4 or 8 times, giving for example 1, 2 or 3 doses each time. The overall daily dose with an aerosol will generally be within the range 10 μg to about 10 mg, such as 100 μg to about 2000 μg.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close to the nose. Suitable formulations wherein the carrier is a liquid, for administration as a nasal spray or as nasal drops, include aqueous or oil solutions of the active ingredient.

The overall daily dose and the metered dose delivered by capsules and cartridges in an inhaler or insufflator will generally be double those with aerosol formulations.

d) Injectable Formulations

Pharmaceutical formulations adapted for parenteral administration include aqueous and non-aqueous sterile injection solutions which contain a peptide or analog or derivative of the invention and optionally, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets. Such injectable formulations are suitable for the treatment of a variety of disorders, such as those that are treated systemically.

Formulation of a peptide or analog or derivative of the present invention in an intravenous lipid emulsion or a surfactant micelle or polymeric micelle (see., e.g., Jones et al., Eur. J. Pharmaceutics Biopharmaceutics 48, 101-111, 1999; Torchilin J. Clin, release 73, 137-172, 2001; both of which are incorporated herein by reference) is preferred.

Sustained release injectable formulations are produced e.g., by encapsulating the peptide or analog or derivative in porous microparticles which comprise a pharmaceutical agent and a matrix material having a volume average diameter between about 1 μm and 150 μm, e.g., between about 5 μm and 25 μm diameter. In one embodiment, the porous microparticles have an average porosity between about 5% and 90% by volume. In one embodiment, the porous microparticles further comprise one or more surfactants, such as a phospholipid. The microparticles may be dispersed in a pharmaceutically acceptable aqueous or non-aqueous vehicle for injection. Suitable matrix materials for such formulations comprise a biocompatible synthetic polymer, a lipid, a hydrophobic molecule, or a combination thereof. For example, the synthetic polymer can comprise, for example, a polymer selected from the group consisting of poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers, derivatives and blends thereof. In a preferred embodiment, the synthetic polymer comprises a poly(lactic acid), a poly(glycolic acid), a poly(lactic-co-glycolic acid), or a poly(lactide-co-glycolide).

Additional Components in Pharmaceutical Formulations

In another example, an antimicrobial peptide or analog or derivative of the invention is formulated in combination with another antimicrobial agent or antibiotic. Combinations of a peptide or analog or derivative of the invention other agents may be useful to allow antibiotics to be used at lower doses due to toxicity concerns, to enhance the activity of antibiotics whose efficacy has been reduced or to effectuate a synergism between the components such that the combination is more effective than the sum of the efficacy of either component independently. Antibiotics that may be combined with an antimicrobial peptide in combination therapy include but are not limited to penicillin, ampicillin, amoxycillin, vancomycin, cycloserine, bacitracin, cephalolsporin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, and ethambutol.

Other Formulations

In another embodiment, a peptide or analog or derivative of the invention is formulated in a disinfecting or preservative composition, e.g., for cleaning a surface and/or for preserving food or pharmaceuticals. Such a composition comprises a suitable carrier, such as, for example, as described supra. Such a composition also preferably comprises one or more protease inhibitors to reduce or prevent degradation of the antimicrobial peptide of the invention.

In another embodiment, the composition is a phytoprotective composition. Such a composition is, for example, sprayed onto or applied to a plant or soil in which a plant is grown or is to be grown to prevent a microbial infection or to treat a microbial infection.

As will be apparent to the skilled artisan based on the foregoing, a preferred composition is suitable for spray application. For example, the composition is suitable for spraying onto a food product or onto a food preparation surface or onto a plant. Such spray compositions are useful for the treatment of food, e.g., to prevent food spoilage without actually handling the food. The skilled artisan will be aware of suitable components of a composition suitable for spray application. For example the composition comprises an antimicrobial peptide or analog or derivative as described herein according to any embodiment and a suitable carrier, e.g., water or saline. Such a composition may also comprise, for example, a surfactant, e.g., Tween 20, preferably, the surfactant does not inhibit or reduce the antimicrobial activity of said peptide, analog or derivative.

In some embodiments, a peptide or analog or derivative described herein according to any embodiment is applied to a surface of a device to prevent microbial proliferation on that surface of the device. The device is, for example, a medical device, which includes any material or device that is used on, in, or through a patient's body in the course of medical treatment (e.g., for a disease or injury). Medical devices include but are not limited to such items as medical implants, wound care devices, drug delivery devices, catheters and body cavity and personal protection devices. The medical implants include but are not limited to urinary catheters, intravascular catheters, dialysis shunts, wound drain tubes, skin sutures, vascular grafts, implantable meshes, intraocular devices, heart valves, prosthetic devices (e.g., hip prosthetics) and the like. Wound care devices include but are not limited to general wound dressings, biologic graft materials, tape closures and dressings, and surgical incise drapes. Drug delivery devices include but are not limited to needles, drug delivery skin patches, drug delivery mucosal patches and medical sponges.

Reducing or Preventing Microbial Growth

The present inventors have demonstrated that the peptides of the present invention are active against a variety of microorganisms. Accordingly, the peptides of the present invention are useful for, for example, preserving food stuff, e.g., by preventing colonization with a microorganism that causes food-poisoning in a subject or a microorganism that causes food-spoilage. For example, an antimicrobial peptide of the invention is useful for preventing colonization by a bacterium, such as, for example, Staphylococcus aureus, Salmonella, Clostridium perfringens, Campylobacter, Listeria monocytogenes, Vibrio parahaemolyticus, Bacillus cereus, and Entero-pathogenic Escherichia coli or a fungus of the genera Aspergillus, Penicillium or Rhizopus.

The antimicrobial peptides of the invention and/or the analogs or derivatives thereof are useful for the treatment of an infection by a microorganism, such as, for example, a virus, a bacterium or a fungus. Organisms against which a peptide, analog or derivative of the invention are active will be apparent to the skilled artisan and include, for example, a virus from a family selected from the group consisting of Astroviridae, Caliciviridae, Picornaviridae, Togaviridae, Flaviviridae, Caronaviridae, Paramyxviridae, Orthomyxoviridae, Bunyaviridae, Arenaviridae, Rhabdoviridae, Filoviridae, Reoviridae, Bornaviridae, Retroviridae, Poxviridae, Herpesviridae, Adenoviridae, Papovaviridae, Parvoviridae, Hepadnaviridae, (eg., a virus selected from the group consisting of a Coxsackie A-24 virus Adenovirus 11, Adenovirus 21, Coxsackie B virus, Borna Diease Virus, Respiratory syncytial virus, Parainfluenza virus, California encephalitis virus, human papilloma virus, varicella zoster virus, Colorado tick fever virus, Herpes Simplex Virus, vaccinia virus, parainfluenza virus 1, parainfluenza virus 2, parainfluenza virus 3, dengue virus, Ebola virus, Parvovirus B19 Coxsackie A-16 virus, HSV-1, hepatitis A virus, hepatitis B virus, hepatitis C virus, hepatitis D virus, hepatitis E virus, human immunodeficiency virus, Coxsackie B1-B5, Influenza viruses A, B or C, LaCross virus, Lassavirus, rubeola virus Coxsackie A or B virus, Echovirus, lymphocytic choriomeningitis virus, HSV-2, mumps virus, Respiratory Synytial Virus, Epstein-Barr Virus, Poliovirus Enterovirus, rabies virus, rubivirus, variola virus, WEE virus, Yellow fever virus and varicella zoster virus).

Preferably, the peptide is useful for the treatment of an infection by a bacterium, such as for example, a gram-positive bacterium or a gram-negative bacterium. For example, the present invention is useful for treating an infection by a bacterium, such as, for example, S. pyrogenes, S. agalactiae, S. equi, S. canis, S. bovis, S. equinus, S. anginosus, S. sanguis, S. salivarius, S. mitis, S. mutans, S. pyogenes, Enterococcus faecalis, Enterococcus faecium, Staphylococcus, epidermidis, Staphylococcus aureus, Hemophilus influenzae, Pseudomonas aeruginosa, Pseudomonas pseudomallei, Pseudomonas mallei, Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, Listeria monocytogenes, Nocardia asteroides, Bacteroides species, Actinomycetes species, Treponema pallidum, Leptospirosa species, Klebsiella pneumoniae, Escherichia coli, Proteus, Serratia species, Acinetobacter, Yersinia pestis, Francisella tularensis, Enterobacter species, Bacteriodes species or Legionella species

Preferably, the antimicrobial peptide of the present invention is useful for treating an infection caused by a bacterium such as, for example, E. coli, P aeruginosa, Acinetobacter baumannii, or Salmonella typhimurium.

The antimicrobial peptide of the present invention is preferably also useful for treating an infection caused by a fungus, such as, for example, Aspergillus sp., Dermatophytes, Blastomyces, dermatitidis, Candida sp., Malassezia furfur, Exophiala werneckii, Piedraia hortai, Trichosporon beigelii, Pseudallescheria boydii, Madurella grisea, Histoplasma capsulatum, Sporothrix schenckii, Histoplasma capsulatum T. rubrum, T. interdigitale, T. tonsurans, M. audouini, T. violaceum, M. ferrugineum, T. schoenleinii, T. megninii, T. soudanense, T. yaoundei, M. canis , T. equinum, T. erinacei, T. verrucosum, M. nanum (originating from pigs), M. distortum, M. gypseum or M. fulvum

In addition, the invention is useful for controlling protozoan or macroscopic infections by organisms such as Cryptosporidium, Isospora belli, Toxoplasma gondii, Trichomonas vaginalis, Cyclospora species.

Accordingly, an antimicrobial peptide or analog or derivative thereof is useful for treating a condition such as, for example, an infection of the skin and/or an infection of the urogenital tract and/or an infection of the digestive system (e.g., the gut) and/or an infection of the lung, and/or an infection of the sinus. For example, the antimicrobial peptide is useful for the treatment of a condition, such as, for example, rosacea, atopic dermatitis (e.g., eczema), a Candida infection (e.g., vaginal, diaper, intertrigo, balanitis, oral thrush), Tinea versicolor, Dermatophytosis (e.g., Tinea pedis (athlete's foot), Tinea unguium, Onychomycosis (e.g., toe nail fungus), Tinea cruris, Tinea capitus, Tinea corporis, Tinea barbae, seborrheic dermatitis, antibiotic-resistant skin infections, impetigo, ecthyma, erythrasma, burn wounds (e.g., reduction of infections, improved healing), diabetic foot/leg ulcers (e.g., reduction of infections, improved healing), prevention of central catheter-related blood stream infections, oral mucositis, warts (e.g., common, flat, plantar, genital), and molluscum contagiosum. In some embodiments, the condition is acne, often acne vulgaris and sometimes acne conglobate.

The peptides, analogs and/or derivatives of the present invention are also useful for treating a medical condition or a microorganism-causing complication of a medical condition, such as, for example, pneumonia, sepsis or a microbial complication of cystic fibrosis.

Alternatively, or in addition, an antimicrobial peptide of the invention is useful for treating or preventing an infection in a plant, such as, for example, an infection caused by Alternaria spp.; Armillaria mellae; Arthrobotrys oligosporus; Boletus granulatus; Botrytis fabae; Botritis cinerea; Candida albicans; Claviceps purpurea; Cronartium ribicola; Epicoccum purpurescens; Epidermophyton floccosum; Fomes annosus; Fusarium oxysporum; Gaeumannomyces graminis var. tritici; Glomerella cingulata; Gymnosporangium juniperi-virginianae; Microsporum canis; Monilinia fructicola; Physoderma alfalfae; Phytopthera infestans; Pityrosporum orbiculare (Malassezia furfur); Polyporus sulphureus; Puccinia spp.; Saccharomyces cerevisiae; Septoria apiicola; Trichophyton rubrum; T. mentagrophytes; Ustilago spp.; Venturia inaequalis; or Verticillium dahliae.

Methods of Administration or Application

There are numerous application for the present invention, such as treatment of a water sample, a food product or an animal feed. For example, a peptide of the present invention is readily administered to a water supply, a food product, an animal feed or crops, simply by adding the peptide to the water supply, food product, animal feed or crops. As discussed herein, the peptide may be added to a water supply, a food product, animal feed or with a suitable carrier in e.g., a solid, liquid, gel, foam or aerosol form.

In the case of administration to an animal or a human, numerous methods of administering an effective amount of an isolated peptide of the present invention or an analog or derivative thereof are available for use by the skilled artisan. Such isolated peptides may be introduced topically (e.g., in the form of a cream or a spray or a powder), parenterally, transmucosally, e.g., orally, nasally, or rectally, or transdermally, intra-arterially, intramuscularly, intradermally, subcutaneously, intraperitoneally, intraventricularly, and intracranially. Such administration can also occur via bolus administration. Oral solid dosage forms are described generally in Remington's Pharmaceutical Sciences, 18th Ed.1990 (Mack Publishing Co. Easton Pa. 18042) at Chapter 89, and; Marshall, K. In: Modern Pharmaceutics Edited by G. S. Banker and C. T. Rhodes Chapter 10, 1979, both incorporated herein by reference. A method and composition for pulmonary delivery of drugs for systemic effect is described in U.S. Pat. No. 5,451,569, issued Sep. 19, 1995 to Wong et al. (incorporated herein by reference). Systems of aerosol delivery, such as the pressurized metered dose inhaler and the dry powder inhaler are disclosed in Newman, S. P., Aerosols and the Lung, Clarke, S. W. and Davia, D. editors, pp. 197-22 and can be used in connection with the present invention.

In another embodiment, an isolated peptide of the present invention, or variant thereof, can be delivered in a vesicle, in particular a liposome (see Langer, Science 249:1527-1533 (1990); Treat et al., in Liposomes in the Therapy of infectious Disease and Cancer,

In the case of administration to a plant, the peptide of the invention is, for example, sprayed onto a plant or plant part or soil comprising a plant or in which a plant is to be grown. Alternatively, the peptide is a component of a fertilizer to be administered to a plant. Alternatively, the peptide is administered in the form of a powder.

Suitable methods of administration and/or application in other situations will be apparent to the skilled artisan. For example, to apply a peptide, analog or derivative of the invention to a food product or a fluid, the peptide, analog or derivative may be sprayed onto or into said food product or fluid or applied to a container in which the food product or fluid is stored.

The present invention is described further in the following non-limiting examples.

### EXAMPLE 1

### Isolation of Clones Expressing Antimicrobial Peptides

Rationale

This example describes methods developed by the present inventors for isolating clones expressing antimicrobial peptides encoded by genomic DNA fragments of diverse prokaryotes and eukaryotes having compact genomes, wherein the DNA fragments have been size-selected to thereby enhance the likelihood that the encoded peptides form stable secondary structures or assemblies of secondary structures e.g., protein domains or folds, and wherein the encoded peptides do not necessarily exhibit antimicrobial activity in their native contexts.

Methods

As a source of antimicrobial clones, the inventors screened a phage T7 display library produced essentially as described in International Patent Application No. PCT/AU2004/000214 (International Publication No. WO 2004/074479), according to the following protocols.

In general, to isolate phage clones expressing antimicrobial peptides, various modifications of a subtractive phage display process (e.g., Bishop-Hurley et al., Antimicrob. Agents Chemother. 49: 2972-2978, 2005) were developed and employed. Briefly, a T7 phage display library is optionally preadsorbed to one or more of A. lwoffii, Pasteurella pneumotropica and Staphylococcus aureus, and those clones that do not bind are screened in a process comprising three to five rounds of screening by contacting with Acinetobacter spp., e.g., A. baumannii and/or A. lwoffii subject to the proviso that a preadsportion and screening are not conducted using the same bacterium, and the phage clones that bind to Acinetobacter spp., in each round are retained. Optionally, the clones that bind to Acinetobacter spp., in each round are purified further by contacting the clones with one or more of Pasteurella pneumotropica and Staphylococcus aureus, and retaining those clones that bind to one or more of A. baumannii, A. lwoffii and Staphylococcus aureus at a higher affinity than to Pasteurella pneumotropica and/or bovine serum albumin. Optionally, the phage lysate obtained between each round of screening is subjected to precipitation e.g., using polyethylene glycol (PEG).

a) Preadsorption of Library to Non-Target Bacterial Cell Suspensions

For preadsorption, non-target bacterial cells are passed through a 19 G syringe needle three times, collected by centrifugation, washed in Tris-buffered saline (TBS), and resuspended in TBS. Then, approximately 8 ml of T7 phage are combined with 1 ml of the washed bacterial cell suspension containing approximately 1×10¹⁰ c.f.u. of cells (as determined by conventional plate assay) in TBS containing 0.01% (w/v) sterile gelatine, and the suspension is incubated for approximately 1 hour at room temperature with continuous mixing. Cells with phage pre-adsorbed thereto are collected by centrifugation for 10 min at 2,000×g and the pellets discarded. The supernatants are transferred to fresh 15 ml Falcon tubes and stored at 4° C. until required.

b) Biopanning Using Target Bacterial Cell Suspension

Target bacterial cells are passed through a 19 G syringe needle three times, collected by centrifugation, washed in Tris-buffered saline (TBS), and resuspended in TBS. A 1 ml suspension of target bacterial cells (approx. 1×10¹⁰ c.f.u./ml of cells, as determined by conventional plate assay) is then added to approximately 10 ml phage library and incubated at 4° C. overnight with continuous mixing. Cells are collected by centrifugation for 5 min at 2,000×g, and the supernatant discarded. The cell pellet is resuspended gently in 1 ml of TBS by vortexing, and transferred into an Eppendorf tube. The cells are again collected by centrifugation for 5 min at 2000×g, and this washing process repeated twice. The bound phage are eluted from the washed cell pellet, by adding 200 μl of 1% sterile SDS, and shaking the samples at 1400 rpm (Eppendorf mixer) for 10 min. The cells are recovered by centrifugation at maximal speed using an Eppendorf bench-top centrifuge, and discarded. The supernatants are recovered (approximately 200 μl) and used to infect 40 ml of a fresh culture of E. coli BLT5615 cells, and the cells are cultured with continuous shaking at 37° C. until lysis is observed. The lysates are clarified using standard procedures e.g., by centrifugation at 4° C., 6000 rpm for 30 min.

If PEG precipitation is not performed between screening rounds, cleared lysate (approximately 8 ml), is mixed with 1 ml suspension of washed target bacterial cells (approx. 1×10¹⁰ c.f.u./ml of cells) in TBS containing 0.1% (w/v) BSA (or 0.05% (w/v) gelatine) in a final volume of 10 ml, and samples are processed as for the primary round. At the conclusion of the screening, the complete lysate (40 ml) is stored at 4° C. until required.

If PEG precipitation is performed between screening rounds, cleared lysate (approximately 29 ml), is mixed with 2.9 ml of 5M NaCl and 4.8 ml 50% PEG, and the mixtures incubated at 4° C. for approximately 30 min to precipitate the bacteriophage. The phage are recovered by centrifugation at 4° C., 10,000 rpm (Beckman SS34 rotor) for 30 min, and resuspended in 2 ml of 1× TBS. The remaining 11 ml lysate is stored at 4° C. until required. In each subsequent round of screening, 2 ml of PEG-precipitated phage lysate is mixed with 1 ml suspension of washed target bacterial cells (approx. 1×10¹⁰ c.f.u./ml of cells) in TBS containing 0.1% (w/v) BSA (or 0.05% (w/v) gelatine) in a final volume of 10 ml, and samples are processed as for the primary round. At the conclusion of the screening, the complete lysate (40 ml) is stored at 4° C. until required.

A brief summary of various modifications to the generic process tested by the present inventors is provided in Table 1 hereof.

The convention using for naming phage clones isolated using biopanning protocols 1-5 was according to the general form AcXrYcZ, wherein X is the number of the biopanning protocol employed in accordance with Table 1, wherein Y is the number of screening rounds employed, and wherein Z in the clone number. For example, the name Ac2r5c5 refers to clone number 5 isolated following 5 screening rounds using protocol 2 listed in Table 1. The convention using for naming phage clones isolated using biopanning protocol 6 was according to the same general form followed by the suffix “/S” to indicate preadsorption using S. aureus, or alternatively, the general form Ac6X/Y, wherein x is an alphanumeric clone identifier and wherein Y is either L (indicative of preadsorption using A. lwoffii) or S (indicative of preadsorption using S. aureus).

c) Binding Assays

Individual clones isolated by biopanning as described herein were tested for binding to target and non-target bacteria inter alia using a modified ELISA i.e., T7 tailfiber ELISA. Briefly, microtiter dishes were coated overnight or longer with bacterial protein extracts at a concentration of 100 ng-500 ng per well in bicarbonate buffer pH 9.6. The protein was obtained by treatment with formaldehyde, or by sonication. For most purposes, bacterial protein extracts employed were derived from A. baumannii, A. lwoffii, Staphylococcus aureus, Pasteurella pneumotropica, or Salmonella typhimurium. BSA was utilized as a negative control in place of a bacterial protein extract. Following coating, plates were washed three times using PBS buffer containing 0.05% (w/v) Tween-20 and lacking sodium azide (i.e., PBS-T buffer). Wells were then coated for 1 hour at room temperature using PBS-T buffer containing 3% (w/v) BSA, and washed four times using PBS-T buffer. Approximately 50 μl of T7 phage lysate (approx. 1-5×10⁹ p.f.u./well) and 50 μl of PBS-T buffer containing 3% (w/v) BSA was added to each well, and the plates incubated for 2 hours at room temperature with shaking. The wells were then washed five times using PBS-T buffer. To detect bound phage, 100 μl of a 1 mg/ml stock solution of anti-T7 tailfiber mAb (Novagen) diluted 1:2000 (v/v) in PBS-T buffer containing 3% (w/v) BSA was added to wells, and the plates incubated for 1 hour at room temperature. Plates were washed 6 times using PBS-T buffer, and 100 μl of a 1:1500 (v/v) dilution of anti-mouse Ig conjugated to horseradish peroxidase (HRP) in PBS-T buffer containing 1% (w/v) BSA (Amersham) was added. Plates were incubated for a further 1 hour and washed 7 times using PBS-T buffer. Plates were then developed by addition of 100 μl of K-Blue TMB substrate for 20 mins at room temperature. Colour development was stopped by addition of 50 μl of 1M sulfuric acid. Plates were then read in a plate reader at 450 nm.

Results

A brief summary of the binding activities of the phage clones isolated using various modifications to the generic process is provided in Table 1 hereof and explained in the following paragraphs with reference to an example employing A. baumannii or A. lwoffii as the screening target.

Surprisingly, clones isolated using A. baumannii as target in protocol 1 (Table 1) showed equally high or considerably higher binding to A. lwoffii than to A. baumannii irrespective of whether or not the library was preadsorbed to A. lwoffii cells. This suggested shared dominant epitopes between the two bacterial species. The binding to target A. baumannii was not strong for these clones compared to their binding to BSA, and no clones from this procedure were taken forward.

The inventors sought to enhance the selection of phage binding at higher affinity to target A. baumannii cells, by preadsorbing the phage library with a higher concentration of A. lwoffii cells, in addition with P. pneumotropica cells. After five rounds of biopanning according to protocols 2 and 3 (Table 1), approximately 10% of clones that were isolated exhibited medium to strong binding to Acinetobacter spp., relative to their binding to BSA, however all of the isolated positive phage clones bound equally well or even stronger to A. lwoffii than to A. baumannii. This confirmed previous data suggesting shared dominant epitopes between the two bacterial species. Certain clones obtained from protocol 3 were also tested for their binding to S. aureus and P. pneumotropica, and were shown to bind more strongly on average to the gram-positive S. aureus compared to either Acinetobacter spp., albeit more weakly on average to P. pneumotropica.

The inventors also sought to enhance the proportion of clones binding to A. baumannii target or A. lwoffii target, by omitting the prebsorption step in protocols 4 and 5 (Table 1). The effect of omitting preadsorption was significant when A. lwoffii was used as the target, since protocol 4 resulted in a frequency of about 17% of isolated clones having comparable binding activities to A. lwoffii and A. baumannii. The effect of omitting preadsorption was also significant when A. baumannii was used as the target, since protocol 5 resulted in a frequency of about 50% of isolated clones having comparable binding activities to A. lwoffii and A. baumannii. There was no discernible difference between phage clones isolated after round 4 and round 5 in protocol 5, suggesting that the enrichment had occurred in or before the fourth round of biopanning. Certain clones obtained from protocols 4 and 5 without any pre-adsoption or post-adsorption step were also tested for their binding to S. aureus and P. pneumotropica, and were shown to bind more strongly on average to the gram-positive S. aureus compared to either Acinetobacter spp., albeit more weakly on average to P. pneumotropica.

The inventors also tested the effect of adsorption with 5.5×10⁹ c.f.u. S. aureus and 3.5×10¹⁰ c.f.u. P. pneumotropica following the round 5 screening according to protocol 5 i.e., using A. baumannii as target (Table 1). A total of eight bacterial binders were isolated with at least six clones showing a higher specificity for A. baumannii. In general, the clones obtained from protocol 5 following such post-adsorption were also shown to bind more strongly on average to A. baumannii than to the gram-positive S. aureus or P. pneumotropica.

Based in part on the high cross-reactivities of isolated phage clones between A. Baumannii, S. aureus and A. lwoffii, the inventors also tested the effects of pre-adsorption using the non-targets S. aureus and A. lwoffii separately at a higher concentration than previously i.e., 1×10¹⁰ c.f.u./ml, in enhancing recovery of clones binding to an A. baumannii target. Using protocol 6 (Table 1), the inventors isolated 551 phage clones binding to the A. baumannii target, of which 17.1% showed enhanced binding to the target. Preadsorption to S. aureus resulted in the isolation of 22.6% of clones binding to A. baumannii at higher affinity, and preadsorption to A. lwoffii resulted in the isolation of 11.6% of clones binding to A. baumannii at higher affinity. These data suggested that the preadsorption to a relatively high concentration of S. aureus cells is preferred for isolating clones binding to A. baumannii target.

In summary, from six distinct screening protocols employing A. baumannii or A. lwoffii as target, a total of 1442 phage clones were purified and analysed for binding to A. baumannii, of which approximately 140 clones were considered to bind positively i.e., above background. This represents a positive hit rate approximating 10%. The average background signal for binding to BSA was only about 5% of the signal for binding to target.

### EXAMPLE 2

### Sequencing of Positive Clones and Isolation of Antimicrobial Peptides there From

This example demonstrates the identification and isolation of minimal antimicrobial peptides from phage clones obtained as described in Example 1, including multiple peptides having different antimicrobial specificities from the same phage clones.

Methods

a) Sequence Determination

The display peptides of 132 phage clones that were characterized as positive for binding to A. baumannii and/or A. lwoffii as described in Example 1 were sequenced by conventional means and the sequences of the longest open reading frames in the correct orientation determined. The sequences of the encoded peptides were derived by translation in silico of these open reading frames.

b) Design and Analysis of Antimicrobial Peptides

The derived amino acid sequences of the phage clone display peptides were analysed for potential secondary structure (α-helices, β-sheets and random-coil structures), and this information was used to select internal regions of 23 amino acids in length, on average, that were predicted to form a secondary structure. These data were used to design and synthesize “PepSets” of 23-mer peptides for subsequent testing. Where possible, the peptides were designed with an overlap of eight to ten amino acids, with care being taken not to disrupt potential a-helical regions and β-sheets. In cases where multiple peptides were derived from the same clone, one or more consensus sequences were derived corresponding to the region of overlapping sequence, for subsequent correlation to the antimicrobial activities determined for the peptide fragments. Peptides were also analysed using the software of the University of Nebraska for prediction of helix formation, and propensity for interaction with cell membranes.

Peptide analogs consisting of amidated peptides were also produced from the Pepset peptides, wherein the C-terminal residue was α-amidated e.g., to reduce turnover.

Peptide analogs were also produced from the Pepset peptides, wherein multiple cysteine (C) residues in any peptide were substituted for serine (S) residues, to minimize intramolecular disulfide bridge formation.

Peptide analogs consisting of retroinverted forms of certain Pepset peptides were also produced. In general, these were produced for peptides having stronger binding to A. baumannii e.g., as shown in Example 3 below.

Control antimicrobial peptides were also synthesized (Table 2).

Peptides were synthesized to comprise a 5′ biotin-group to allow for efficient detection by Streptavidin or labelled Streptavidin conjugate.

Peptides were dissolved in water or aqueous solution of 10% (v/v) acetonitrile to a final concentration of 1 mM. Reconstitution of the peptides could also be aided by prolonged incubation, addition of acid, DMSO or acetonitrile. Several peptide solutions were treated with ultrasound, incubated at 37° C., and acid was added to reconstitute peptides having high content of basic amino acids.

Results

Sequences of exemplary display peptides from phage clones, Pepset peptides and consensus sequences of overlapping peptides having antimicrobial activity are shown in Table 3 and the accompanying Sequence Listing. Table 3 demonstrates the ontogeny of the Pepset peptides.

Sequences of various analogs of the peptides are presented in Table 4 and the accompanying Sequence Listing.

Predicted secondary structural characteristics for 13 peptides in this study are presented in Table 5. In general, and with the exception of peptide Ac44, the most bioactive peptides comprise about 25-60% hydrophobic amino acid content, and are predicted to form alpha-helices e.g., amphipathic alpha-helices, and to interact with cell membranes. The propensity to form alpha-helices also correlated strongly with predicted antimicrobial activity.

TABLE 1

| Protocol | | |
|---|---|---|
| Number | Brief description | Binding activity of phage clones |
| 1 | With or without preadsorption to | For 20 clones analysed (none taken further): |
| | A. lwoffii followed by three | A. lwoffii ≧ A. baumannii ≧ BSA |
| | rounds of biopanning using | |
| | A. baumannii ATCC 19606 | |
| | with PEG precipitation between | |
| | each biopanning round. | |
| 2 | Peptide library preadsorbed to | For example, clones designated Ac2r5c5, Ac2r5c9, |
| | A. lwoffii and Pasteurella | Ac2r5c34, Ac2r5c38: |
| | pneumotropica, followed by five | A. lwoffii ≧ A. baumannii > BSA |
| | rounds of biopanning using | |
| | A. baumannii ATCC 19606 | |
| | without PEG precipitation | |
| | between biopanning rounds. | |
| 3 | Peptide library preadsorbed to | For example, clones designated Ac3r5c8, Ac3r5c10: |
| | A. lwoffii and Pasteurella | S. aureus ≧ A. lwoffii > A. baumannii ≧ |
| | pneumotropica, followed by five | P. pneumotropica > BSA |
| | rounds of biopanning using | |
| | A. baumannii ATCC 19606 or | |
| | Acinetobacter sp. ATCC 33305 | |
| | with PEG precipitation between | |
| | each biopanning round. | |
| 4 | No preadsorption, followed by | For example, clones designated Ac4r5c1, Ac4r5c10, |
| | five rounds of biopanning using | Ac4r5c59, Ac4r5c68, Ac4r5c71: |
| | A. lwoffii and PEG precipitation | S. aureus ≧ A. lwoffii ≧ A. baumannii ≧ |
| | between each biopanning round. | P. pneumotropica > BSA |
| 5 | No preadsorption, followed by | No post-adsorption, for example, clones designated |
| | four or five rounds of | Ac5r4c2, Ac5r4c23, Ac5r4c39, Ac5r4c54, Ac5r4c57, |
| | biopanning using A. baumannii | Ac5r4c60, Ac5r4c71, Ac5r5c39, Ac5r5c79: |
| | ATCC 19606 with PEG | S. aureus ≧ A. lwoffii ≧ A. baumannii ≧ |
| | precipitation between each | P. pneumotropica > BSA |
| | biopanning round, and optional | With post-adsorption, for example clones designated |
| | post-adsorption to | Ac5r5c50, Ac5r5c56, Ac5r5c69, Ac5r5c80, Ac5r5c111 |
| | Staphyococcus aureus and | and Ac5r5c50c117: |
| | Pasteurella pneumotropica. | A. baumannii ≧ P. pneumotropica ≧ S. aureus > BSA |
| 6 | Preadsorption to S. aureus or | Relative binding to different microorganisms not |
| | A. lwoffii followed by five rounds | compared for phage clones, however 17.1% of clones |
| | of biopanning using | overall showed increased binding to A. baumannii; and |
| | A. baumannii and PEG | 22.6% of phage clones preadsoprbed to S. aureus showed |
| | precipitation between each | increased binding activity to A. baumannii; and 11.6% of |
| | biopanning round. | clones preadsorbed with A. lwoffii showed increased |
| | | binding to A. baumannii. |

TABLE 2
Control antimicrobial peptides

| | Length | |
|---|---|---|
| Name | [aa] | Description |
| SRCRP2 | 16 | Scavenger receptor cysteine-rich peptide fragment from salivary |
| (SEQ ID NO: 172) | | agglutinin. Binds to a variety of Gram+ and Gram− bacteria incl. |
| | | S. aureus Bikker et al., J. Biol. Chem, 277: 32109-32115, 2002) |
| hi3/17 | 15 | Display peptide of M13 phage binding to non-typeable H. influenzae |
| (SEQ ID NO: 173) | | (Bishop-Hurley et al., supra) |
| Tachyplesin-1 | 17 | Highly basic antimicrobial peptide from Japanese horseshoe crab. |
| (SEQ ID NO: 174) | | Contains two disulfide bridges. Active against Gram+ and Gram− |
| | | bacteria (Nakamura et al., J. Biol. Chem., 263: 16709-16713, 1988) |
| Magainin-2 | 23 | Identified from African clawed frog; active against Gram+ and Gram− |
| (SEQ ID NO: 175) | | bacteria and fungi (Gesell et al., J. Biomol. NMR, 9: 127-135, 1997) |
| Brevinin-1EB | 23 | Antimicrobial peptide from Rana esculenta. Active against Gram+ and |
| (SEQ ID NO: 176) | | Gram− bacteria and mammalian cells (Simmaco et al., J. Biol. Chem, |
| | | 269: 11956-11961, 1994) |
| Aurein 1.1 | 13 | Identified from southern bell frog; active against Gram+ and Gram− |
| (SEQ ID NO: 177) | | bacteria and fungi (Rozek et al., Eur. J. Biochem., 267: 5330-5341, 2000) |

TABLE 3

| Phage | Display peptide | Pepset peptide(s) | Consensus sequences of peptides |
|---|---|---|---|
| Clone | SEQ ID NO: | (SEQ ID NO.) | (SEQ ID NO.) |
| Ac2r5c5 | SEQ ID NO: 1 | Ac3 (SEQ ID NO: 2) | None |
| | | Ac5 (SEQ ID NO: 3) | |
| Ac2r5c9 | SEQ ID NO: 4 | Ac8 (SEQ ID NO: 6) | SEQ ID NO: 5 |
| | | Ac9 (SEQ ID NO: 7) | |
| Ac2r5c34 | SEQ ID NO: 8 | Ac13 (SEQ ID NO: 10) | SEQ ID NO: 9 |
| | | Ac15 (SEQ ID NO: 12) | |
| Ac2r5c38 | SEQ ID NO: 14 | Ac17 (SEQ ID NO: 16) | SEQ ID NO: 15 |
| | | Ac18 (SEQ ID NO: 17) | |
| Ac3r5c8 | SEQ ID NO: 19 | Ac35 (SEQ ID NO: 20) | |
| Ac3r5c10 | SEQ ID NO: 21 | Ac38 (SEQ ID NO: 23) | SEQ ID NO: 22 |
| | | Ac40 (SEQ ID NO: 25) | |
| Ac4r5c1 | SEQ ID NO: 27 | Ac44 (SEQ ID NO: 28) | |
| Ac4r5c10 | SEQ ID NO: 29 | Ac46 (SEQ ID NO: 31) | SEQ ID NO: 30 |
| | | Ac47 (SEQ ID NO: 32) | |
| Ac4r5c59 | SEQ ID NO: 33 | Ac53 (SEQ ID NO: 34) | |
| Ac4r5c68 | SEQ ID NO: 35 | Ac56 (SEQ ID NO: 36) | |
| Ac4r5c71 | SEQ ID NO: 37 | Ac59 (SEQ ID NO: 38) | |
| Ac5r4c2 | SEQ ID NO: 39 | Ac66 (SEQ ID NO: 43) | SEQ ID NO: 40 (Ac66/Ac67/Ac68) |
| | | Ac67 (SEQ ID NO: 44) | SEQ ID NO: 41 (Ac66/Ac67) |
| | | Ac68 (SEQ ID NO: 45) | SEQ ID NO: 42 (Ac67/Ac68) |
| Ac5r4c23 | SEQ ID NO: 46 | Ac88 (SEQ ID NO: 47) | |
| Ac5r4c39 | SEQ ID NO: 48 | Ac96 (SEQ ID NO: 49) | |
| Ac5r4c54 | SEQ ID NO: 51 | Ac97 (SEQ ID NO: 55) | SEQ ID NO: 52 (Ac97/Ac98) |
| | | Ac98 (SEQ ID NO: 56) | SEQ ID NO: 53 (Ac97/Ac99) |
| | | Ac99 (SEQ ID NO: 57) | SEQ ID NO: 54 (Ac99/Ac100) |
| | | Ac100 (SEQ ID NO: 58) | |
| Ac5r4c57 | SEQ ID NO: 59 | Ac101 (SEQ ID NO: 60) | |
| Ac5r4c60 | SEQ ID NO: 61 | Ac102 (SEQ ID NO: 63) | SEQ ID NO: 102 |
| | | Ac103 (SEQ ID NO: 64) | |
| Ac5r4c71 | SEQ ID NO: 65 | Ac116 SEQ ID NO: 66) | |
| Ac5r5c39 | SEQ ID NO: 67 | Ac138 (SEQ ID NO: 68) | |
| Ac5r5c79 | SEQ ID NO: 69 | Ac173 (SEQ ID NO: 70) | |
| Unknown | Not shown | Ac193 (SEQ ID NO: 71) | |
| Unknown | Not shown | Ac195 (SEQ ID NO: 72) | |
| Ac6r3c13/S | Not shown | Ac228 (SEQ ID NO: 73) | |
| Ac6r3c30/S | Not shown | Ac259 (SEQ ID No; 75) | |
| Ac6b5/S | SEQ ID NO: 77 | Ac319 (SEQ ID NO: 76) | SEQ ID NO: 78 (Ac319/Ac322) |
| | | Ac322 (SEQ ID NO: 80) | SEQ ID NO: 79 (Ac322/Ac323) |
| | | Ac323 (SEQ ID NO: 82) | |
| Ac6b10/S | SEQ ID NO: 84 | Ac327 (SEQ ID NO: 88) | SEQ ID NO: 85 (Ac327/Ac328/Ac329) |
| | | Ac328 (SEQ ID NO: 89) | SEQ ID NO: 86 (Ac327/Ac328) |
| | | Ac329 (SEQ ID NO: 91) | SEQ ID NO: 87 (Ac328/Ac329) |
| Ac6b13/S | Not shown | Ac330 (SEQ ID NO: 93) | |
| Ac6b14/S | SEQ ID NO: 94) | Ac338 (SEQ ID NO: 98) | SEQ ID NO: 95 (Ac338. Ac339/Ac340) |
| | | Ac339 (SEQ ID NO: 99) | SEQ ID NO: 96 (Ac338/Ac339) |
| | | Ac340 (SEQ ID NO: 100) | SEQ ID NO: 97 (Ac339/Ac340) |
| Ac6b33/S | SEQ ID NO: 101 | Ac364 (SEQ ID NO: 103) | None |
| | SEQ ID NO: 102 | Ac370 (SEQ ID NO: 105) | |
| Ac6b11/S | SEQ ID NO: 107 | Ac378 (SEQ ID NO: 111) | SEQ ID NO: 108 (Ac378/AC379/Ac380) |
| | | Ac379 (SEQ ID NO: 113) | SEQ ID NO: 109 (Ac378/Ac379) |
| | | Ac380 (SEQ ID NO: 115) | SEQ ID NO: 110 (Ac379/Ac380) |
| Ac6b29/L | SEQ ID NO: 117 | Ac389 (SEQ ID NO: 119) | SEQ ID NO: 118 |
| | | Ac390 (SEQ ID NO: 120) | |
| Ac6c19/L | Not shown | Ac431 (SEQ ID NO: 121) | |
| Ac6c28/L | Not shown | Ac434 (SEQ ID NO: 123) | |
| Ac6c28/L | SEQ ID NO: 124 | Ac436 (SEQ ID NO: 126) | SEQ ID NO: 125 |
| | | Ac437 (SEQ ID NO: 127) | |
| Ac6c31/L | Not shown | Ac451 (SEQ ID NO: 128) | |
| Ac6d13/S | Not shown | Ac469 (SEQ ID NO: 130) | |
| Ac6d18/S | Not shown | Ac472 (SEQ ID NO: 131) | |
| Ac6d31/S | SEQ ID NO: 133 | Ac474 (SEQ ID NO: 137) | SEQ ID NO: 134 (Ac474/Ac475/Ac476) |
| | | Ac475 (SEQ ID NO: 138) | SEQ ID NO: 135 (Ac474/Ac475) |
| | | Ac476 (SEQ ID NO: 139) | SEQ ID NO: 136 (Ac475/Ac476) |
| Ac6e32/L | Not shown | Ac496 (SEQ ID NO: 140) | |

TABLE 4

| Pepset peptide(s) | Serine analogs | Retroinverso peptide analog |
|---|---|---|
| (SEQ ID NO.) | (SEQ ID NO.) | (SEQ ID NO.) |
| Ac3 (SEQ ID NO: 2) | None | None |
| Ac5 (SEQ ID NO: 3) | None | Ac315 (SEQ ID NO: 141) |
| Ac8 (SEQ ID NO: 6) | None | SEQ ID NO: 142 |
| Ac9 (SEQ ID NO: 7) | None | None |
| Ac13 (SEQ ID NO: 10) | Ac14 = Ac13-C11S (SEQ ID NO: 11) | Ac205 (SEQ ID NO: 143-144) |
| Ac15 (SEQ ID NO: 12) | Ac16 = Ac15-C16S (SEQ ID NO: 13) | None |
| Ac17 (SEQ ID NO: 16) | None | SEQ ID NO: 145 |
| Ac18 (SEQ ID NO: 17) | Ac19 = Ac18-C14S/C16S/C19S(SEQ ID NO: 18) | None |
| Ac35 (SEQ ID NO: 20) | None | SEQ ID NO: 146 |
| Ac38 (SEQ ID NO: 23) | Ac39 = Ac38-C8S/C12S/C19S (SEQ ID NO: 24) | None |
| Ac40 (SEQ ID NO: 25) | Ac41 (SEQ ID NO: 26) | None |
| Ac44 (SEQ ID NO: 28) | None | SEQ ID NO: 147 |
| Ac46 (SEQ ID NO: 31) | None | None |
| Ac47 (SEQ ID NO: 32) | None | None |
| Ac53 (SEQ ID NO: 34) | None | SEQ ID NO: 148 |
| Ac56 (SEQ ID NO: 36) | None | None |
| Ac59 (SEQ ID NO: 38) | None | SEQ ID NO: 149 |
| Ac66 (SEQ ID NO: 43) | None | None |
| Ac67 (SEQ ID NO: 44) | None | SEQ ID NO: 150 |
| Ac68 (SEQ ID NO: 45) | None | None |
| Ac88 (SEQ ID NO: 47) | None | None |
| Ac96 (SEQ ID NO: 49) | Ac96-C5S/C14S (SEQ ID NO: 50) | None |
| Ac97 (SEQ ID NO: 55) | None | None |
| Ac98 (SEQ ID NO: 56) | None | None |
| Ac99 (SEQ ID NO: 57) | None | None |
| Ac100 (SEQ ID NO: 58) | None | None |
| Ac101 (SEQ ID NO: 60) | None | None |
| Ac102 (SEQ ID NO: 63) | None | None |
| Ac103 (SEQ ID NO: 64) | None | None |
| Ac116 SEQ ID NO: 66) | None | None |
| Ac138 (SEQ ID NO: 68) | None | None |
| Ac173 (SEQ ID NO: 70) | None | None |
| Ac193 (SEQ ID NO: 71) | None | SEQ ID NO: 151 |
| Ac195 (SEQ ID NO: 72) | None | SEQ ID NO: 152 |
| Ac228 (SEQ ID NO: 73) | Ac228-C1S/C2S/C22S (SEQ ID NO: 74) | None |
| Ac259 (SEQ ID No; 75) | None | None |
| Ac319 (SEQ ID NO: 76) | None | None |
| Ac322 (SEQ ID NO: 80) | Ac322-C12S/C23S (SEQ ID NO: 81) | SEQ ID NO: 153-154 |
| Ac323 (SEQ ID NO: 82) | Ac323-C6S/C17S(SEQ ID NO: 83) | None |
| Ac327 (SEQ ID NO: 88) | None | None |
| Ac328 (SEQ ID NO: 89) | Ac328-C4S/C22S/C23S (SEQ ID NO: 90) | SEQ ID NO: 155-156 |
| Ac329 (SEQ ID NO: 91) | Ac329-C15S/C16S (SEQ ID NO: 92) | None |
| Ac330 (SEQ ID NO: 93) | None | SEQ ID NO: 157 |
| Ac338 (SEQ ID NO: 98) | None | None |
| Ac339 (SEQ ID NO: 99) | None | SEQ ID NO: 158 |
| Ac340 (SEQ ID NO: 100) | None | None |
| Ac364 (SEQ ID NO: 103) | Ac364-C5S/C12S/C14S (SEQ ID NO: 104) | None |
| Ac370 (SEQ ID NO: 105) | Ac370-C17S/C19S (SEQ ID NO: 106) | SEQ ID NO: 159-160 |
| Ac378 (SEQ ID NO: 111) | Ac378-C15S/C21S (SEQ ID NO: 112) | SEQ ID NO: 161-162 |
| Ac379 (SEQ ID NO: 113) | Ac379-C9S/C15S/C19S (SEQ ID NO: 114) | None |
| Ac380 (SEQ ID NO: 115) | Ac380-C4S/C10S/C14S (SEQ ID NO: 116) | None |
| Ac389 (SEQ ID NO: 119) | None | SEQ ID NO: 163 |
| Ac390 (SEQ ID NO: 120) | None | None |
| Ac431 (SEQ ID NO: 121) | Ac431-C1S/C8S/C9S (SEQ ID NO: 122) | SEQ ID NO: 164-165 |
| Ac434 (SEQ ID NO: 123) | None | None |
| Ac436 (SEQ ID NO: 126) | None | SEQ ID NO: 166 |
| Ac437 (SEQ ID NO: 127) | None | None |
| Ac451 (SEQ ID NO: 128) | Ac451-C8S/C12S/C13S (SEQ ID NO: 129) | None |
| Ac469 (SEQ ID NO: 130) | None | None |
| Ac472 (SEQ ID NO: 131) | Ac472-C4S/C12S (SEQID NO: 132) | None |
| Ac474 (SEQ ID NO: 137) | None | None |
| Ac475 (SEQ ID NO: 138) | None | None |
| Ac476 (SEQ ID NO: 139) | None | SEQ ID NO: 167 |
| Ac496 (SEQ ID NO: 140) | None | None |

TABLE 5

| | | | Structure/Function predictions | | | |
|---|---|---|---|---|---|---|
| | Percent | Positive | Alpha- | H. Res. | Boman | Anti- |
| Peptide | hydrophobic | residues | helix | Surface | Index | microbial |
| Ac5 | 34% | 6 | — | — | 1.5 | — |
| Ac6 | 36% | 5 | Yes | 4 | 3.3 | Yes |
| Ac13 | 47% | 7 | Yes | 6 | 2.8 | Yes |
| Ac17 | 34% | 4 | Yes | 5 | 2.4 | Yes |
| Ac35 | 30% | 7 | Yes | 3 | 2.1 | Yes |
| Ac38 | 60% | 3 | Yes | 7 | 1.1 | Yes |
| Ac44 | 8% | 1 | — | — | 1.3 | — |
| Ac59 | 47% | 8 | — | — | 2.2 | — |
| Ac67 | 26% | 5 | Yes | 5 | 3.2 | Yes |
| Ac195 | 34% | 4 | Yes | 4 | 2.5 | Yes |
| Ac259 | 34% | 7 | Yes | 6 | 2.2 | Yes |
| Ac322 | 43% | 5 | — | — | 1.4 | Yes |
| Ac476 | 34% | 4 | Yes | 5 | 2.3 | Yes |

### EXAMPLE 3

### Determination of Antimicrobial Spectra and Efficacy of Antimicrobial Peptides

This example demonstrates the antibacterial spectra and efficacies of certain antimicrobial peptides identified by the inventors, as determined by inhibition of bacterial growth for Acinetobacter spp., including A. baumannii ATCC Accession No. 19606), Acinetobacter spp. ATCC 17903, and Acinetobacter spp. ATCC 19004, and for Escherichia coli strain BL21, S. aureus, S. typhimurium strain AroA, P. aeruginosa and P. pneumotropica. Minimum inhibitory concentrations of peptides against A. baumannii and S. aureus were also determined for certain peptides.

Methods

a) Serial Dilution

Synthetic peptides were tested for their ability to inhibit bacterial growth in liquid culture. The peptides were added at a concentration of between about 5 μM and about 10 μM to individual wells of 96-deep-well dishes that contained liquid growth media freshly inoculated with one of the following bacteria: A. baumannii ATCC Accession No. 19606, Acinetobacter spp. ATCC 17903, Acinetobacter spp. ATCC 19004, Escherichia coli strain BL21, S. aureus, S. typhimurium strain AroA, P. aeruginosa or P. pneumotropica. After 5-6 hours of shaking at 37° C., growth was measured as absorbance at 595 nm. As a control, cultures were grown in the presence of 0.1% acetonitrile (i.e., the concentration present in 10 μM peptide).

b) Broth Microdilution Against A. baumannii Complex

Broth dilution methods are e.g., described by Lorian (2007) In: Antibiotics in Laboratory Medicine, Fifth Edition; and In: Clinical and Laboratory Standards Institute, CLSI (formerly NCCLS), In: Methods for Dilution Antimicrobial Susceptibility Tests for Bacteria that Grow Aerobically—Approved Standard—Seventh Edition, CLSI document M7-A7, Villanova, Pa. (NCCLS; January 2006; Vol. 26 No. 2); and In: Clinical and Laboratory Standards Institute, CLSI (formerly NCCLS), Performance Standards for Antimicrobial Susceptibility Testing, Seventeenth Informational Supplement, M100-S17, Vol 27 No. 1 January 2007, all of which are incorporated herein by reference.

The present inventors used such methods to quantitate activity in vitro of antimicrobial peptides of the invention against bacterial isolates, including laboratory strains and 50 clinical isolates of A. baumannii, and S. aureus ATCC Accession No. 2992. Briefly, small volumes of broth are dispensed in sterile, plastic microtiter trays, and 100 μl of serially-diluted antimicrobial peptide (0.1-64 μg) and 100 μl test organism (approximately 1×10⁵ C.F.U.), are added. Following aerobic incubation overnight at 35±2° C. Control samples lacked peptide (Growth control), or lacked peptide and bacteria (Sterility control). The trays were examined visually and the minimal inhibitory concentration (MIC) of peptide required to inhibit bacterial growth were determined. The minimum bactericidal concentration (MBC) was estimated by sub-culturing the wells for which no bacterial growth was visible to the naked eye, to thereby determine a concentration of antimicrobial that reduces the original inoculum by at least about 99.9%.

c) Time Taken to Exert Cytotoxic Effect

To determine the time killing times for bactericidal peptides, A. baumannii cultures were exposed to different concentrations of peptides for predetermined time periods, and the numbers of bacteria surviving at the end of each time period were determined. Peptides were classified as “Fast”, “Intermediate” or “Slow” according to whether they exerted their cytotoxic effect in less than 30 mins, or between 30-60 mins, or between 120 mins and 240 mins.

Results

Data for serial dilutions are shown in Table 6, wherein data indicating greater than 90% inhibition of bacterial growth for any bacterium tested at 20 μM peptide concentration of less are shown in bold font. Data indicate only weak inhibitory activity of any peptides against Escherichia coli strain BL21, S. aureus, S. typhimurium strain AroA, P. aeruginosa and P. pneumotropica, however the positive control peptide tachyplesin-1 inhibited growth of E. coli significantly.

Data presented in Table 6 also indicate that the following peptides selectively inhibited growth of one or more Acenitobacer sp. Selected from A. baumannii ATCC Accession No. 19606, Acinetobacter spp. ATCC 17903 and Acinetobacter spp. ATCC 19004: Ac14, Ac15, Ac16, Ac17, Ac35, Ac59, Ac68, Ac116 and Ac173. Of these, the following peptides selectively inhibited growth of A. baumannii ATCC Accession No. 19606, Acinetobacter spp. ATCC 17903 and Acinetobacter spp. ATCC 19004: Ac14, Ac15, Ac16, Ac17, Ac35; the following peptides selectively inhibited growth of A. baumannii ATCC Accession No. 19606 and Acinetobacter spp. ATCC 17903: Ac59; and the following peptides selectively inhibited growth of Acinetobacter spp. ATCC 19004: Ac68, Ac116 and Ac173. None of the control peptides tested possessed these spectra of antibacterial activities.

Data presented in Table 6 also indicate that the following peptides selectively inhibited growth of A. baumannii ATCC Accession No. 19606, Acinetobacter spp. ATCC 17903, Acinetobacter spp. ATCC 19004, and S. aureus: Ac3, Ac5, Ac8, Ac19, Ac38, Ac39, Ac40. None of the control peptides tested possessed this spectrum of antibacterial activity.

Data presented in Table 6 also indicate that the following peptides selectively inhibited growth of Acinetobacter spp. ATCC 19004, and S. aureus: Ac41, Ac44, Ac46, Ac47, Ac53, Ac66, Ac67, Ac96, Ac99, Ac101 and Ac102. None of the control peptides tested possessed this spectrum of antibacterial activity.

Data presented in Table 6 also indicate that the following peptides selectively inhibited growth of S. aureus: Ac18, Ac56, Ac88, Ac97, Ac98, Ac100, Ac103, and the control peptides Ac175 (SRCRP2) and Ac178 (Magainin-2).

In summary, the data presented in Table 6 indicate that peptides of the present invention that at least inhibit the growth of one or more bacteria of the A. baumannii complex e.g., A. baumannii ATCC Accession No. 19606 and/or Acinetobacter spp. ATCC 17903 and/or Acinetobacter spp. ATCC 19004, possess unique spectra of antimicrobial activities compared to Ac175 (SRCRP2) and/or Ac177 (tachyplesin-1) and/or Ac178 (Magainin-2).

The MIC values of alpha-amidated and non-amidated peptides against A. baumannii are presented in Table 7. An MIC value of 25 μM or less was considered to be indicative of strong antibacterial activity. In general, amidation of peptides enhanced their antibacterial activity as determined by MIC. Retroinverso analogs of peptides also exhibited greater antibacterial activity than their respective parent peptides.

Data showing minimum bactericidal concentration (MBC) relative to MIC for certain peptides having bactericidal activity against A. baumannii are presented in Table 8. Other peptides not listed in Table 8 were also bactericidal e.g., Ac5, Ac13, Ac17, Ac35, Ac38, Ac44, Ac53, Ac59, Ac193 and Ac195. Of the peptides presented in Table 8, peptides designated Ac228, Ac472, Ac319 and Ac469 were shown to be bacteriostatic against A. baumannii, and the remaining peptides tested were cytotoxic. Additionally, peptides designated Ac259, Ac322, Ac323, Ac327, Ac328, Ac378, Ac389, Ac431, Ac436, Ac476, Ac474 and Ac475 had MIC and MBC values of 25 μM or less, indicating that those peptides are stronger cytotoxins against A. baumannii.

Determination of killing times resulted in the classifications of 11 cytotoxic peptides into three groups as follows:

SLOW: Peptides designated Ac44, Ac53 and Ac193 required between about 120 mins and about 240 mins to kill bacteria completely, suggesting a mechanism of action requiring their translocation to within the cell or alternatively, an effect involving inhibition or activation of an intracellular pathway.

INTERMEDIATE: Peptides designated Ac17, Ac35, Ac59, and Ac195 killed cells completely within 30-60 mins.

RAPID: Peptides Ac5, Ac13, Ac38 and Ac259 required less than 10 mins to kill cells completely, and peptides Ac5, Ac13 and Ac259 required less than 5 mins to kill cells completely, comparable with the killing time of the positive control peptide tachyplesin-1, suggesting a mechanism of action involving surface-localized effect(s).

TABLE 6

| | | % inhibition of bacterial growth by peptide | | | | | | | |
|---|---|---|---|---|---|---|---|---|---|
| | Peptide | A. baumannii | Acinetobacter spp. | | | | S. | | |
| | Concentration | ATCC | ATCC | ATCC | S. | E. coli | typhimurium | P. | P. |
| Peptide | (μM) | 19606 | 17903 | 19004 | aureus | BL21 | AroA | aeruginosa | pneumotropica |
| Ac3 | 20 | 93 | 93 | 94 | 95 | 38 | 18 | 5 | 16 |
| | 15 | 34 | 34 | 81 | 98 | 33 | 6 | 0 | 51 |
| | 10 | 0 | 0 | 32 | 79 | 31 | 2 | 0 | 50 |
| | 5 | 0 | 0 | 0 | 30 | 18 | 2 | 1 | 48 |
| Ac5 | 20 | 94 | 94 | 87 | 98 | 36 | 7 | 0 | 0 |
| | 15 | 95 | 95 | 88 | 100 | 20 | 0 | 0 | 40 |
| | 10 | 92 | 92 | 89 | 60 | 26 | 0 | 0 | 53 |
| | 5 | 4 | 4 | 98 | 29 | 7 | 0 | 10 | 17 |
| Ac8 | 20 | 98 | 98 | 99 | 99 | 37 | 32 | 0 | 75 |
| | 15 | 34 | 34 | 98 | 99 | 24 | 19 | 0 | 81 |
| | 10 | 4 | 4 | 32 | 70 | 24 | 4 | 0 | 55 |
| | 5 | 0 | 0 | 22 | 31 | 18 | 3 | 12 | 43 |
| Ac9 | 20 | 7 | 7 | 50 | 3 | 18 | 20 | 0 | 22 |
| | 15 | 8 | 8 | 54 | 2 | 2 | 5 | 0 | 9 |
| | 10 | 8 | 8 | 46 | 10 | 18 | 0 | 0 | 0 |
| | 5 | 0 | 0 | 23 | 23 | 3 | 0 | 0 | 0 |
| Ac13 | 20 | 83 | 83 | 63 | 71 | 23 | 17 | 0 | 0 |
| | 15 | 88 | 88 | 99 | 55 | 23 | 1 | 0 | 0 |
| | 10 | 90 | 90 | 89 | 44 | 22 | 0 | 0 | 3 |
| | 5 | 96 | 96 | 92 | 47 | 21 | 0 | 13 | 29 |
| Ac14 | 20 | 95 | 95 | 90 | 85 | 32 | 10 | 0 | 28 |
| | 15 | 96 | 96 | 91 | 62 | 24 | 1 | 4 | 63 |
| | 10 | 97 | 97 | 92 | 37 | 29 | 0 | 0 | 53 |
| | 5 | 99 | 99 | 98 | 45 | 14 | 0 | 12 | 29 |
| Ac15 | 20 | 80 | 80 | 66 | 19 | 28 | 13 | 0 | 0 |
| | 15 | 85 | 85 | 74 | 20 | 14 | 4 | 5 | 0 |
| | 10 | 90 | 90 | 84 | 27 | 26 | 1 | 10 | 8 |
| | 5 | 95 | 95 | 91 | 29 | 5 | 0 | 9 | 19 |
| Ac16 | 20 | 97 | 97 | 95 | 19 | 17 | 19 | 0 | 53 |
| | 15 | 97 | 97 | 96 | 19 | 1 | 12 | 0 | 44 |
| | 10 | 97 | 97 | 98 | 13 | 16 | 0 | 0 | 13 |
| | 5 | 59 | 59 | 99 | 22 | 3 | 0 | 12 | 0 |
| Ac17 | 20 | 94 | 94 | 86 | 38 | 0 | 15 | 0 | 19 |
| | 15 | 96 | 96 | 97 | 41 | 1 | 4 | 0 | 35 |
| | 10 | 33 | 33 | 80 | 49 | 19 | 0 | 7 | 0 |
| | 5 | 13 | 13 | 97 | 47 | 1 | 0 | 9 | 0 |
| Ac18 | 20 | 59 | 59 | 71 | 97 | 19 | 15 | 5 | 25 |
| | 15 | 23 | 23 | 31 | 94 | 8 | 5 | 5 | 63 |
| | 10 | 0 | 0 | 0 | 50 | 15 | 0 | 0 | 53 |
| | 5 | 0 | 0 | 0 | 32 | 0 | 0 | 19 | 14 |
| Ac19 | 20 | 90 | 90 | 88 | 99 | 18 | 23 | 0 | 6 |
| | 15 | 18 | 18 | 90 | 92 | 11 | 15 | 2 | 70 |
| | 10 | 1 | 1 | 49 | 42 | 12 | 0 | 0 | 55 |
| | 5 | 0 | 0 | 35 | 23 | 7 | 0 | 2 | 26 |
| Ac35 | 20 | 95 | 95 | 92 | 38 | 39 | 7 | 17 | 0 |
| | 15 | 94 | 94 | 87 | 35 | 2 | 1 | 2 | 0 |
| | 10 | 96 | 96 | 92 | 19 | 13 | 0 | 13 | 8 |
| | 5 | 92 | 92 | 97 | 34 | 0 | 0 | 25 | 21 |
| Ac38 | 20 | 94 | 94 | 89 | 94 | 82 | 53 | 96 | 50 |
| | 15 | 96 | 96 | 92 | 94 | 50 | 21 | 41 | 67 |
| | 10 | 97 | 97 | 95 | 97 | 6 | 14 | 0 | 66 |
| | 5 | 3 | 3 | 0 | 98 | 9 | 0 | 5 | 14 |
| Ac39 | 20 | 100 | 100 | 97 | 97 | 74 | 48 | 78 | 78 |
| | 15 | 100 | 100 | 99 | 98 | 49 | 11 | 21 | 86 |
| | 10 | 99 | 99 | 94 | 98 | 30 | 35 | 0 | 79 |
| | 5 | 6 | 6 | 20 | 86 | 5 | 2 | 5 | 64 |
| Ac40 | 20 | 97 | 97 | 94 | 94 | 75 | 51 | 88 | 75 |
| | 15 | 98 | 98 | 96 | 96 | 50 | 30 | 37 | 84 |
| | 10 | 99 | 99 | 96 | 98 | 30 | 45 | 0 | 76 |
| | 5 | 11 | 11 | 34 | 93 | 17 | 0 | 4 | 55 |
| Ac41 | 20 | 73 | 73 | 94 | 96 | 30 | 12 | 0 | 66 |
| | 15 | 23 | 23 | 50 | 98 | 12 | 5 | 0 | 63 |
| | 10 | 2 | 2 | 34 | 76 | 3 | 8 | 0 | 47 |
| | 5 | 1 | 1 | 3 | 21 | 6 | 3 | 0 | 17 |
| Ac44 | 20 | 58 | 58 | 100 | 96 | 28 | 14 | 0 | 88 |
| | 15 | 30 | 30 | 50 | 98 | 10 | 3 | 0 | 84 |
| | 10 | 3 | 3 | 32 | 73 | 12 | 0 | 0 | 68 |
| | 5 | 0 | 0 | 6 | 20 | 8 | 0 | 0 | 45 |
| Ac46 | 20 | 58 | 58 | 96 | 98 | 28 | 36 | 12 | 88 |
| | 15 | 26 | 26 | 42 | 99 | 6 | 18 | 0 | 77 |
| | 10 | 1 | 1 | 20 | 72 | 0 | 16 | 0 | 61 |
| | 5 | 3 | 3 | 9 | 16 | 0 | 3 | 0 | 43 |
| Ac47 | 20 | 47 | 47 | 97 | 97 | 25 | 50 | 0 | 88 |
| | 15 | 24 | 24 | 37 | 96 | 6 | 29 | 0 | 81 |
| | 10 | 0 | 0 | 25 | 65 | 6 | 25 | 0 | 63 |
| | 5 | 3 | 3 | 5 | 22 | 0 | 13 | 0 | 50 |
| Ac53 | 20 | 58 | 58 | 99 | 98 | 20 | 46 | 0 | 84 |
| | 15 | 25 | 25 | 41 | 97 | 6 | 32 | 0 | 84 |
| | 10 | 3 | 3 | 20 | 74 | 8 | 22 | 0 | 79 |
| | 5 | 0 | 0 | 7 | 26 | 4 | 0 | 0 | 69 |
| Ac56 | 20 | 55 | 55 | 84 | 96 | 17 | 35 | 0 | 78 |
| | 15 | 23 | 23 | 48 | 96 | 15 | 16 | 0 | 72 |
| | 10 | 0 | 0 | 6 | 79 | 10 | 24 | 0 | 61 |
| | 5 | 0 | 0 | 5 | 44 | 0 | 5 | 0 | 24 |
| Ac59 | 20 | 90 | 90 | 83 | 20 | 5 | 7 | 0 | 0 |
| | 15 | 92 | 92 | 5 | 14 | 0 | 15 | 0 | 0 |
| | 10 | 82 | 82 | 10 | 16 | 0 | 0 | 0 | 0 |
| | 5 | 13 | 13 | 0 | 13 | 0 | 0 | 1 | 10 |
| Ac66 | 20 | 31 | 45 | 99 | 100 | 27 | 0 | 0 | |
| | 17.5 | 15 | 7 | 3 | 98 | 20 | 0 | 0 | |
| | 15 | 5 | 2 | 0 | 83 | 14 | 0 | 0 | |
| | 12.5 | 11 | 3 | 0 | 39 | 14 | 0 | 0 | |
| Ac67 | 20 | 23 | 28 | 100 | 100 | 31 | 0 | 0 | |
| | 17.5 | 22 | 14 | 37 | 100 | 31 | 0 | 0 | |
| | 15 | 3 | 0 | 0 | 91 | 27 | 0 | 0 | |
| | 12.5 | 12 | 0 | 0 | 75 | 23 | 0 | 0 | |
| Ac68 | 20 | 15 | 0 | 84 | 31 | 0 | 0 | 0 | |
| | 17.5 | 11 | 0 | 90 | 44 | 4 | 0 | 0 | |
| | 15 | 0 | 0 | 94 | 28 | 10 | 0 | 0 | |
| | 12.5 | 0 | 3 | 98 | 43 | 11 | 0 | 0 | |
| Ac88 | 20 | 71 | 71 | 85 | 86 | 12 | 13 | 0 | 50 |
| | 15 | 34 | 34 | 41 | 93 | 7 | 0 | 0 | 58 |
| | 10 | 15 | 15 | 0 | 73 | 4 | 0 | 0 | 47 |
| | 5 | 0 | 0 | 0 | 0 | 0 | 0 | 0 | 0 |
| Ac96 | 20 | 44 | 53 | 97 | 100 | 37 | 0 | 0 | |
| | 17.5 | 4 | 0 | 0 | 99 | 29 | 0 | 0 | |
| | 15 | 0 | 4 | 0 | 80 | 14 | 6 | 0 | |
| | 12.5 | 9 | 0 | 0 | 69 | 14 | 0 | 1 | |
| Ac97 | 20 | 18 | 12 | 11 | 100 | 20 | 0 | 0 | |
| | 17.5 | 11 | 3 | 0 | 96 | 18 | 2 | 1 | |
| | 15 | 0 | 0 | 0 | 64 | 14 | 0 | 1 | |
| | 12.5 | 1 | 0 | 0 | 32 | 9 | 0 | 0 | |
| Ac98 | 20 | 28 | 4 | 30 | 100 | 22 | 0 | 0 | |
| | 17.5 | 15 | 0 | 3 | 94 | 16 | 0 | 8 | |
| | 15 | 0 | 0 | 0 | 70 | 13 | 0 | 11 | |
| | 12.5 | 0 | 0 | 0 | 31 | 10 | 0 | 5 | |
| Ac99 | 20 | 27 | 32 | 92 | 100 | 27 | 4 | 0 | |
| | 17.5 | 14 | 1 | 7 | 99 | 17 | 0 | 0 | |
| | 15 | 20 | 1 | 1 | 80 | 23 | 5 | 21 | |
| | 12.5 | 9 | 0 | 0 | 60 | 16 | 0 | 8 | |
| Ac100 | 20 | 39 | 37 | 78 | 100 | 34 | 6 | 0 | |
| | 17.5 | 23 | 9 | 0 | 98 | 28 | 0 | 0 | |
| | 15 | 11 | 0 | 0 | 51 | 25 | 4 | 7 | |
| | 12.5 | 8 | 0 | 0 | 70 | 20 | 0 | 7 | |
| Ac101 | 20 | 23 | 42 | 99 | 100 | 25 | 29 | 0 | |
| | 17.5 | 11 | 7 | 100 | 100 | 22 | 0 | 1 | |
| | 15 | 0 | 0 | 9 | 91 | 25 | 0 | 9 | |
| | 12.5 | 0 | 0 | 0 | 82 | 16 | 0 | 0 | |
| Ac102 | 20 | 14 | 14 | 96 | 100 | 21 | 0 | 4 | |
| | 17.5 | 14 | 0 | 99 | 98 | 0 | 0 | 0 | |
| | 15 | 0 | 0 | 99 | 77 | 24 | 0 | 0 | |
| | 12.5 | 11 | 0 | 75 | 77 | 17 | 0 | 0 | |
| Ac103 | 20 | 8 | 1 | 28 | 99 | 16 | 0 | 0 | |
| | 17.5 | 0 | 0 | 0 | 97 | 11 | 0 | 0 | |
| | 15 | 0 | 0 | 0 | 80 | 8 | 0 | 0 | |
| | 12.5 | 0 | 0 | 0 | 61 | 14 | 18 | 0 | |
| Ac116 | 20 | 13 | 0 | 100 | 18 | 9 | 0 | 0 | |
| | 17.5 | 23 | 7 | 100 | 31 | 24 | 4 | 9 | |
| | 15 | 17 | 7 | 100 | 22 | 11 | 0 | 0 | |
| | 12.5 | 7 | 8 | 100 | 25 | 9 | 0 | 5 | |
| Ac138 | 20 | 22 | 0 | 86 | 1 | 0 | 0 | 0 | |
| | 17.5 | 16 | 0 | 40 | 1 | 11 | 0 | 0 | |
| | 15 | 17 | 11 | 46 | 9 | 3 | 0 | 0 | |
| | 12.5 | 20 | 18 | 18 | 19 | 10 | 0 | 0 | |
| Ac173 | 20 | 0 | 0 | 100 | 0 | 4 | 0 | 5 | |
| | 17.5 | 0 | 0 | 100 | 1 | 7 | 0 | 7 | |
| | 15 | 10 | 4 | 100 | 11 | 7 | 0 | 7 | |
| | 12.5 | 15 | 13 | 61 | 3 | 8 | 0 | 0 | |
| Ac175 | 20 | 13 | 0 | 0 | 100 | 23 | 4 | 0 | |
| | 15 | 0 | 0 | 0 | 91 | 11 | 0 | 7 | |
| | 10 | 0 | 0 | 0 | 65 | 5 | 0 | 0 | |
| | 5 | 18 | 6 | 0 | 27 | 9 | 3 | 0 | |
| Ac177 | 20 | 99 | 99 | 100 | 100 | 83 | 0 | 15 | |
| | 15 | 100 | 99 | 100 | 91 | 88 | 0 | 0 | |
| | 10 | 100 | 99 | 98 | 57 | 92 | 0 | 0 | |
| | 5 | 99 | 99 | 100 | 32 | 99 | 0 | 0 | |
| Ac178 | 20 | 21 | 0 | 100 | 16 | 0 | 0 | 13 | |
| | 15 | 13 | 0 | 82 | 7 | 5 | 0 | 30 | |
| | 10 | 18 | 8 | 23 | 5 | 2 | 0 | 0 | |
| | 5 | 7 | 11 | 12 | 0 | 0 | 0 | 0 | |

Ac175, Control peptide SRCRP2;
Ac177, Control peptide tachyplesin-1;
Ac178, Control peptide Magainin-2

TABLE 7

| Strongly inhibitory | | | | |
|---|---|---|---|---|
| against A. baumannii | | | Weakly inhibitory | |
| | MIC (μM) for | MIC (μM) | against A. baumannii | |
| | non- amidated | for amidated | | MIC |
| Peptide | Peptide | peptide | Peptide | (μM) |
| Ac5 | 20-40 | 5-10 | Ac319 | 50 |
| Ac5r¹ | 15 | 5 | Ac329 | 30 |
| Ac8 | >40 | 20 | Ac338 | 30 |
| Ac13 | 2.5 | 2.5 | Ac340 | 40 |
| Ac13r¹ | 1.25-2.5 | 1.25-2.5 | Ac364 | 40 |
| Ac14 | 5 | 2.5 | Ac379 | 30 |
| Ac16 | 10-15 | | Ac380 | 40 |
| Ac17 | >40 | 15 | Ac390 | 40 |
| Ac35 | 20 | 20 | Ac434 | 40 |
| Ac38 | 40 | 15-20 | Ac437 | 40 |
| Ac39 | 10-15 | 10-15 | Ac451 | 40 |
| Ac40 | 10-15 | 10-15 | Ac469 | 50 |
| Ac44 | >40 | 20 | Ac496 | 40 |
| Ac59 | 20 | 20 | | |
| Ac193 | >40 | 20 | | |
| Ac195 | 20 | 10 | | |
| Ac228 | 20-25 | | | |
| Ac259 | 2.5 | 3.8-4.0 | | |
| Ac322 | 5 | | | |
| Ac328 | 12.5 | | | |
| Ac330 | 20 | | | |
| Ac339 | 20 | | | |
| Ac370 | 20 | | | |
| Ac378 | 12.5 | | | |
| Ac389 | 12.5 | | | |
| Ac431 | 15 | | | |
| Ac436 | 10 | | | |
| Ac476 | 5 | | | |
| Ac472 | 15 | | | |
| Ac474 | 25 | | | |
| Ac475 | 15 | | | |
| Ac323 | 20 | | | |
| Ac327 | 20 | | | |

¹r denotes retroinverso peptide

TABLE 8

| Strongly inhibitory | | | Weakly inhibitory | | |
|---|---|---|---|---|---|
| against A. baumannii | | | against A. baumannii | | |
| Peptide | MIC (μM) | MBC (μM) | Peptide | MIC (μM) | MBC (μM) |
| Ac228 | 25 | bacteriostat | Ac319 | 50 | bacteriostat |
| Ac259 | 4 | 6 | Ac329 | 30 | 39 |
| Ac322 | 5 | 5 | Ac338 | 30 | 30 |
| Ac323 | 20 | 20 | Ac340 | 40 | 40 |
| Ac327 | 20 | 20 | Ac364 | 40 | 40 |
| Ac328 | 12.5 | 16.25 | Ac379 | 30 | 39 |
| Ac330 | 20 | 40 | Ac380 | 40 | n.d. |
| Ac339 | 20 | 40 | Ac390 | 40 | 40 |
| Ac370 | 20 | 20 | Ac434 | 40 | 40 |
| Ac378 | 12.5 | 12.5 | Ac437 | 40 | n.d. |
| Ac389 | 12.5 | 20 | Ac451 | 40 | n.d. |
| Ac431 | 15 | 15 | Ac469 | 50 | bacteriostat |
| Ac436 | 10 | 10 | Ac496 | 40 | 40 |
| Ac476 | 5 | 5 | | | |
| Ac472 | 15 | bacteriostat | | | |
| Ac474 | 25 | 25 | | | |
| Ac475 | 15 | 15 | | | |

### EXAMPLE 4

### Antimicrobial Peptides have Low Toxicities to Mammalian Cells

This example demonstrates the cytotoxicities of antibacterial peptides as determined by hemolysis of red blood cells (RBC), and fluorescence of Jurkat cells using Celltiter-Blue dye, in the presence and absence of peptide.

Methods

a) Hemolysis of RBC

Fresh human RBC obtained previously from volunteers were washed in an isotonic buffer until the supernatant showed no coloration. Varying amounts of peptide were incubated with the washed RBC for one hour at room temperature. Intact cells were pelleted by gentle centrifugation, and hemolysis of each sample was determined by absorption measurement at 450 nm. A positive control sample consisted of RBC lysed by the addition of 0.1% Triton, for which hemolysis was also determined by absorption measurement at 450 nm. Percentages of lysed RBC incubated with peptide were then calculated by the ratio of absorbances at 450 nm for the sample relative to the absorbance at 450 nm of the positive control sample. The MHC₁₀ value was calculated as the minimum peptide concentration to induce 10% cell lysis.

b) Cytotoxicity of Peptides Against Jurkat Cells

The cytotoxicities of selected peptides against human T-lymphocytes, in particular Jurkat cells, was determined by growing 100 uL Jurkat cells in RPMI (approx. 5×10⁵ cells/ml density) overnight at 37° C. under 5% CO₂ atmosphere, in microtiter plate wells, in the presence and absence of peptides. Control reactions either lacked peptide (negative cytotoxicity control), or contained a buffer to completely lyse Jurkat cells (positive cytotoxicity control). The viabilities of cells were then determined by addition of 20 uL CellTiter Blue dye (Promega) to each well, and incubation of cells for a further 3 hrs at 37° C. Fluorescence values were measured for 1 sec at 540 nm/615 nm, and expressed relative to fluorescence values for negative and positive cytotoxicity control samples. Comparison with a fully lysed control allowed the calculation of a percentage killing for each peptide concentration. The MCC₁₀ value was calculated as the minimum peptide concentration required to induce 10% cytotoxicity.

Results

Data shown in Table 9 demonstrate haemolytic activities of peptides in Jurkat cells. Peptides having less than 1% haemolytic activity at 100 μM concentration were deemed non-hemolytic. As shown in Table 9, peptides designated Ac13, Ac228 and Ac259 had reproducibly higher haemolytic activities under these conditions. In contrast peptides designated Ac5, Ac8, Ac14, Ac53, Ac59, Ac67, Ac193, Ac195, and Ac319 were deemed non-hemolytic under these conditions. Data for these peptides compared favourably to tachyplesin-1, which induced 5% hemolysis of RBC at 20 μM concentration.

Date presented in Table 10 demonstrate cytotoxicities for the peptides in Jurkat cells. Peptides having less than 1% cytotoxicity at 100 μM concentration were deemed non-cytotoxic. As shown in Table 10, peptides designated Ac5, Ac14, Ac17, Ac38 and Ac195 were deemed non-cytotoxic to Jurkat cells under these conditions. Peptides Ac5, Ac14 and Ac195 were also non-hemolytic to RBC. Data for these peptides compared favourably to tachyplesin-1, which killed Jurkat 10% cells at 20 μM concentration.

TABLE 9

| | | Percent hemolysis at each peptide | | | | | | | | |
|---|---|---|---|---|---|---|---|---|---|---|
| | | concentration (μM) | | | | | | | | |
| Peptide | | 100 | 80 | 60 | 40 | 20 | 10 | 5 | 2.5 | MHC₁₀ |
| 1 | Ac5 | 3.3 | 2.6 | 1 | 0.9 | 1.3 | 1.0 | 0.7 | 0.6 | >100 |
| 2 | Ac8 | 0.3 | 0.3 | 0.2 | 0.3 | 0 | 0.3 | 0.3 | 0.8 | Non-hemolytic |
| 3 | Ac13 | 27.6 | 25.3 | 29.3 | 26.3 | 26.8 | 7.9 | 2.1 | 1.6 | ~12-20 |
| 4 | Ac17 | 4.5 | 2.4 | 0.9 | 0.4 | 0.2 | 0.1 | 0.2 | 0.7 | >100 |
| 5 | Ac35 | n.d. | n.d. | n.d. | n.d. | 4.4 | 5.9 | 0.5 | 0.5 | >100 |
| 6 | Ac38 | 27.9 | 26.9 | 19.5 | 11.9 | 4.8 | 3.8 | 0.8 | 0.4 | >100 |
| 7 | Ac44 | 0.3 | 0.3 | 0.3 | 0.1 | 0.9 | 0.6 | n.d | n.d | Non-hemolytic |
| 8 | Ac53 | 0.6 | 0.5 | 0.5 | 0.4 | 0.4 | 0.8 | n.d | n.d | Non-hemolytic |
| 9 | Ac59 | 0.3 | 0.1 | 0.1 | 0.2 | 0.4 | 0.4 | 0.6 | 0.8 | Non-hemolytic |
| 10 | Ac67 | 0.2 | 0.1 | 0.1 | 0 | 0.5 | 0.7 | n.d | n.d | Non-hemolytic |
| 11 | Ac193 | 0.1 | 0.1 | n.d. | n.d. | 0 | 0 | 0.2 | 0.6 | Non-hemolytic |
| 12 | Ac195 | 0.4 | 0.2 | 0.1 | 0.1 | 0.5 | 0.1 | 0.1 | 0.6 | Non-hemolytic |
| 13 | Ac201 | | | | | | | | | 70 |
| 14 | Ac228 | 97.1 | 80.4 | 40.1 | 22.9 | 9.9 | 11.0 | 2.6 | 1.1 | ~10 |
| 15 | Ac259 | 87.9 | 66 | 47.4 | 24.9 | 2.2 | 1.2 | 0.8 | 0.6 | 25-35 |
| 16 | Ac317 | | | | | | | | | 30 |
| 17 | Ac319 | | | | | | | | | >100 |
| 18 | Ac322 | | | | | | | | | 25 |
| 19 | Ac323 | | | | | | | | | 30 |
| 20 | Ac327 | | | | | | | | | 30 |
| 21 | Ac328 | | | | | | | | | 20 |
| 22 | Ac378 | | | | | | | | | 50 |
| 23 | Ac389 | | | | | | | | | 50 |
| 24 | Ac431 | | | | | | | | | 25 |
| 25 | Ac436 | | | | | | | | | 75 |
| 26 | Ac474 | | | | | | | | | 5 |
| 27 | Ac475 | | | | | | | | | 10 |
| 28 | Ac476 | | | | | | | | | 50 |

Analog

| | | | | | | | | | | |
|---|---|---|---|---|---|---|---|---|---|---|
| 29 | Ac14 | 4.7 | 5.6 | 1 | 2.1 | 0.4 | n.d. | n.d. | n.d. | >100 |
| 30 | Ac39 | 11 | 9 | 5.2 | 3.3 | 0.8 | n.d. | n.d. | n.d. | ~90 |
| 31 | Ac13r¹ | | | | | | | | | ~15 |

¹, r denotes retroinverso peptide analog

TABLE 10

| | | Percent cytotoxicity at each peptide | | | |
|---|---|---|---|---|---|
| | | concentration (μM) | | | |
| Peptide | | 50 | 25 | 12.5 | MCC₁₀ |
| 1 | Ac5 | 0 | 0 | 0 | Non-cytotoxic |
| 2 | Ac13 | 43 | 0 | 0 | 30 |
| 3 | Ac17 | 0 | 0 | 0 | Non-cytotoxic |
| 4 | Ac35 | 32 | 0 | 0 | 32 |
| 5 | Ac38 | 0 | 0 | 0 | Non-cytotoxic |
| 6 | Ac195 | 1 | 8 | 4 | Non-cytotoxic |
| 7 | Ac201 | 23 | 0 | 0 | 42 |
| 8 | Ac228 | 29 | 5 | 0 | 42 |
| 9 | Ac259 | 86 | 28 | 4 | 20 |
| 10 | Ac317 | 0 | 12 | 2 | 23 |
| 11 | Ac319 | 36 | 6 | 2 | 30 |
| 12 | Ac322 | 59 | 0 | 0 | 30 |
| 13 | Ac323 | 49 | 31 | 9 | 13 |
| 14 | Ac327 | 1 | 31 | 20 | 8 |
| 15 | Ac328 | 32 | 17 | 1 | 20 |
| 16 | Ac378 | 35 | 0 | 0 | 30 |
| 17 | Ac389 | 95 | 34 | 0 | 15 |
| 18 | Ac431 | 29 | 15 | 4 | 20 |
| 19 | Ac436 | 32 | 10 | 6 | 25 |
| 20 | Ac474 | 81 | 20 | 13 | 10 |
| 21 | Ac475 | 60 | 4 | 4 | 35 |
| 22 | Ac476 | 44 | 18 | 12 | 10 |
| 23 | Ac14 | 0 | 0 | 3 | Non-cytotoxic |
| 24 | Ac13r¹ | 39 | 3 | 0 | 30 |

¹r denotes retroinverso peptide analog

### EXAMPLE 5

### Effect of Peptides in Reducing A. baumannii Infection in Lungs

This example demonstrates the efficacy of peptides that are cytotoxic against A. baumannii in reducing cell counts in infected lung tissue in an accepted animal model of A. baumannii infection.

Methods

Groups of C57B1/6 mice that have been ear-tagged for identification are used. Freshly-prepared inocula of A. baumannii are prepared from cultures of A. baumannii passaged four times through mice, and cultures are grown with agitation at 37° C. in Nutrient broth to an OD₆₀₀ value of approximately 0.3 units. Cells are collected by centrifugation at room temperature for 20 mins (5,500 rpm using a GA-10 rotor) and resuspended in 10 ml sterile PBS at room temperature. This wash is repeated once. The cell pellet is then resuspended in 0.5 ml PBS to an approximate concentration of 2.5×10⁹ c.f.u./ml. Animals are inoculated intranasally 40 μl inoculum (approximately 1×10⁸ c.f.u.) using a sterile 200 μl capacity pipette tip having approximately 3 mm removed from the end. The precise time of inoculation is recorded for each animal. Peptides are administered intravenously e.g., after 3 hr, in 200 μl aliquots of a 200 μM peptide solution in PBS.

In the specific example herein, mice were injected with 200 μL of 200 μM retroinverso peptide analog of peptide Ac13 (end conc. 20 μM) or 200 μL of 250 μM peptide Ac259 (end conc. 25 μM) or 200 μL PBS. Similar trials are performed using any one or more of the peptides presented in Tables 1-10 hereof or presented in the Sequence Listing.

At 5.5 hr post-inoculation (taking into account staggered inoculation times), both lung lobes are removed from the animals, homogenised in 0.9 ml PBS and plated on MHB plates at 1×10⁻³ dilution, 1×10⁻⁴ dilution and 1×10⁻⁵ dilution in PBS. Following incubation periods sufficient to grow colonies, cells are counted.

Results

Data presented in Table 11 indicate a strong trend for the retroinverso analog of peptide Ac13 to reduce A. baumannii infection in the lung, and a less-pronounced trend for peptide Ac259 to reduce A. baumannii infection in the lung.

TABLE 11

| | Cell counts in lungs post-treatment | | |
|---|---|---|---|
| | (% inhibition relative to PBS control) | | |
| Treatment | Mouse 1 | Mouse 2 | Mouse 3 |
| Retroinverse | 1 × 10⁴ (99.5%) | 2.6 × 10⁵ (87.8%) | 9.0 × 10⁴ (95.8%) |
| Ac13 | | | |
| Ac259 | 7.9 × 10⁵ (63.0%) | 1.8 × 10⁵ (91.6%) | 1.5 × 10⁵ (93.0%) |
| PBS control | 6.1 × 10⁵ | 4.1 × 10⁶ | 1.7 × 10⁶ |

1. An antimicrobial peptide or analog or derivative thereof having low or non-detectable cytotoxicity against mammalian red blood cells or T-lymphocytes and having cytotoxicity against at least one gram-negative bacterium of the genus Acenitobacter or that is or capable of reducing or preventing growth of at least one bacterium of the genus Acenitobacter, wherein said bacterium comprises a laboratory isolate or clinical isolate of A. baumannii.

2-3. (canceled)

4. The antimicrobial peptide or analog or derivative thereof according to claim 3, wherein said peptide, analog or derivative is cytotoxic to less than 10% of a culture of red blood cells or T-lymphocytes when present at a concentration of 100 μM or greater.

5. The antimicrobial peptide or analog or derivative thereof according to claim 1, wherein said peptide, analog or derivative has weak cytotoxicity or is weakly cytostatic against Escherichia coli.

6. The antimicrobial peptide or analog or derivative thereof according to claim 1, wherein said peptide, analog or derivative has weak cytotoxicity or is weakly cytostatic against one or more of Escherichia coli strain BL21, S. typhimurium strain AroA, P. aeruginosa or P. pneumotropica.

7. The antimicrobial peptide or analog or derivative thereof according to claim 1, wherein said peptide, analog or derivative has a minimum inhibitory concentration toward A. baumannii of 25 μM or less.

8. The antimicrobial peptide or analog or derivative thereof according to claim 1, wherein said peptide, analog or derivative is cytotoxic against a plurality of Acenitobacter spp. or reduces or prevents growth of a plurality of Acenitobacter spp., subject to said plurality at least comprising a laboratory isolate or clinical isolate of A. baumannii.

9. (canceled)

10. The antimicrobial peptide or analog or derivative thereof according to claim 8, wherein the plurality of Acenitobacter spp. comprise one or more laboratory and/or clinical isolates of A. baumannii and one or both of ATCC Accession No. 17903 and ATCC Accession No. 19004.

11. The antimicrobial peptide or analog or derivative thereof according to claim 1, having cytotoxic activity or bacteriostatic activity against a plurality of Acenitobacter spp. and against S. aureus, subject to said plurality at least comprising a laboratory isolate or clinical isolate of A. baumannii.

12. A fusion protein comprising one or more antimicrobial peptides or analogs or derivatives thereof according to claim 1.

13. A formulation comprising an effective amount of one or more antimicrobial peptides, analogs or derivatives according to claim 1 and/or one or more fusion proteins comprising said one or more antimicrobial peptides, analogs or derivatives and a suitable carrier or excipient.

14. A solid surface coated with or having adsorbed thereto one or more antimicrobial peptides, analogs or derivatives according to claim 1 and/or one or more fusion proteins comprising said one or more antimicrobial peptides, analogs or derivatives.

15-16. (canceled)

17. A method for killing at least one gram-negative bacterium of the genus Acenitobacter or for reducing or preventing growth of at least one bacterium of the genus Acenitobacter, said method comprising contacting the gram-negative bacterium or a surface or composition of matter suspected of being contaminated with the gram-negative bacterium with one or more antimicrobial peptides, analogs or derivatives according to claim 1 and/or one or more fusion proteins comprising said one or more antimicrobial peptides, analogs or derivatives for a time and under conditions sufficient to kill the gram-negative bacterium or to reduce or prevent growth thereof.

18. (canceled)

19. A method of therapeutic or prophylactic treatment of a subject comprising administering one or more antimicrobial peptides, analogs or derivatives according to claim 1 and/or one or more fusion proteins comprising said one or more antimicrobial peptides, analogs or derivatives to a subject in need thereof.

20. A method of therapeutic or prophylactic treatment of a subject for bacterial infection, said method comprising:
(i) determining a subject suffering from a bacterial infection or at risk of developing a bacterial infection;
(ii) obtaining one or more antimicrobial peptides, analogs or derivatives according to claim 1 and/or one or more fusion proteins comprising said one or more antimicrobial peptides, analogs or derivatives and/or a formulation comprising said peptides, analogs, derivatives or fusion proteins; and
(iii) administering said peptide or analog or derivative or fusion protein or formulation to said subject for a time and under conditions sufficient to reduce or prevent bacterial infection.

21. A method for the prophylactic or therapeutic treatment of a bacterial infection, said method comprising:
(i) identifying a subject suffering from an infection or suspected of suffering from a bacterial infection or at risk of developing a bacterial infection; and
(ii) recommending administration of one or more antimicrobial peptides, analogs or derivatives according to claim 1 and/or one or more fusion proteins comprising said one or more antimicrobial peptides, analogs or derivatives and/or a formulation comprising said peptides, analogs, derivatives or fusion proteins sufficient to reduce or prevent bacterial infection.

22. A method of prophylactic or therapeutic treatment of a bacterial infection in a subject, said method comprising administering or recommending administration of one or more antimicrobial peptides, analogs or derivatives according to claim 1 and/or one or more fusion proteins comprising said one or more antimicrobial peptides, analogs or derivatives and/or a formulation comprising said peptides, analogs, derivatives or fusion proteins sufficient to reduce or prevent bacterial infection in the subject.

23. A method of peptide synthesis comprising obtaining a sequence of an antimicrobial peptide or analog or derivative according to claim 1 or a fusion protein fusion protein comprising said antimicrobial peptide or analog or derivative, and synthesizing the peptide, analog, derivative or fusion protein.

24. (canceled)

25. A method of peptide production comprising obtaining a sequence of an antimicrobial peptide or analog or derivative according to claim 1 or a fusion protein fusion protein comprising said antimicrobial peptide or analog or derivative, and performing a process of mutation or affinity maturation of the peptide, analog or derivative or otherwise altering the sequence of the peptide, analog or derivative to thereby produce a peptide having enhanced antimicrobial activity to toward one or more bacteria.

26. The method of claim 25, additionally comprising isolating the peptide having enhanced antimicrobial activity toward one or more bacteria.

27-28. (canceled)

29. A peptide or analog or derivative isolated by the method of claim 26.

30. A method for isolating a compound that reduces or prevents growth of a microorganism and/or kills a microorganism, said method comprising:
(i) determining the structure of an antimicrobial peptide or analog or derivative according to claim 1 or a fusion protein fusion protein comprising said antimicrobial peptide or analog or derivative;
(ii) identifying, producing or obtaining one or more compounds that have a similar structure to the peptide, analog, derivative or fusion protein at (i) or that are predicted to have a similar structure to the peptide, analog, derivative or fusion protein at (i);
(iii) determining the ability of the one or more compounds at (ii) to reduce or prevent growth of a microorganism and/or kill a microorganism; and
(iv) isolating a compound that reduces or prevents growth of a microorganism and/or kills a microorganism.

31. A compound isolated by the method of claim 30.

32. (canceled)

33. A method for prolonging the storage life of a perishable product, said method comprising:
(i) contacting a perishable product with one or more antimicrobial peptides, analogs or derivatives according to claim 1 and/or one or more fusion proteins comprising said one or more antimicrobial peptides, analogs or derivatives and/or a formulation comprising said peptides, analogs, derivatives or fusion proteins for a time and under conditions sufficient to reduce or prevent growth of a microorganism and/or to kill a microorganism; and
(ii) storing the perishable product for a time that is longer than the time the product would have been stored in the absence of contact with the peptide, analog, derivative, fusion protein or formulation.
